SEQUENCE LISTING (277 sequences)

SEQ ID NO: 1 - protein, 20 aa, Homo sapiens
AVIRYSVAIILFTILPFFLL

SEQ ID NO: 2 - protein, 23 aa, Homo sapiens
YIWAPLAGTCGVLLLSLVITLYC

SEQ ID NO: 3 - protein, 27 aa, Homo sapiens
FWVLVVVGGVLACYSLLVTVAFIIFWV

SEQ ID NO: 4 - protein, 46 aa, Homo sapiens
TTTPAPRPPTPAPTIASQPLSLRPEACRPAAGGAVHTRGLDFACDI

SEQ ID NO: 5 - protein, 39 aa, Homo sapiens
IEVMYPPPYLDNEKSNGTIIHVKGKHLCPSPLFPGPSKP

SEQ ID NO: 6 - protein, 115 aa, Homo sapiens
HRWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 7 - protein, 115 aa, synthetic construct
HRWCSKKKDAAVMNQEPAGHRTVNREDSDEQDPQEVAFAQLDHCIFTQRKITGPSQRSKRPSTDTSVCIELPNAEPRALSPAHEHHSQALMGSSRETTALSQTQLASSNVPAAGI

SEQ ID NO: 8 - protein, 29 aa, Homo sapiens
HRWCSKKKDAAVMNQEPAGHRTVNREDSD

SEQ ID NO: 9 - protein, 10 aa, Homo sapiens
HRWCSKKKDA

SEQ ID NO: 10 - protein, 49 aa, synthetic construct
HRWCSKKKDAAVMNQEPAGHRTVNREDSDEQDPQEVAFAQLDHCIFTQR

SEQ ID NO: 11 - protein, 58 aa, synthetic construct
HRWCSKKKDAAVMNQEPAGHRTVNREDSDEQDPQEVAFAQLDHCIFTQRKITGPSQRS

SEQ ID NO: 12 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 41 aa, Homo sapiens
RSKRSRLLHSDYMNMTPRRPGPTRKHYQPYAPPRDFAAYRS

SEQ ID NO: 14 - protein, 42 aa, Homo sapiens
KRGRKKLLYIFKQPFMRPVQTTQEEDGCSCRFPEEEEGGCEL

SEQ ID NO: 15 - protein, 24 aa, Homo sapiens
LCARPRRSPAQEDGKVYINMPGRG

SEQ ID NO: 16 - protein, 52 aa, synthetic construct
YFLGRLVPRGRGAAEAATRKQRITETESPYQELQGQRSDVYSDLNTQRPYYK

SEQ ID NO: 17 - protein, 135 aa, synthetic construct
AVIRYSVAIILFTILPFFLLHRWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 18 - protein, 247 aa, synthetic construct
AVIRYSVAIILFTILPFFLLHRWCSKKKDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 49 aa, synthetic construct
AVIRYSVAIILFTILPFFLLHRWCSKKKDAAVMNQEPAGHRTVNREDSD

SEQ ID NO: 20 - protein, 161 aa, synthetic construct
AVIRYSVAIILFTILPFFLLHRWCSKKKDAAVMNQEPAGHRTVNREDSDRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 135 aa, synthetic construct
AVIRYSVAIILFTILPFFLLHRWCSKKKDAAVMNQEPAGHRTVNREDSDEQDPQEVAFAQLDHCIFTQRKITGPSQRSKRPSTDTSVCIELPNAEPRALSPAHEHHSQALMGSSRETTALSQTQLASSNVPAAGI

SEQ ID NO: 22 - protein, 247 aa, synthetic construct
AVIRYSVAIILFTILPFFLLHRWCSKKKDAAVMNQEPAGHRTVNREDSDEQDPQEVAFAQLDHCIFTQRKITGPSQRSKRPSTDTSVCIELPNAEPRALSPAHEHHSQALMGSSRETTALSQTQLASSNVPAAGIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 69 aa, synthetic construct
AVIRYSVAIILFTILPFFLLHRWCSKKKDAAVMNQEPAGHRTVNREDSDEQDPQEVAFAQLDHCIFTQR

SEQ ID NO: 24 - protein, 181 aa, synthetic construct
AVIRYSVAIILFTILPFFLLHRWCSKKKDAAVMNQEPAGHRTVNREDSDEQDPQEVAFAQLDHCIFTQRRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 78 aa, synthetic construct
AVIRYSVAIILFTILPFFLLHRWCSKKKDAAVMNQEPAGHRTVNREDSDEQDPQEVAFAQLDHCIFTQRKITGPSQRS

SEQ ID NO: 26 - protein, 190 aa, synthetic construct
AVIRYSVAIILFTILPFFLLHRWCSKKKDAAVMNQEPAGHRTVNREDSDEQDPQEVAFAQLDHCIFTQRKITGPSQRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 135 aa, synthetic construct
YIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 138 aa, synthetic construct
YIWAPLAGTCGVLLLSLVITLYCHRWCSKKKDAAVMNQEPAGHRTVNREDSDEQDPQEVAFAQLDHCIFTQRKITGPSQRSKRPSTDTSVCIELPNAEPRALSPAHEHHSQALMGSSRETTALSQTQLASSNVPAAGI

SEQ ID NO: 29 - protein, 181 aa, synthetic construct
FWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 219 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 177 aa, synthetic construct
YIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 69 aa, synthetic construct
AVIRYSVAIILFTILPFFLLHRWCSKKKDAAVMNQEPAGHRTVNREDSDEQDPQEVTYAQLDHCIFTQR

SEQ ID NO: 33 - protein, 111 aa, synthetic construct
AVIRYSVAIILFTILPFFLLHRWCSKKKDAAVMNQEPAGHRTVNREDSDEQDPQEVTYAQLDHCIFTQRKRGRKKLLYIFKQPFMRPVQTTQEEDGCSCRFPEEEEGGCEL

SEQ ID NO: 34 - protein, 93 aa, synthetic construct
AVIRYSVAIILFTILPFFLLHRWCSKKKDAAVMNQEPAGHRTVNREDSDEQDPQEVTYAQLDHCIFTQRLCARPRRSPAQEDGKVYINMPGRG

SEQ ID NO: 35 - protein, 241 aa, synthetic construct
EVQLVETGGGLVQPGGSLRLSCAVSGFALSNHGMSWVRRAPGKGLEWCCVSGIVYSGSTYYAASVKGRFTISRDNSRNTLYLQMNSLRPEDTAIYYCSAHGGESDVWGQGTTVTVSSASGGGGSGGRASGGGGSEIVMTQSPATLSVSPGERATLSCRASQSVSSKLAWYQQKPGQAPRLLMYGASIRATGIPDRFSGSGSGTEFTLTISSLEPEDFAVYYCQQYGSSSWTFGQGTKVEIK

SEQ ID NO: 36 - protein, 239 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARESGDGMDVWGQGTTVTVSSASGGGGSGGRASGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYTLAFGQGTKVDIK

SEQ ID NO: 37 - protein, 239 aa, synthetic construct
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 38 - protein, 239 aa, synthetic construct
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 39 - protein, 240 aa, synthetic construct
EVQLVETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSETTLTQSPATLSVSPGERATLSCRASQSVGSNLAWYQQKPGQGPRLLIYGASTRATGIPARFSGSGSGTEFTLTISSLQPEDFAVYYCQQYNDWLPVTFGQGTKVEIK

SEQ ID NO: 40 - protein, 241 aa, synthetic construct
EVQLVETGGGVVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSVGSTNLAWYQQKPGQAPRLLIYDASNRATGIPDRFSGGGSGTDFTLTISRLEPEDFAVYYCQQYGSSPPWTFGQGTKVEIK

SEQ ID NO: 41 - protein, 241 aa, synthetic construct
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 42 - protein, 244 aa, synthetic construct
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCSVHSFLAYWGQGTLVTVSSASGGGGSGGRASGGGGSDIVMTQTPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKVEIK

SEQ ID NO: 43 - protein, 246 aa, synthetic construct
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 44 - protein, 246 aa, synthetic construct
QVQLQESGGGLVQPGGSLRLSCAASGFTFSSDAMTWVRQAPGKGLEWVSVISGSGGTTYYADSVKGRFTISRDNSKNTLYLQMNSLRAEDTAVYYCAKLDSSGYYYARGPRYWGQGTLVTVSSASGGGGSGGRASGGGGSDIQLTQSPSSLSASVGDRVTITCRASQSISSYLNWYQQKPGKAPKLLIYGASTLASGVPARFSGSGSGTHFTLTINSLQSEDSATYYCQQSYKRASFGQGTKVEIK

SEQ ID NO: 45 - protein, 246 aa, synthetic construct
QVQLVQSGGGLVKPGGSLRLSCAASGFTFSDYYMSWIRQAPGKGLEWVSYISSSGNTIYYADSVKGRFTISRDNAKNSLYLQMNSLRAEDTAVYYCARSTMVREDYWGQGTLVTVSSASGGGGSGGRASGGGGSDIVLTQSPLSLPVTLGQPASISCKSSESLVHNSGKTYLNWFHQRPGQSPRRLIYEVSNRDSGVPDRFTGSGSGTDFTLKISRVEAEDVGVYYCMQGTHWPGTFGQGTKLEIK

SEQ ID NO: 46 - protein, 247 aa, synthetic construct
QVQLVQSGAEVKKPGASVKVSCKASGYTFSNYGITWVRQAPGQGLEWMGWISAYNGNTNYAQKFQGRVTMTRNTSISTAYMELSSLRSEDTAVYYCARGPYYYYMDVWGKGTMVTVSSASGGGGSGGRASGGGGSEIVMTQSPLSLPVTPGEPASISCRSSQSLLYSNGYNYVDWYLQKPGQSPQLLIYLGSNRASGVPDRFSGSGSGTDFKLQISRVEAEDVGIYYCMQGRQFPYSFGQGTKVEIK

SEQ ID NO: 47 - protein, 249 aa, synthetic construct
QVQLVQSGAEVRKTGASVKVSCKASGYIFDNFGINWVRQAPGQGLEWMGWINPKNNNTNYAQKFQGRVTITADESTNTAYMEVSSLRSEDTAVYYCARGPYYYQSYMDVWGQGTMVTVSSASGGGGSGGRASGGGGSDIVMTQTPLSLPVTPGEPASISCRSSQSLLHSNGYNYLNWYLQKPGQSPQLLIYLGSKRASGVPDRFSGSGSGTDFTLHITRVGAEDVGVYYCMQALQTPYTFGQGTKLEIK

SEQ ID NO: 48 - protein, 238 aa, synthetic construct
EVQLLETGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPATLSVSPGESATLSCRASQSVSSNLAWYQQKPGQAPRLLIYGASTRASGIPDRFSGSGSGTDFTLTISSLQAEDVAVYYCQQYGSSLTFGGGTKVEIK

SEQ ID NO: 49 - protein, 239 aa, synthetic construct
EVQLVETGGGLVQPGRSLRLSCAASGFTFDDYAMHWVRQAPGKGLEWVSGISWNSGSIGYADSVKGRFTISRDNAKNSLYLQMNSLRDEDTAVYYCARVGKAVPDVWGQGTTVTVSSGGGGSGGGGSGGGGSDIVMTQTPSSLSASVGDRVTITCRASQSISSYLNWYQQKPGKAPKLLIYAASSLQSGVPSRFSGSGSGTDFTLTISSLQPEDFATYYCQQSYSTPYSFGQGTRLEIK

SEQ ID NO: 50 - protein, 240 aa, synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDLRGAFDIWGQGTMVTVSSGGGGSGGGGSGGGGSSYVLTQSPSVSAAPGYTATISCGGNNIGTKSVHWYQQKPGQAPLLVIRDDSVRPSKIPGRFSGSNSGNMATLTISGVQAGDEADFYCQVWDSDSEHVVFGGGTKLTVL

SEQ ID NO: 51 - protein, 241 aa, synthetic construct
QVQLVQSGAEVKKPGASVKVSCKPSGYTVTSHYIHWVRRAPGQGLEWMGMINPSGGVTAYSQTLQGRVTMTSDTSSSTVYMELSSLRSEDTAMYYCAREGSGSGWYFDFWGRGTLVTVSSGGGGSGGGGSGGGGSSYVLTQPPSVSVSPGQTASITCSGDGLSKKYVSWYQQKAGQSPVVLISRDKERPSGIPDRFSGSNSADTATLTISGTQAMDEADYYCQAWDDTTVVFGGGTKLTVL

SEQ ID NO: 52 - protein, 241 aa, synthetic construct
EVQLVETGGGLVQPGGSLRLSCAASGITFSRYPMSWVRQAPGKGLEWVSGISDSGVSTYYADSAKGRFTISRDNSKNTLFLQMSSLRDEDTAVYYCVTRAGSEASDIWGQGTMVTVSSGGGGSGGGGSGGGGSEIVLTQSPATLSLSPGERATLSCRASQSVSNSLAWYQQKPGQAPRLLIYDASSRATGIPDRFSGSGSGTDFTLTISRLEPEDFAIYYCQQFGTSSGLTFGGGTKLEIK

SEQ ID NO: 53 - protein, 241 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVVRDGMDVWGQGTTVTVSSGGGGSGGGGSGGGGSEIVLTQSPATLSLSPGERATLSCRASQSVSSSYLAWYQQKPGQAPRLLIYGASSRATGIPDRFSGNGSGTDFTLTISRLEPEDFAVYYCQQYGSPPRFTFGPGTKVDIK

SEQ ID NO: 54 - protein, 242 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKIPQTGTFDYWGQGTLVTVSSGGGGSGGGGSGGGGSEIVLTQSPGTLSLSPGERATLSCRASQSVSSSYLAWYQQRPGQAPRLLIYGASSRATGIPDRFSGSGSGTDFTLTISRLEPEDFAVYYCQHYGSSPSWTFGQGTRLEIK

SEQ ID NO: 55 - protein, 243 aa, synthetic construct
EVQLLESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIVMTQTPLSLSVTPGQPASISCKSSQSLLRNDGKTPLYWYLQKAGQPPQLLIYEVSNRFSGVPDRFSGSGSGTDFTLKISRVEAEDVGAYYCMQNIQFPSFGGGTKLEIK

SEQ ID NO: 56 - protein, 243 aa, synthetic construct
QVQLQESGPGLVKPSETLSLTCTVSGGSISSSYYYWGWIRQPPGKGLEWIGSIYYSGSAYYNPSLKSRVTISVDTSKNQFSLRLSSVTAADTAVYYCARHWQEWPDAFDIWGQGTMVTVSSGGGGSGGGGSGGGGSETTLTQSPAFMSATPGDKVIISCKASQDIDDAMNWYQQKPGEAPLFIIQSATSPVPGIPPRFSGSGFGTDFSLTINNIESEDAAYYFCLQHDNFPLTFGQGTKLEIK

SEQ ID NO: 57 - protein, 244 aa, synthetic construct
VNLRESGPALVKPTQTLTLTCTFSGFSLRTSGMCVSWIRQPPGKALEWLARIDWDEDKFYSTSLKTRLTISKDTSDNQVVLRMTNMDPADTATYYCARSGAGGTSATAFDIWGPGTMVTVSSGGGGSGGGGSGGGGSDIQMTQSPSSLSASVGDRVTITCRASQDIYNNLAWFQLKPGSAPRSLMYAANKSQSGVPSRFSGSASGTDFTLTISSLQPEDFATYYCQHYYRFPYSFGQGTKLEIK

SEQ ID NO: 58 - protein, 244 aa, synthetic construct
EVQLLETGGGLVQPGGSLRLSCAASGFSFSSYAMSWVRQAPGKGLEWVSAISGSGGSTYYADSVKGRFTISRDNSKNTLYLQMNSLRAEDTAVYYCAKALVGATGAFDIWGQGTLVTVSSGGGGSGGGGSGGGGSEIVLTQSPGTLSLSPGERATLSCRASQSLSSNFLAWYQQKPGQAPGLLIYGASNWATGTPDRFSGSGSGTDFTLTITRLEPEDFAVYYCQYYGTSPMYTFGQGTKVEIK

SEQ ID NO: 59 - protein, 244 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVLWFGEGFDPWGQGTLVTVSSGGGGSGGGGSGGGGSDIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVDIK

SEQ ID NO: 60 - protein, 245 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARAGIAARLRGAFDIWGQGTMVTVSSGGGGSGGGGSGGGGSDIVMTQSPSSVSASVGDRVIITCRASQGIRNWLAWYQQKPGKAPNLLIYAASNLQSGVPSRFSGSGSGADFTLTISSLQPEDVATYYCQKYNSAPFTFGPGTKVDIK

SEQ ID NO: 61 - protein, 246 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAKTIAAVYAFDIWGQGTTVTVSSGGGGSGGGGSGGGGSEIVLTQSPLSLPVTPEEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 62 - protein, 246 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVEGSGSLDYWGQGTLVTVSSGGGGSGGGGSGGGGSEIVMTQSPGTLSLSPGERATLSCRASQSVSSAYLAWYQQKPGQPPRLLISGASTRATGIPDRFGGSGSGTDFTLTISRLEPEDFAVYYCQHYGSSFNGSSLFTFGQGTRLEIK

SEQ ID NO: 63 - protein, 246 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKMGWSSGYLGAFDIWGQGTTVTVSSGGGGSGGGGSGGGGSEIVLTQSPGTLSLSPGERATLSCRASQSVASSFLAWYQQKPGQAPRLLIYGASGRATGIPDRFSGSGSGTDFTLTISRLEPEDFAVYYCQHYGGSPRLTFGGGTKVDIK

SEQ ID NO: 64 - protein, 246 aa, synthetic construct
EVQLVESGGGLVQPGRSLRLSCTASGFTFDDYAMHWVRQRPGKGLEWVASINWKGNSLAYGDSVKGRFAISRDNAKNTVFLQMNSLRTEDTAVYYCASHQGVAYYNYAMDVWGRGTLVTVSSGGGGSGGGGSGGGGSEIVLTQSPGTLSLSPGERATLSCRATQSIGSSFLAWYQQRPGQAPRLLIYGASQRATGIPDRFSGRGSGTDFTLTISRVEPEDSAVYYCQHYESSPSWTFGQGTKVEIK

SEQ ID NO: 65 - protein, 248 aa, synthetic construct
EVQLQQSGPGLVKPSQTLSLTCAISGDSVSSNSAAWNWIRQSPSRGLEWLGRTYYRSKWYSFYAISLKSRIIINPDTSKNQFSLQLKSVTPEDTAVYYCARSSPEGLFLYWFDPWGQGTLVTVSSGGDGSGGGGSGGGGSSSELTQDPAVSVALGQTIRITCQGDSLGNYYATWYQQKPGQAPVLVIYGTNNRPSGIPDRFSASSSGNTASLTITGAQAEDEADYYCNSRDSSGHHLLFGTGTKVTVL

SEQ ID NO: 66 - protein, 248 aa, synthetic construct
EVQLVETGGGLVQPGGSLRLSCAASGFTFSSYAMSWVRQAPGKGLEWVSAISGSGGSTYYADSVKGRFTMSRENDKNSVFLQMNSLRVEDTGVYYCARANYKRELRYYYGMDVWGQGTMVTVSSGGGGSGGGGSGGGGSEIVMTQSPGTLSLSPGESATLSCRASQRVASNYLAWYQHKPGQAPSLLISGASSRATGVPDRFSGSGSGTDFTLAISRLEPEDSAVYYCQHYDSSPSWTFGQGTKVEIK

SEQ ID NO: 67 - protein, 248 aa, synthetic construct
EVQLVETGGGLVQPGGSLRLSCAASGFTFSSYAMSWVRQAPGKGLEWVSAISGSGGSTYYADSVKGRFTISRDNSKNTLYLQMNTLKAEDTAVYYCARATYKRELRYYYGMDVWGQGTTVTVSSGGGGSGGGGSGGGGSEIVLTQSPSTLSLSPGESATLSCRASQSVSTTFLAWYQQKPGQAPRLLIYGSSNRATGIPDRFSGSGSGTDFTLTIRRLEPEDFAVYYCQQYHSSPSWTFGQGTKVEIK

SEQ ID NO: 68 - protein, 248 aa, synthetic construct
QVQLVESGGGLVQPGGSLRLSCAASGFTFSSYAMSWVRQAPGKGLEWVSAISGSGGSTYYADSVKGRFTISRDNAKNSLYLQMNSLRAEDTAIYYCARATYKRELRYYYGMDVWGQGTMVTVSSGGGGSGGGGSGGGGSEIVMTQSPGTVSLSPGERATLSCRASQSVSSSFLAWYQQKPGQAPRLLIYGASSRATGIPDRFSGSGSGTDFTLTISRLEPEDSAVYYCQQYHSSPSWTFGQGTRLEIK

SEQ ID NO: 69 - protein, 249 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 70 - protein, 249 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 71 - protein, 251 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGYDSSGYYRDYYGMDVWGQGTTVTVSSGGGGSGGGGSGGGGSEIVLTQSPGTLSLSPGERATLSCRASQSVSSSYLAWYQQKPGQAPRLLIYGTSSRATGISDRFSGSGSGTDFTLTISRLEPEDFAVYYCQHYGNSPPKFTFGPGTKLEIK

SEQ ID NO: 72 - protein, 253 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGGYQLLRWDVGLLRSAFDIWGQGTMVTVSSGGGGSGGGGSGGGGSSYVLTQPPSVSVAPGQTARITCGGNNIGSKSVHWYQQKPGQAPVLVLYGKNNRPSGVPDRFSGSRSGTTASLTITGAQAEDEADYYCSSRDSSGDHLRVFGTGTKVTVL

SEQ ID NO: 73 - protein, 218 aa, synthetic construct
QLQLQESGPGLVKPSETLSLICTVSGGSISSSRYYWDWIRQPPGKGLEWIGSLYYSGNTYYKPSLKSRVTISVDTSKNQFSLKLTSVTADTAVYYCARQGATHALDLWGPGTMVTVSSGGGGSGGGGSGGGGSEIVITQSPATLSLSPGERATLSCGASQSVTSSYLAWYQQKPGLAPPLLIYDASGRATGIPDRISGSGSGTDESITISRLEPEDFA

SEQ ID NO: 74 - protein, 244 aa, synthetic construct
QVQLVQSGAEVKKPGASVKVSCKASGYTFSNNGFSWVRQAPGQGLEWMGWISGFNGKTYYTKTLQGRVTMTIDTSTSTAYMDLRSLRSDDTAVYYCARGLLLSGELWGFDYWGQGTLVTVSSGGGGSGGGGSGGGGSDIQLTQSPSFLSASVGDRVTITCRASQDISSFLAWYQQKPGKAPKLLIFAASTLQSGVPSRISGSGSGTEFTLTISSLQPEDFATYYCQQFNSYPRTFGQGTKVEIK

SEQ ID NO: 75 - protein, 244 aa, synthetic construct
DIQLTQSPSFLSASVGDRVTITCRASQDISSFLAWYQQKPGKAPKLLIFAASTLQSGVPSRISGSGSGTEFTLTISSLQPEDFATYYCQQFNSYPRTFGQGTKVEIKGGGGSGGGGSGGGGSQVQLVQSGAEVKKPGASVKVSCKASGYTFSNNGFSWVRQAPGQGLEWMGWISGFNGKTYYTKTLQGRVTMTIDTSTSTAYMDLRSLRSDDTAVYYCARGLLLSGELWGFDYWGQGTLVTVSS

SEQ ID NO: 76 - protein, 247 aa, synthetic construct
QVQLVESGGGVVQPGRSLRLSCAASGFTFSSYGMHWVRQAPGKGLEWVAVISYDGRNKNYADSVKGRFTISRDNSKNTLYLQMNSLRAEDTAVYYCAREGEATYYDILTGPFDYWGQGTLVTVSSGGGGSGGGGSGGGGSDIQMTQSPSSLSASVGDRITITCRASQDIRNYLGWYQQKPGKAPKVLIFAASSLQSGVPSRFSGSGSGTDFTLTISSLQPEDFATYYCLQDYIYPWTFAQGTKVEIK

SEQ ID NO: 77 - protein, 247 aa, synthetic construct
DIQMTQSPSSLSASVGDRITITCRASQDIRNYLGWYQQKPGKAPKVLIFAASSLQSGVPSRFSGSGSGTDFTLTISSLQPEDFATYYCLQDYIYPWTFAQGTKVEIKGGGGSGGGGSGGGGSQVQLVESGGGVVQPGRSLRLSCAASGFTFSSYGMHWVRQAPGKGLEWVAVISYDGRNKNYADSVKGRFTISRDNSKNTLYLQMNSLRAEDTAVYYCAREGEATYYDILTGPFDYWGQGTLVTVSS

SEQ ID NO: 78 - protein, 247 aa, synthetic construct
QVQLVESGGGVVQPGRSLRLSCAASGFTFSSYGMHWVRQAPGKGLEWVAVISYDGRNKNYADSVKGRFTISRDNSKNTLYLQMNSLRAEDTAVYYCAREGEATYYDILTGPFDYWGQGTLVTVSSGGGGSGGGGSGGGGSDIQLTQSPSFLSASVGDRVSITCRASQGISSYLAWYQQKPGKAPKVLIYVASTLQSGVPSRFSGSGSGTEFTLTISSLQPDDFATYYCQQLYSYPRAFGQGTKVEIK

SEQ ID NO: 79 - protein, 250 aa, synthetic construct
QVQLVQSGAEVKKPGASVKVSCKGSGYTFSTYGIGWVRQAPGQGLEWLAWISAYSGKTNYAQKVQGRVTLTTDTSTNTAYMELRSLRSDDTAVYYCARDDGAGTDYYYGMDVWGQGTTVTVSSGGGGSGGGGSGGGGSDIVMTQTPLSLSVTPGQPASISCKSSQSLLYSDGKTFLYWYLQKPGQPPQLLIYEGSNRFSGVPDRFSGSGSGTDFTLKISRVEAEDVGVFYCMQSIQLPNTFGQGTKLEIK

SEQ ID NO: 80 - protein, 250 aa, synthetic construct
DIVMTQTPLSLSVTPGQPASISCKSSQSLLYSDGKTFLYWYLQKPGQPPQLLIYEGSNRFSGVPDRFSGSGSGTDFTLKISRVEAEDVGVFYCMQSIQLPNTFGQGTKLEIKGGGGSGGGGSGGGGSQVQLVQSGAEVKKPGASVKVSCKGSGYTFSTYGIGWVRQAPGQGLEWLAWISAYSGKTNYAQKVQGRVTLTTDTSTNTAYMELRSLRSDDTAVYYCARDDGAGTDYYYGMDVWGQGTTVTVSS

SEQ ID NO: 81 - protein, 244 aa, synthetic construct
QVQLVQSGAEVKKPGASVKVSCKASGYTFSNNGFSWVRQAPGQGLEWMGWISGFNGKTYYTKTLQGRVTMTIDTSTSTAYMDLRSLRSDDTAVYYCARGLLLSGELWGFDYWGQGTLVTVSSGGGGSGGGGSGGGGSDIQLAQSPSFLSASVGDRVTITCRASQDISSFLAWYQQKPGKAPKLLIFAASTLQSGVPSRISGSGSGTEFTLTISSLQPEDFATYYCQQFNSYPRTFGQGTKVEIK

SEQ ID NO: 82 - protein, 244 aa, synthetic construct
DIQLAQSPSFLSASVGDRVTITCRASQDISSFLAWYQQKPGKAPKLLIFAASTLQSGVPSRISGSGSGTEFTLTISSLQPEDFATYYCQQFNSYPRTFGQGTKVEIKGGGGSGGGGSGGGGSQVQLVQSGAEVKKPGASVKVSCKASGYTFSNNGFSWVRQAPGQGLEWMGWISGFNGKTYYTKTLQGRVTMTIDTSTSTAYMDLRSLRSDDTAVYYCARGLLLSGELWGFDYWGQGTLVTVSS

SEQ ID NO: 83 - protein, 243 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 84 - protein, 243 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 85 - protein, 244 aa, synthetic construct
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 86 - protein, 244 aa, synthetic construct
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 87 - protein, 243 aa, synthetic construct
DIVMTQSQRFMSTSVGDRVSITCKASQSVGTAVAWYQQTPGQFPKLLIYSTSNRYTGVPDRFTGSGSGTDFTLTISNMQSEDLADYFCQQYSTYPLTFGSGTKLELKGGGGSGGGGSGGGGSEVQLQQSGPELVKPGASMKISCKASDYSFTDYIMTWVKQSHGKNLEWIGLINPYNGGTTYNQKFKDKATFTVDKSSTTAYMDLLSLTSEDSAVYYCARRGITTDYYTMDYWGQGTSVTVSS

SEQ ID NO: 88 - protein, 246 aa, synthetic construct
DIVMTQSPSSLAMSVGQKVTMSCKSSQSLLNSSIQKNYLAWYQQKPGQSPKLLVYFASTRESGVPDRFIGSGSGTDFTLTISSVQAEDLADYFCQQHYSSPLTFGAGTKLELKGGGGSGGGGSGGGGSEVQLQQSGPVLVKPGASVKMSCKASGYTFTDLYMNWVKQSHGKSLEWIGVINPYNGGTSYNQKFKAKATLTVDKSSITAYMELNSLTSEDSAVYYCARGDSIYVMDYWGQGTSVTVSS

SEQ ID NO: 89 - protein, 244 aa, synthetic construct
DIVMTPSQKFMSTSVGDRVSVTCKASQNVGTNVAWYQQKPGQSPKALIYSASYRYSGVPDRFTGSGSGTDFTLTISNVQSEDLAEYFCQHYNSYPFTFGSGTKLEIKGGGGSGGGGSGGGGSEVKLLQSGGGLVQPGGSLKLSCAASGIDFSRYWMSWVRRAPGKGLEWIGEINPDSSTINYAPSLKDKFIISRDAAKNTLYLQMSKVRSEDTALYYCATLYYDYDGDYAMDYWGQGTSVTVSS

SEQ ID NO: 90 - protein, 246 aa, synthetic construct
DIVMTQSPSSLAMSVGQKVTMSCKSSQSLLNSSIQKNYLAWYQQKPGQSPKLLIYFASTRESGVPDRFIGSGSGTDFTLTISSVQAEDLADYFCQQHYSTPLTFGAGTKLELKGGGGSGGGGSGGGGSEVQLQQSGPVLVKPGASVKMSCKASGYTFTDLYMNWLKQSHGKRLEWIGVINPYNGGTSYNQKFKGKATLTVDKSSSTAYMDLNSLTSEDSAVYYCARGDSIYVMDYWGQGTSFTVSS

SEQ ID NO: 91 - protein, 240 aa, synthetic construct
ENVLTQSPAIMSASPGEKVTMTCSASSSVSYMHWYQQKSSTSPKLWIYDTSKLSSGVPGRFSGSGSGKSYSLTISSMEAEDVATYYCFQGSGYPLFTFGSGTKLEIKGGGGSGGGGSGGGGSEVQLQQSGPVLVKPGASVKMSCKASGYTFTDLYMNWLKQSHGKRLEWIGVINPYNGGTSYNQKFKGKATLTVDKSSSTAYMDLNSLTSEDSAVYYCARGDSIYVMDYWGQGTSFTVSS

SEQ ID NO: 92 - protein, 246 aa, synthetic construct
DIVMTQSPSSLALSVGQKVTMSCKSSQSLLDNSNQKHYLAWYQQKPGQSPKLLVYFASTRESGVPDRFIGSGSGTDFTLTISSVQAEDLADYFCQQHYTAPLTFGAGTKLALKGGGGSGGGGSGGGGSEVQLQQSGPVLVKPGASVKMSCKVSGYTFTDYYMNWVKQSHGKSLEWIGVITPYNGANRYNQKFKGKATLTVDKSSSTAYMEVSSLTSEDSAVYYCARGDSIYVMDYWGQGTSVIVSS

SEQ ID NO: 93 - protein, 246 aa, synthetic construct
DIVMTQSPSSLAMSVGQKVTMSCKSSQSLLNSSIQKNYLAWYQQKPGQSPKLLVYFASTRESGVPDRFIGSGSGTDFTLTISSVQAEDLADYFCQQHYSTPLTFGGGTKLELKGGGGSGGGGSGGGGSEVQLQQSGPVLVKPGASVKMSCKASGYTFTDYSLNWVKQSHGKSLEWIGVVNPYNGGTSHNQKFKGKATLTVDKSSSTAYMELNSLTSEDSAVYYCARPDSIYVMDYWGQGTSVTVSS

SEQ ID NO: 94 - protein, 246 aa, synthetic construct
DIVMTQSPSSLAMSVGQKVTMSCKSSQSLLNSNIQKNYLAWYQQKPGQSPKLLVYFASTRESGVPDRFIGSGSGTDFTLTISSVQAEDLADYFCQQHYSTPLTFGAGTKLELKGGGGSGGGGSGGGGSEVQLQQSGPVLVKPGASVKMSCKASGYTFTDYSLNWVKQSHGKSLEWIGVVNPYNGGTTYNQKFKGKATLTVDKSSSTAYMELNSLTSEDSAVYYCARPDSIYVMDSWGQGTSVTVSS

SEQ ID NO: 95 - protein, 239 aa, synthetic construct
DIKMTQSPSSMYVSLGERVTITCKASQDINRNLSWFQQKPGKSPKTLIYRANRLVDGVPLRFSGSGSGQDYSLTISSLEYEDMGIYYCLQYDEFPRTFGGGTKLEIKGGGGSGGGGSGGGGSQVTLKESGPGILQSSQTLSLTCSFSGFSLNTSGMGVNWIRQSSGKDLEWLAHIYWNDDKRYNPSLKSRLTISKDTSRNQVFLRITSVDATDTATYFCCRSRLSFDYWGHGTTLTVSS

SEQ ID NO: 96 - protein, 267 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 97 - protein, 242 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 98 - protein, 242 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 99 - protein, 242 aa, synthetic construct
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 100 - protein, 242 aa, synthetic construct
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 101 - protein, 242 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 102 - protein, 242 aa, synthetic construct
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 103 - protein, 247 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 104 - protein, 247 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 105 - protein, 247 aa, synthetic construct
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 106 - protein, 247 aa, synthetic construct
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 107 - protein, 247 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 108 - protein, 247 aa, synthetic construct
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 109 - protein, 241 aa, synthetic construct
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 110 - protein, 242 aa, synthetic construct
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKRSGGGGSGGGGSGGGGSQVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 111 - protein, 250 aa, synthetic construct
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRSGGGGSGGGGSGGGGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSS

SEQ ID NO: 112 - protein, 272 aa, synthetic construct
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 113 - protein, 256 aa, synthetic construct
DIQMTQSPSTLSASVGDRVTITCRASQSISSWLAWYQQKPGKAPKLLIYKASSLESGVPPRFSGSGSGTEFTLTISSLQPDDFATYYCQQYNSAYTFGQGTKLEIKGSGGGGSGGGGSGGGGSQVQLVESGGGVVQPGRSLRLSCAASGFTFSRHGMHWVRQAPGKGLEWVAVIWYDGSNQYYVDSVKGRFTISRDNSKNTLDLQMNSLRVEDTAVYYCARRSITWYGGFDIWGQGTMVTVSSAQTTAPSVYPLAP

SEQ ID NO: 114 - protein, 259 aa, synthetic construct
DIQMTQSPSTLSASVGDRVTITCRASQSISSWLAWYQQKPGKAPKLLIYKASSLESGVPPRFSGSGSGTEFTLTISSLQPDDFATYYCQQYNSAYTFGQGTKLEIKGGGGSGGGGSGGGGSGGGGSQVQLVESGGGVVQPGRSLRLSCAASGFTFSRHGMHWVRQAPGKGLEWVAVIWYDGSNQYYVDSVKGRFTISRDNSKNTLDLQMNSLRVEDTAVYYCARRSITWYGGFDIWGQGTMVTVSSAQTTAPSVYPLAP

SEQ ID NO: 115 - protein, 257 aa, synthetic construct
DIQMTQSPSTLSASVGDRVTITCRASQSISSWLAWYQQKPGKAPKLLIYKASSLESGVPPRFSGSGSGTEFTLTISSLQPDDFATYYCQQYNSAYTFGQGTKLEIKGTSSGSGKPGSGEGSTKGQVQLVESGGGVVQPGRSLRLSCAASGFTFSRHGMHWVRQAPGKGLEWVAVIWYDGSNQYYVDSVKGRFTISRDNSKNTLDLQMNSLRVEDTAVYYCARRSITWYGGFDIWGQGTMVTVSSAQTTAPSVYPLAP

SEQ ID NO: 116 - protein, 127 aa, synthetic construct
QVQLQESGGGSVQAGGSLRLSCAVRRPTDSRNCMAWFRQAPGEQREAVAGIDIYKTTGYAESVKGRFTISQDNAKNTLFLQMNSLKPEDSGTYYCAAARPCKYGSEWRRSASDFLYWGQGTQVTVSS

SEQ ID NO: 117 - protein, 109 aa, synthetic construct
EVQLLESGGGLVQPGGSLRSCEASGFNAMTSIDSWTDAVKGWVRQPPGKGLEWVSRFAISQDNAKNTVYLQMNSLKPEDTAMYYCALSKCYTRVYDYWGQGTQVTVSSG

SEQ ID NO: 118 - protein, 104 aa, synthetic construct
EVQLQESGGGLVQPGGSLRLSCAASGFIYMVGIKTERDGVKGWVRQAPGKGLEWLSRFTIPRDNAKNTLYLQMNNLKSEDTALYYCATEENDWGQGTQVTVSSG

SEQ ID NO: 119 - protein, 284 aa, synthetic construct
MVLLVTSLLLCELPHPAFLLIPDTEVQLVESGGGLVKPGGSLKLSCAASGFAFSIYDMSWVRQTPEKRLEWVAYISSGGGTYYPDTVKGRFTISRDNAKNTLYLQMSSLKSEDTAMYYCARHSGYGTHWGVLFAYWGQGTLVTVSAGGGGSGGGGSGGGGSDIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIKAKTTPPSVYGRVKDPK

SEQ ID NO: 120 - protein, 236 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 121 - protein, 480 aa, synthetic construct
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVAL

SEQ ID NO: 122 - protein, 237 aa, synthetic construct
QVTLKESGPGILQPSQTLSLTCSFSGFSLSTSGMGVGWFRQPSGKGLEWLAHIWWDDDKRYNPALKNRLTISKDTSSNQVFLKIASVDTADTATYYCARITISYDGYSYYFDYWGQGTTLTVSSLINKERDIQLTQCPASLSASVGETVTITCRASENIYSYLAWYQQKQGKSPQFLVYNAKTLAEGVPSRFSGSGSGTQFSLKINSLQPEDFGSYYCQHHYGTPYTFGGGTKLEIK

SEQ ID NO: 123 - protein, 237 aa, synthetic construct
DIQLTQCPASLSASVGETVTITCRASENIYSYLAWYQQKQGKSPQFLVYNAKTLAEGVPSRFSGSGSGTQFSLKINSLQPEDFGSYYCQHHYGTPYTFGGGTKLEIKLINKERQVTLKESGPGILQPSQTLSLTCSFSGFSLSTSGMGVGWFRQPSGKGLEWLAHIWWDDDKRYNPALKNRLTISKDTSSNQVFLKIASVDTADTATYYCARITISYDGYSYYFDYWGQGTTLTVSS

SEQ ID NO: 124 - protein, 234 aa, synthetic construct
DIQLTQCPASLSASVGETVTITCRASENIYSYLAWYQQKQGKSPQFLVYNAKTLAEGVPSRFSGSGSGTQFSLKINSLQPEDFGSYYCQHHYGTPYTFGGGTKLEIKGGGGSGGGGSGGGGSGGGGSDIQLTQCPASLSASVGETVTITCRASENIYSYLAWYQQKQGKSPQLLVYNAKTLAEGVPSRFSGSGSGTQFSLKINSLQPEDFGSYYCQHHYGTPFTFGSGTKLEIK

SEQ ID NO: 125 - protein, 234 aa, synthetic construct
DIQLTQCPASLSASVGETVTITCRASENIYSYLAWYQQKQGKSPQLLVYNAKTLAEGVPSRFSGSGSGTQFSLKINSLQPEDFGSYYCQHHYGTPFTFGSGTKLEIKGGGGSGGGGSGGGGSGGGGSDIQLTQCPASLSASVGETVTITCRASENIYSYLAWYQQKQGKSPQFLVYNAKTLAEGVPSRFSGSGSGTQFSLKINSLQPEDFGSYYCQHHYGTPYTFGGGTKLEIK

SEQ ID NO: 126 - protein, 245 aa, synthetic construct
EVQLQQSGPELVKPGASVKISCKASGYTFTDYNIHWVKQSHGKSLEWIGYIYPYNGGTGYNQKFKSKATLTVHNSSSTAYMELRSLTSEDSAVYYCARLYYYGSSSYWGQGTTLTVSSGGGGSGGGGSGGGGSGGGGSDIVMTQSQKFMSTSVGDRVSVTCKASQNVGTNVAWYQQKPGQSPKALIYSASYRNSGVPDRFTGSGSGTDFTLTISNVQSEDLAEYFCQQYNSYPYTFGGGTKLEIK

SEQ ID NO: 127 - protein, 245 aa, synthetic construct
DIVMTQSQKFMSTSVGDRVSVTCKASQNVGTNVAWYQQKPGQSPKALIYSASYRNSGVPDRFTGSGSGTDFTLTISNVQSEDLAEYFCQQYNSYPYTFGGGTKLEIKGGGGSGGGGSGGGGSGGGGSEVQLQQSGPELVKPGASVKISCKASGYTFTDYNIHWVKQSHGKSLEWIGYIYPYNGGTGYNQKFKSKATLTVHNSSSTAYMELRSLTSEDSAVYYCARLYYYGSSSYWGQGTTLTVSS

SEQ ID NO: 128 - protein, 248 aa, synthetic construct
DVKLQESGPGLVKPSQSLSLTCSVTGYSISSGYYWNWIRQFPGNKLEWMGYISYDGSNNYNPSLKNRISITRDTSKNQFFLKLNSVTIEDTATYHCARGWSWFAYWGQGTLVTVSAGGGGSGGGGSGGGGSGGGGSDVLMTQTPLSLPVSLGDQASISCRSSQSLVLSNGNTYLHWYLQKPGQSPKLLIYKVSNRFSGVPDRFSGSGSGTDFTLKISRVEAEDLGLYFCSQSTHFPWTFGGGTKLEIK

SEQ ID NO: 129 - protein, 248 aa, synthetic construct
DVLMTQTPLSLPVSLGDQASISCRSSQSLVLSNGNTYLHWYLQKPGQSPKLLIYKVSNRFSGVPDRFSGSGSGTDFTLKISRVEAEDLGLYFCSQSTHFPWTFGGGTKLEIKGGGGSGGGGSGGGGSGGGGSDVKLQESGPGLVKPSQSLSLTCSVTGYSISSGYYWNWIRQFPGNKLEWMGYISYDGSNNYNPSLKNRISITRDTSKNQFFLKLNSVTIEDTATYHCARGWSWFAYWGQGTLVTVSA

SEQ ID NO: 130 - protein, 249 aa, synthetic construct
QVQLKESGPGLVQPSQSLSITCTVSGFSLTIYGVHWIRQSPGKGLEWLGVMWSGGSTDYNAAFISRLSISKDNSKSQVFFKMNSLQANDTAIYYCARNDGYYWFAYWGQGTLVTVSAGGGGSGGGGSGGGGSGGGGSDVLMTQTPLSLPVSLGDQASISCRSSQSLVHNNGNTYLHWYLQKPGQSPKLLIYKVSNRFSGVPDRFSGSGSGTDFTLKISRVEAEDLGVYFCSQSTHVPYSFGGGTKLEIK

SEQ ID NO: 131 - protein, 249 aa, synthetic construct
DVLMTQTPLSLPVSLGDQASISCRSSQSLVHNNGNTYLHWYLQKPGQSPKLLIYKVSNRFSGVPDRFSGSGSGTDFTLKISRVEAEDLGVYFCSQSTHVPYSFGGGTKLEIKGGGGSGGGGSGGGGSGGGGSQVQLKESGPGLVQPSQSLSITCTVSGFSLTIYGVHWIRQSPGKGLEWLGVMWSGGSTDYNAAFISRLSISKDNSKSQVFFKMNSLQANDTAIYYCARNDGYYWFAYWGQGTLVTVSA

SEQ ID NO: 132 - protein, 253 aa, synthetic construct
QVKLQQSGAELVRPGASVKLSCKASGYTFTSYWMNWVKQRPEQGLEWIGRIDPYDSETHYNQKFKDKAILTVDKSSSTAYMQLSSLTSEDSAVYYCARREYGNYFYALDYWGQGTSVTVSSGGGGSGGGGSGGGGSGGGGSDIVMTQTPLSSCRSSQSLVHSNGNTYLHWYLQKPGQSPKLLIYKVSNRFSGVPDRFSGSGSGTDFTLKISRVEAEDLGVYFCSQSTHVPYTFGGGTKLPVSLGDQASILEIK

SEQ ID NO: 133 - protein, 253 aa, synthetic construct
DIVMTQTPLSSCRSSQSLVHSNGNTYLHWYLQKPGQSPKLLIYKVSNRFSGVPDRFSGSGSGTDFTLKISRVEAEDLGVYFCSQSTHVPYTFGGGTKLPVSLGDQASILEIKGGGGSGGGGSGGGGSGGGGSQVKLQQSGAELVRPGASVKLSCKASGYTFTSYWMNWVKQRPEQGLEWIGRIDPYDSETHYNQKFKDKAILTVDKSSSTAYMQLSSLTSEDSAVYYCARREYGNYFYALDYWGQGTSVTVSS

SEQ ID NO: 134 - protein, 128 aa, synthetic construct
QVQLVESGGGVVRPGDSLRLSCTVSGFTLDYYVMGWFRQAPGKEREVVSCISSSDGVTYYADSVKGRFTISRDNAKNTVYLQMNSLKPEDTAIYYCAVDKPFYDGGYYYTCPVDFDSWGQGTQVTVSS

SEQ ID NO: 135 - protein, 110 aa, synthetic construct
QVQLVESGGGLVQAGGSLSVSCTASESTFSIMMGWFRQAPGKQREMVAVINSAGSTNYADSAEGRFTISRDLAKKTVSLQMNSLKPEDTAVYYCYVDGYFGQGTQVTVSS

SEQ ID NO: 136 - protein, 115 aa, synthetic construct
QVKLEESGGGLVQAGGSLRLSCAASGSTFSSSVMNWYRQAPGKQRELVAVITSSGSTHYADSVKGRFTISRDNAKNTVNLQMNSLKPEDTAVYYCNAQYGRRSDWGQGTQVTVSS

SEQ ID NO: 137 - protein, 127 aa, synthetic construct
QVKLEESGGGLVQPGGSLRLSCTVSGVTFDYYVMGWFRQAPGKEREVVSCMRNSDGVTYYADSVKGRFTISRDNAKNTVYQMNSLKPDDTAIYYCAVDKPFYDGGNYYTCPVDFDSWGQGTQVTVSS

SEQ ID NO: 138 - protein, 118 aa, synthetic construct
QVKLEESGGGLVQAGGSLRLSCAASGSISSINAMGWYRQVPGKQRELVAIITTAGNTRYGDSVKGRFTISRDNARKTVYLQMNSLKPEDTAVYYCAGKLYVNKEYTYWGQGTQVTVSS

SEQ ID NO: 139 - protein, 127 aa, synthetic construct
QVQLVESGGGLVQPGGSLRLSCTVSGFSLDYYVMGWFRQAPGKEREVSCISSSDGATYYPDSVKGRFTISRDNAKKTVYLQMNALKPEDTAIYYCAVDKPFYDGGSYYTCPVDFGSWGQGTQVTVSS

SEQ ID NO: 140 - protein, 128 aa, synthetic construct
QVQLVESGGGLVQPGDSLRLSCTVSGFTSDYYAMGWFRQAPGKEREAVSCISSSDGVTYYADSVRGRFTISRDNAKNTVYLEMNSLKPEDTAIYYCAVDKPFYDGGYLYTCPVDFDSWGQGTQVTVSS

SEQ ID NO: 141 - protein, 114 aa, synthetic construct
QVQLVESGGGLVQAGGSLRLSCAASGSTFSLKAMAWYRQAPGKQRERVGVINSDGSYTTDAASVQGRFTISRDNAKNTVYLQMNSLKPEDTAVYYCNTMPPWPWGQGTQVTVSS

SEQ ID NO: 142 - protein, 116 aa, synthetic construct
QVQLVESGGGLVQPGGSLRLSCAASGSIFRITAVMNWYRQAPGKERELVAVITSAGETHYAESVKGRFTISRDNAKNTMYLQMNSLKPEDTAVYYCNAKWGQYEHWGQGTQGTVSS

SEQ ID NO: 143 - protein, 118 aa, synthetic construct
QVQLVESGGGLVQAGGSLRLSCSASGITSSINSMGWHRQAPGKQRELVASIGRGGSGSTKYADSGKGRFLISRDNNKNILFLEMNSLKPEDTADYYCLEVTTDLSSYWGRGTQVTVSS

SEQ ID NO: 144 - protein, 120 aa, synthetic construct
QVQLVESGGGLVQAGGSLRLSCAVSGRIFRSYVLGWFRQAPGKERELVARIGWSDTYYADSVKGRFTISRDNAKNTVDLQMNSLKPEDTAVYYCAANSPPYGPQRDEFGYWGQGTQVTVS

SEQ ID NO: 145 - protein, 127 aa, synthetic construct
QVKLEESGGGLVQPGGSLRLSCTVSGFTLDSYVMGWFRQAPGKEREAVSCISSSDGVTYYADSVKGRFTISRDNAKNTYLQMNSLKPEDTAIYYCAVDKPFYDGGYLYTCPVDFDSWGQGIQVTVSS

SEQ ID NO: 146 - protein, 115 aa, synthetic construct
QVKLEESGGGLGQAGGAVRLSCADSGSIFRIAVMNWYRQAPGKERELVAVITIAGETHYAYSVKGQFTISRYNAKNTMYLQMNRLKPEDTAVYYCYANGGRDEYWGRGTQVTVSS

SEQ ID NO: 147 - protein, 121 aa, synthetic construct
QVKLEESGGGLGQAGGSLRLSCAVSGRISRSYVLGWFRQAPGKERELVARIGCSDTYYADSAKGRFTISRNNADNTVDLQMNSLKPEDTAVYYCAEYSPPYGPQRDEFDYWGQGTQVTVSS

SEQ ID NO: 148 - protein, 118 aa, synthetic construct
QVQLVESGGGLVQAGGSLRLSCAASGSTFSLNTMAWYRQAPGNQREYVAAASSDGYTNYADSVRGRFTISRDNDKNTMYLQMNSLRPEDTAVYYCTWGTGRFADYIYWGQGTQVTVSS

SEQ ID NO: 149 - protein, 126 aa, synthetic construct
QVQLVESGGGLVQAGGSLRLSCAASGGTFSVYTMAWFRQAPGKEREFVAAIRGSGGTYYRDSVKGRFTISRDNAKNTVYLQMNSLKPEDTAVYYCAVRIRRTLVEPLTKETLYDYWGQGTQVTVSS

SEQ ID NO: 150 - protein, 127 aa, synthetic construct
QVKLEESGGGLVQPGGSLRLSCTVSGRTSSVYGMAWFRQTPGKEREFVAAMSWSGGPTYFADSVKGRFGSSRDNAKNTVYLQMNGLRPEDTAVYYCAVRIRRTLLEPLTKETLYDYWGQGTQVTVSS

SEQ ID NO: 151 - protein, 115 aa, synthetic construct
QVQLVESGGGLVQPGGSLRLSCAASGSIFRITVMNWHRQAPGKERELSGVITSGGDSHYAASVKGRFTIYRDNTKNTMYLQMNRLKPEDTDYYYCNAKWGGDEYWGQGTHDTGSS

SEQ ID NO: 152 - protein, 248 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGSGGGGSGGGGSGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 153 - protein, 251 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGGGSGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 154 - protein, 249 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGTSSGSGKPGSGEGSTKGQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 155 - protein, 121 aa, synthetic construct
EVQLVESGGDSVQPGGSLRLSCAVSGDTHSSYCLAWFRQAPGKEREGVAFVDSDGKQIHADSVKGRFTGSRDNTKNTLFLQMDSLQLEDTAMYYCAAAPWCYREAEDFTIWGQGTQVTVSS

SEQ ID NO: 156 - protein, 127 aa, synthetic construct
EVQLVESGGASVQAGGSLTLSCAASGYTNSRRYMAWFRQTPGKEREGVAYIYTGDGDSRTYYADSVKGRFTISRDNAKGTVNLQMNSLQPGDSAMYYCAADVWYHGDWNDPKLYPYWGQGTQVTVSS

SEQ ID NO: 157 - protein, 240 aa, synthetic construct
QVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 158 - protein, 244 aa, synthetic construct
QVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGPSSWGEFDYWGQGTLVTVSSASGGGGSGGGGSGGGGSDIRLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 159 - protein, 245 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKETDYYGSGTFDYWGQGTLVTVSSASGGGGSGGGGSGGGGSDIQMTQSPSSLSASVGDRVTISCRASQGIGIYLAWYQQRSGKPPQLLIHGASTLQSGVPSRFSGSGSGTDFTLTISSLQPEDFASYWCQQSNNFPPTFGQGTKVEIK

SEQ ID NO: 160 - protein, 245 aa, synthetic construct
QVQLVQSGAEVKKPGASVRVSCKASGYMFTDFFIHWVRQAPGQGLEWMGWINPNSGVTKYAQKFQGRVTMTRNTSISTAYMELSSLRSEDTAVYYCATWYSSGWYGIANIWGQGTMVTVSSASGGGGSGGGGSGGGGSDIQLTQSPSSLSASVGDRVTITCQASHDISNYLHWYQQKPGKAPKLLIYDASNLETGVPSRFTGSGSGTDFTLTIRSLQPEDVAAYYCQQSDDLPHTFGQGTKVDIK

SEQ ID NO: 161 - protein, 246 aa, synthetic construct
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 162 - protein, 246 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGGGGSGGGGSSGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 163 - protein, 248 aa, synthetic construct
QVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 164 - protein, 249 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKEDTIRGPNYYYYGMDVWGQGTTVTVSSASGGGGSGGGGSGGGGSETTLTQSPSSVSASVGDRVSITCRASQDIDTWLAWYQLKPGKAPKLLMYAASNLQGGVPSRFSGSGSGTDFILTISSLQPEDFATYYCQQASIFPPTFGGGTKVDIK

SEQ ID NO: 165 - protein, 250 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGGSLPDYGMDVWGQGTMVTVSSASGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLITFGQGTKVDIK

SEQ ID NO: 166 - protein, 250 aa, synthetic construct
QVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIK

SEQ ID NO: 167 - protein, 251 aa, synthetic construct
QVQLVQSGAEVKKPGASVRVSCKASGYIFTNYYVHWVRQAPGQGLEWMGIISPSGGSPTYAQRLQGRVTMTRDLSTSTVYMELSSLTSEDTAVYFCARESRLRGNRLGLQSSIFDHWGQGTLVTVSSASGGGGSGGGGSGGGGSDIRMTQSPPSLSASVGDRVTIPCQASQDINNHLNWYQQKPGKAPQLLIYDTSNLEIGVPSRFSGSGSGTDFTLTISSLQPEDIATYYCQQYENLPLTFGGGTKVEIK

SEQ ID NO: 168 - protein, 122 aa, synthetic construct
QCQLQESGGGLVQAGGSLRLSCTAFRSVGVIDVMGWYRQAPGKQRELVATVTSGSSTTYADSVKGRFAISRDNAKTVSLQMNSLKPEDTAVYYCADDDEGKLLNRRSVWYYWSQGTQVTVSS

SEQ ID NO: 169 - protein, 122 aa, synthetic construct
QVQLQESGGGLVQAGGSLRLSCTALRSVGSFNGMGWYRQSPGNQRELVATVTSDTSTTYRDSVKGRAFAISRDSAKTVSLQMNNLKPEDTAVYYCADEEGHHLNRRSTVWYWGQGTQVTVSS

SEQ ID NO: 170 - protein, 254 aa, synthetic construct
RCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSM

SEQ ID NO: 171 - protein, 242 aa, synthetic construct
DPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVH

SEQ ID NO: 172 - protein, 123 aa, synthetic construct
QVQLVESGGGSVQAGGSLRLSCAASGRTLRRYRMGWFRQALGKEREFVAGFTWSGGYAYADSVKGRFAISGDNAKNTGYLQMNSLKPEDTAVYYCALSRQVSLGPGPPNFDYWGQGTQVTVSS

SEQ ID NO: 173 - protein, 103 aa, synthetic construct
QVQLVQPGGSLRLFCVASEEFFSIYAMGWYRQAPGKQHEMVARFTRDGKITYADSAKGRFTITRDAKNTLNLQMNGLIPEDTAVYYCNINHYWGQGTQVTVSS

SEQ ID NO: 174 - protein, 120 aa, synthetic construct
DVQLVESGGGLVQPGGSLRLSCSVSGNIDRFYAMGWYRQAPGKQRELVAQLTNNEITTYGDSVEGQFSISGDFDANTVYLQMDSLKPEDTAVYYCHAHVTTTRWSQDYYWGQGTRVTVSS

SEQ ID NO: 175 - protein, 110 aa, synthetic construct
QVKLEESGGGSVQAGESLRLSCTASGITFRDYDDWYRQAPGKEREWLATITPSGTTHYPDSVKGRATISRDSAKNTVYLQMNSLKPEDTARYECNTLAYWGSGTQVTVSS

SEQ ID NO: 176 - protein, 121 aa, synthetic construct
QVQLQESGGGLVQPGGSLRLSCAASGSVFSIYAMAWYRQAPGKQRELVAVITSGGATNYADSVKGRFTISRDIAKKTLYLQMNTLKPEDTAVYYCYAHLLIQPFDRFYDYWGQGTQVTVSS

SEQ ID NO: 177 - protein, 128 aa, synthetic construct
EVQLVESGGGLVQPGGSLRLSCVASGFTFKDYGMTWVRQAPGKALEWVSDINSAGDGIYYSDSVKGRFTISRDDSKGTLYLQMNSLTPEDTAIYYCAAERQRAGDVKRSLAPITAHIWGQGTQVTVSS

SEQ ID NO: 178 - protein, 125 aa, synthetic construct
QVQLVESGGGLVQAGGSLRLSCAASGGAFSTYTMGWFRQAPGKEREFVAAIAWTGTHTYYSDSVKGRFTISRDNDKNTVFLQMNSLKPEDTAVYYCAQASSRYRAVTDSLSENHWGPGTQVTVST

SEQ ID NO: 179 - protein, 122 aa, synthetic construct
QVKLEESGGGLVRAGGSLRLSCAASGGTFSTYAMAWFRQAPGKEREFVAAITWGGGSTYYEDSVKGRFTISRDNAKNTGVLQMNNLDVDDTAVYYCYAFGRSRAGDANGDYWGQGTQVTVSS

SEQ ID NO: 180 - protein, 120 aa, synthetic construct
QVQLVETGGGLVRAGGSLRLSCAASGRTADIYNIGWFRQAPGKEREFVAAITWIGRTPYYADAVKGRFAFSTDSAKNTVSLQMDNLKPEDTGVYYCNAAHYLEGNTDYYWGQGTQVTVSS

SEQ ID NO: 181 - protein, 120 aa, synthetic construct; identical to SEQ ID NO: 180

SEQ ID NO: 182 - protein, 239 aa, synthetic construct
QVQLVESGGGLVQPGGSLRLSCAASGFTFSSYWMSWVRQAPGKGLEWVANINEDGSAKFYVDSVKGRFTISRDNAKNSLYLQMNSLRAEDTAVYFCARDLRSGRYWGQGTLVTVSSGGGGSGGGGSGGGGSEIVLTQSPGTLSLSPGGRATLSCRASQSISGSFLAWYQQKPGQAPRLLIYGASSRATGIPDRFSGSGSGTDFTLTISRLEPEDFAVYYCQQYGSSPPTFGLGTKLEIK

SEQ ID NO: 183 - protein, 239 aa, synthetic construct
QVNLRESGGGLVQPGGSLRLSCAASGFTFSSYEMNWVRQAPGKGLEWVSYISSSGSTIYYADSVKGRFTISRDNAKNSLYLQMNSLRAEDTAVYYCAREALGSSWEWGQGTTVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIK

SEQ ID NO: 184 - protein, 245 aa, synthetic construct
EVQLVESGGGVVQPGGSLRLSCAASGFTFDDYAMHWVRQAPGKGLEWVSLISGDGGSTYYADSVKGRFTISRDNSKNTLYLQMNSLRVEDTAVYYCARVFDSYYMDVWGKGTTVTVSSGGGGSGGGGSGSGGSEIVLTQSPLSLPVTPGQPASISCRSSQSLVYTDGNTYLNWFQQRPGQSPRRLIYKVSNRDSGVPDRFSGSGSDTDFTLKISRVEAEDVGIYYCMQGTHWSFTFGQGTRLEIK

SEQ ID NO: 185 - protein, 245 aa, synthetic construct
EVQLQQSGPGLVRPSETLSLTCTVSGGPVRSGSHYWNWIRQPPGRGLEWIGYIYYSGSTNYNPSLENRVTISIDTSNNHFSLKLSSVTAADTALYFCARGTATFDWNFPFDSWGQGTLVTVSSGGGGSGGGGSGSGGSDIQMTQSPSSLSASIGDRVTITCRASQSISSYLNWYQQKPGKAPKLLIYAASSLQSGVPSRFSGSGSGTDFTLTISSLQPEDFATYYCQQSYSTPWTFGQGTKLEIK

SEQ ID NO: 186 - protein, 246 aa, synthetic construct
QVQLVQSGAEVKEPGASVKVSCKAPANTFSDHVMHWVRQAPGQRFEWMGYIHAANGGTHYSQKFQDRVTITRDTSANTVYMDLSSLRSEDTAVYYCARGGYNSDAFDIWGQGTMVTVSSGGGGSGGGGSGGGGSGGGGSDIVMTQSPSSVSASVGDRVTITCRASQDISSWLAWYQQKPGKAPKLLIYAASSLQSGVPSRFNGSGSGTDFTLTISSLQPEDFATYYCQQSYSTPLTFGGGTKVEIK

SEQ ID NO: 187 - protein, 246 aa, synthetic construct
EVQLVQSGGGVVRSGRSLRLSCAASGFTFNSYGLHWVRQAPGKGLEWVALIEYDGSNKYYGDSVKGRFTISRDKSKSTLYLQMDNLRAEDTAVYYCAREGNEDLAFDIWGQGTLVTVSSGGGGSGGGGSGGGGSGGGGSEIVLTQSPSSLSASVGDRVTITCQASQFIKKNLNWYQHKPGKAPKLLIYDASSLQTGVPSRFSGNRSGTTFSFTISSLQPEDVATYYCQQHDNLPLTFGGGTKVEIK

SEQ ID NO: 188 - protein, 247 aa, synthetic construct
QVQLQESGPGLVKPSETLSLTCTVSGGSISSSSYYWGWIRQPPGKGLEWIGSIYYSGSTYYNPSLKSRVSISVDTSKNQFSLKLKYVTAADTAVYYCATPGTYYDFLSGYYPFYWGQGTLVTVSSGGGGSGGGGSGGGGSDIVMTQSPSSLSASVGDRVTITCRASQGISSYLAWYQQKPGKAPKLLIYAASTLQSGVPSRFSGSGSGTDFTLTISSLQPEDFATYYCQQLNSYPYTFGQGTKLEIK

SEQ ID NO: 189 - protein, 247 aa, synthetic construct
QVQLVQSGAEVKKSGASVKVSCKASGYPFTGYYIQWVRQAPGQGLEWMGWIDPNSGNTGYAQKFQGRVTMTRNTSISTAYMELSSLRSEDTAVYYCASDSYGYYYGMDVWGQGTLVTVSSGGGGSGGGGSGGGGSGGGGSDIQMTQSPSSLSASVGDRVTFTCRASQGISSALAWYQQKPGKPPKLLIYDASSLESGVPSRFSGSGSGTDFTLTISSLQPEDFATYYCQQFNNYPLTFGGGTKVEIK

SEQ ID NO: 190 - protein, 247 aa, synthetic construct
EVQLQQSGAEVKKPGSSVKVSCKASGGTFSSYAISWVRQAPGQGLEWMGGIIPIFGTANYAQKFQGRVTITADESTSTAYMELSSLRSEDTAVYYCARDLEMATIMGGYWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLDVVFGGGTKLTVL

SEQ ID NO: 191 - protein, 248 aa, synthetic construct
QVQLVQSGGGLVQPGGSLRLSCAASGFTFSSYSMNWVRQAPGKGLEWVSYISSSSSTIYYADSVKGRFTISRDNAKNSLYLQMNSLRAEDTAVYYCARDLSVRAIDAFDIWGQGTMVTVSSGGGGSGGGGSGGGGSGGGGSDIVLTQSPSSLSASVGDRVTITCQASQDISNYLNWYQQKPGKAPKLLIYDASNLETGVPSRFSGSGSGTDFTFTISSLQPEDFATYYCQQAYSTPFTFGPGTKVEIK

SEQ ID NO: 192 - protein, 249 aa, synthetic construct
QVQLQESGAGLLKPSETLSLTCAVYGGSFSGYYWSWIRQPPGKGLEWVGEINHSGSTNYNPSLKSRVTISVDTSKNQFSLKLSSVTAADTAVYYCARGSGLVVYAIRVGSGWFDYWGQGTLVTVSSGGGGSGGGDSGGGGSDIQMTQSPSSLSASVGDRVTITCRASQSISSYLNWYQQKPGKAPKLLMYAASSLQSGVPSRFSGSGSGTDFTLTISSLQPEDFATYYCQQSYSTPPWTFGQGTKVDIK

SEQ ID NO: 193 - protein, 255 aa, synthetic construct
QVQLVESGGGLVQPGGSLRLSCAASGFNVSSNYMTWVRQAPGKGLEWVSVIYSGGATYYGDSVKGRFTVSRDNSKNTVYLQMNRLTAEDTAVYYCARDRLYCGNNCYLYYYYGMDVWGQGTLVTVSSGGGGSGGGGSGGGGSGGGGSDIQVTQSPSSLSASVGDRVTITCRASQSISSYLNWYQQKPGKAPKLLIYAASSLQSGVPSRFSGSGSGTDFTLTISSLQPEDFATYYCQQSYSTPPLTFGQGTKVEIK

SEQ ID NO: 194 - protein, 255 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDPSSSGSYYMEDSYYYGMDVWGQGTTVTVSSGGGGSGGGGSGGGGSNFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSNQVVFGGGTKLTVL

SEQ ID NO: 195 - protein, 245 aa, synthetic construct
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCARDPYSSSWHDAFDIWGQGTMVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVDIK

SEQ ID NO: 196 - protein, 201 aa, synthetic construct [sequence omitted]

SEQ ID NO: 197 - protein, 250 aa, synthetic construct
DIQMTQSPSSLSASVGDRVTITCKSSQSLLYSDNQKNYLAWYQQKPGKAPKLLIYWASTRESGVPSRFSGSGSGTDFTLTISSLQPEDFATYYCQQYYTYPYTFGQGTKLEIKGGGGSGGGGSGGGGSEVQLVQSGAEVKKPGASVKVSCKASGYTFTGYHMHWVRQAPGQRLEWMGRINPYNGAASHNQKFKDRVTITRDTSASTAYMELSSLRSEDTAVYYCARGWDYDGGYYAMDYWGQGTLVTVSS

SEQ ID NO: 198 - protein, 245 aa, synthetic construct
ENVLTQSPAIMSASPGEKVTMTCRASSNVISSYVHWYQQRSGASPKLWIYSTSNLASGVPARFSGSGSGTSYSLTISSVEAEDAATYYCQQYSGYPLTFGAGTKLELGGGGSGGGGSGGGGSDIQLQESGPGLVKPSQSLSLTCSVTGYSITSAYYWNWIRQFPGNKLEWMGYISYDGRNNYNPSLKNRISITRDTSKNQFFLKLNSVTTEDTATYYCAKEGDYDVGNYYAMDYWGQGTSVTVSS

SEQ ID NO: 199 - protein, 250 aa, synthetic construct
DIVMSQSPSSLAVSVGEKVTMSCKSSQSLLYSDNQKNYLAWYQQKPGQSPKLLIYWASTRESGVPDRFTGSGSGTDFTLTISSVKAEDLAVYYCQQYYTYPYTFGGGTKLEIKGGGGSGGGGSGGGGSEVQLQQSGPELVKPGASVKISCKASGYSFTGYHMHWVKQSHVKSLEWIGRINPYNGAASHNQKFKDKATLTVDKSSSTAYMELHSLTSEDSAVYYCARGWDYDGGYYAMDYWGQGTSVTVSS

SEQ ID NO: 200 - protein, 242 aa, synthetic construct
QVQLQQSGAELMKPGASVKISCKATGYTFSSYWIEWVKQRPGHGLEWIGEIFPGSGSIKYNEKFKGKATFTADTSSNTAYMQLSSLTSEDSAVHYCARGGTYNDYSLFDYWGQGTTLTVSSGGGGSGGGGSGGGGSQIVLTQSPAIMSASPGEKVTMTCSASSSVSYMYWYQQKPGSSPRLLIFDTSNLASGVPVRFSGSGSGTSYSLTISRMEAEDAATYYCQQWSSYPLTFGAGTKLELK

SEQ ID NO: 201 - protein, 238 aa, synthetic construct
QVQLQQPGSDLVRPGASVKLSCKASGYTFTRYWMHWVKQRPGHGLEWIGYIYPGSGTSNYDEKFKSKATLTVDTSSSTAYMQLSSLTSEDSAVYYCTREARYTMDYWGQGTSVTVSSGGGGSGGGGSGGGGSQIVLTQSPAIMSASPGEKVTMTCSASSSVSYIYWYQQKPGSSPGLLIYDTSNLASGVPVRFSGSGSGTSYSLTISRMEAEDAATYYCQQWSSFPPTFGAGTKLELK

SEQ ID NO: 202 - protein, 289 aa, synthetic construct
MLLLVTSLLLCELPHPAFLLIPSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSGILEQQG

SEQ ID NO: 203 - protein, 241 aa, synthetic construct
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 204 - protein, 241 aa, synthetic construct
QVQLQQSGAEVKKPGASVKVSCKASGYTFTGYYMHWVRQAPGQGLEWMGWINPNSGGTNYAQNFQGRVTMTRDTSISTAYMELRRLRSDDTAVYYCASGWDFDYWGQGTLVTVSSGGGGSGGGGSGGGGSGGGGSDIRMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 205 - protein, 242 aa, synthetic construct
QVQLVESGGGLVQPGGSLRLSCAASGFTFSSYWMHWVRQVPGKGLVWVSRINTDGSTTTYADSVEGRFTISRDNAKNTLYLQMNSLRDDDTAVYYCVGGHWAVWGQGTTVTVSSGGGGSGGGGSGGGGSGGGGSDIQMTQSPSTLSASVGDRVTITCRASQSISDRLAWYQQKPGKAPKLLIYKASSLESGVPSRFSGSGSGTEFTLTISSLQPDDFAVYYCQQYGHLPMYTFGQGTKVEIK

SEQ ID NO: 206 - protein, 244 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 207 - protein, 244 aa, synthetic construct
QVQLVQSGGGLVQPGRSLRLSCAASGFTFDDYAMHWVRQAPGKGLEWVSGISWNSGSIGYADSVKGRFTISRDNAKNSLYLQMNSLRAEDTAVYYCAKDGSSSWSWGYFDYWGQGTLVTVSSGGGGSGGGGSGGGGSSSELTQDPAVSVALGQTVRTTCQGDALRSYYASWYQQKPGQAPMLVIYGKNNRPSGIPDRFSGSDSGDTASLTITGAQAEDEADYYCNSRDSSGYPVFGTGTKVTVL

SEQ ID NO: 208 - protein, 246 aa, synthetic construct
QVQLVQSGAEVKKPGAPVKVSCKASGYTFTGYYMHWVRQAPGQGLEWMGWINPNSGGTNYAQKFQGRVTMTRDTSISTAYMELSRLRSDDTAVYYCARGEWDGSYYYDYWGQGTLVTVSSGGGGSGGGGSGGGGSGGGGSDIVLTQTPSSLSASVGDRVTITCRASQSINTYLNWYQHKPGKAPKLLIYAASSLQSGVPSRFSGSGSGTDFTLTISSLQPEDFATYYCQQSFSPLTFGGGTKLEIK

SEQ ID NO: 209 - protein, 246 aa, synthetic construct
QVQLQQSGAEVKKPGASVKVSCKTSGYPFTGYSLHWVRQAPGQGLEWMGWINPNSGGTNYAQKFQGRVTMTRDTSISTAYMELSRLRSDDTAVYYCARDHYGGNSLFYWGQGTLVTVSSGGGGSGGGGSGGGGSGGGGSDIQLTQSPSSISASVGDTVSITCRASQDSGTWLAWYQQKPGKAPNLLMYDASTLEDGVPSRFSGSASGTEFTLTVNRLQPEDSATYYCQQYNSYPLTFGGGTKVDIK

SEQ ID NO: 210 - protein, 246 aa, synthetic construct
QVQLVQSGAEVKKPGASVKVSCKASGYTFTGYYMHWVRQAPGQGLEWMGRINPNSGGTNYAQKFQGRVTMTTDTSTSTAYMELRSLRSDDTAVYYCARTTTSYAFDIWGQGTMVTVSSGGGGSGGGGSGGGGSGGGGSDIQLTQSPSTLSASVGDRVTITCRASQSISTWLAWYQQKPGKAPNLLIYKASTLESGVPSRFSGSGSGTEFTLTISSLQPDDFATYYCQQYNTYSPYTFGQGTKLEIK

SEQ ID NO: 211 - protein, 246 aa, synthetic construct
EVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQEPAVSVALGQTVRITCQGDSLRSYYASWYQQKPGQAPVLVIFGRSRRPSGIPDRFSGSSSGNTASLIITGAQAEDEADYYCNSRDNTANHYVFGTGTKLTVL

SEQ ID NO: 212 - protein, 246 aa, synthetic construct
EVQLVESGGGLVQPGRSLRLSCAASGFTFDDYAMHWVRQAPGKGLEWVSGISWNSGSTGYADSVKGRFTISRDNAKNSLYLQMNSLRAEDTALYYCAKDSSSWYGGGSAFDIWGQGTMVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRGSSGNHYVFGTGTKVTVL

SEQ ID NO: 213 - protein, 246 aa, synthetic construct
QITLKESGPALVKPTQTLTLTCTFSGFSLSTAGVHVGWIRQPPGKALEWLALISWADDKRYRPSLRSRLDITRVTSKDQVVLSMTNMQPEDTATYYCALQGFDGYEANWGPGTLVTVSSGGGGSGGGGSGGGGSGGGGSDIVMTQSPSSLSASAGDRVTITCRASRGISSALAWYQQKPGKPPKLLIYDASSLESGVPSRFSGSGSGTDFTLTIDSLEPEDFATYYCQQSYSTPWTFGQGTKVDIK

SEQ ID NO: 214 - protein, 247 aa, synthetic construct
QVQLVQSWAEVKKPGASVKVSCKASGYTFTSYYMHWVRQAPGQGLEWMGIINPSGGSTSYAQKFQGRVTMTRDTSTSTVYMELSNLRSEDTAVYYCARSPRVTTGYFDYWGQGTLVTVSSGGGGSGGGGSGGGGSGGGGSDIQLTQSPSTLSASVGDRVTITCRASQSISSWLAWYQQKPGKAPKLLIYKASSLESGVPSRFSGSGSGTEFTLTISSLQPDDFATYYCQQYSSYPLTFGGGTRLEIK

SEQ ID NO: 215 - protein, 248 aa, synthetic construct
QVQLVQSGAEVKKPGASVEVSCKASGYTFTSYYMHWVRQAPGQGLEWMGIINPSGGSTGYAQKFQGRVTMTRDTSTSTVHMELSSLRSEDTAVYYCARGGYSSSSDAFDIWGQGTMVTVSSGGGGSGGGGSGGGGSGGGGSDIQMTQSPPSLSASVGDRVTITCRASQDISSALAWYQQKPGTPPKLLIYDASSLESGVPSRFSGSGSGTDFTLTISSLQPEDFATYYCQQFSSYPLTFGGGTRLEIK

SEQ ID NO: 216 - protein, 249 aa, synthetic construct
QVQLVQSGGGLVKPGGSLRLSCEASGFIFSDYYMGWIRQAPGKGLEWVSYIGRSGSSMYYADSVKGRFTFSRDNAKNSLYLQMNSLRAEDTAVYYCAASPVVAATEDFQHWGQGTLVTVSSGGGGSGGGGSGGGGSGGGGSDIVMTQTPATLSLSPGERATLSCRASQSVTSNYLAWYQQKPGQAPRLLLFGASTRATGIPDRFSGSGSGTDFTLTINRLEPEDFAMYYCQQYGSAPVTFGQGTKLEIK

SEQ ID NO: 217 - protein, 249 aa, synthetic construct
QVQLVQSGAEVRAPGASVKISCKASGFTFRGYYIHWVRQAPGQGLEWMGIINPSGGSRAYAQKFQGRVTMTRDTSTSTVYMELSSLRSDDTAMYYCARTASCGGDCYYLDYWGQGTLVTVSSGGGGSGGGGSGGGGSGGGGSDIQMTQSPPTLSASVGDRVTITCRASENVNIWLAWYQQKPGKAPKLLIYKSSSLASGVPSRFSGSGSGAEFTLTISSLQPDDFATYYCQQYQSYPLTFGGGTKVDIK

SEQ ID NO: 218 - protein, 249 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKREAAAGHDWYFDLWGRGTLVTVSSGGGGSGGGGSGGGGSGGGGSDIRVTQSPSSLSASVGDRVTITCRASQSISSYLNWYQQKPGKAPKLLIYAASSLQSGVPSRFSGSGSGTDFTLTISSLQPEDFATYYCQQSYSIPLTFGQGTKVEIK

SEQ ID NO: 219 - protein, 249 aa, synthetic construct
QVQLQQSGAEVKKPGASVKVSCKASGYTFTNYYMHWVRQAPGQGLEWMGIINPSGGYTTYAQKFQGRLTMTRDTSTSTVYMELSSLRSEDTAVYYCARIRSCGGDCYYFDNWGQGTLVTVSSGGGGSGGGGSGGGGSGGGGSDIQLTQSPSTLSASVGDRVTITCRASENVNIWLAWYQQKPGKAPKLLIYKSSSLASGVPSRFSGSGSGAEFTLTISSLQPDDFATYYCQQYQSYPLTFGGGTKVDIK

SEQ ID NO: 220 - protein, 250 aa, synthetic construct
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARWKVSSSSPAFDYWGQGTLVTVSSGGGGSGGGGSGGGGSGGGGSEIVLTQSPATLSLSPGERAILSCRASQSVYTKYLGWYQQKPGQAPRLLIYDASTRATGIPDRFSGSGSGTDFTLTINRLEPEDFAVYYCQHYGGSPLITFGQGTRLEIK

SEQ ID NO: 221 - protein, 250 aa, synthetic construct
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKGYSRYYYYGMDVWGQGTTVTVSSGGGGSGGGGSGGGGSGGGGSEIVMTQSPATLSLSPGERAILSCRASQSVYTKYLGWYQQKPGQAPRLLIYDASTRATGIPDRFSGSGSGTDFTLTINRLEPEDFAVYYCQHYGGSPLITFGQGTKVDIK

SEQ ID NO: 222 - protein, 251 aa, synthetic construct
QVQLVQSGGGLVQPGGSLRLSCAASGFTFSSYWMHWVRQAPGKGLVWVSRINSDGSSTSYADSVKGRFTISRDNAKNTLYLQMNSLRAEDTAVYYCVRTGWVGSYYYYMDVWGKGTTVTVSSGGGGSGGGGSGGGGSGGGGSEIVLTQSPGTLSLSPGERATLSCRASQSVSSNYLAWYQQKPGQPPRLLIYDVSTRATGIPARFSGGGSGTDFTLTISSLEPEDFAVYYCQQRSNWPPWTFGQGTKVEIK

SEQ ID NO: 223 - protein, 253 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 224 - protein, 253 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARYRLIAVAGDYYYYGMDVWGQGTMVTVSSGGGGSGGGGSGGGGSGGGGSDIQMTQSPSSVSASVGDRVTITCRASQGVGRWLAWYQQKPGTAPKLLIYAASTLQSGVPSRFSGSGSGTDFTLTINNLQPEDFATYYCQQANSFPLTFGGGTRLEIK

SEQ ID NO: 225 - protein, 253 aa, synthetic construct
QVQLVQSGAEVRRPGASVKISCRASGDTSTRHYIHWLRQAPGQGPEWMGVINPTTGPATGSPAYAQMLQGRVTMTRDTSTRTVYMELRSLRFEDTAVYYCARSVVGRSAPYYFDYWGQGTLVTVSSGGGGSGGGGSGGGGSGGGGSDIQMTQSPSSLSASVGDRVTITCRASQGISDYSAWYQQKPGKAPKLLIYAASTLQSGVPSRFSGSGSGTDFTLTISYLQSEDFATYYCQQYYSYPLTFGGGTKVDIK

SEQ ID NO: 226 - protein, 255 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVAGGIYYYYGMDVWGQGTTITVSSGGGGSGGGGSGGGGSGGGGSDIVMTQTPDSLAVSLGERATISCKSSHSVLYNRNNKNYLAWYQQKPGQPPKLLFYWASTRKSGVPDRFSGSGSGTDFTLTISSLQPEDFATYFCQQTQTFPLTFGQGTRLEIN

SEQ ID NO: 227 - protein, 248 aa, synthetic construct
EVQLVESGGGLVQPGGSLRLSCAASGFTFSDYGMHWVRQAPGKGLEWVSSIYGSGGHTGYADSVKGRFTISRDNSKNTLYLQMNSLRAEDTAVYYCAKQHAYRYSYAFDVWGQGTLVTVSSGGGGSGGGGSGGGGSGGGGSDIQMTQSPSSLSASVGDRVTITCRASQSISNWLNWYQQKPGKAPKLLIYATSSLQSGVPSRFSGSGSGTDFTLTISSLQPEDFATYYCQQSYSFPFTFGQGTKVEIK

SEQ ID NO: 228 - protein, 248 aa, synthetic construct
DIQMTQSPSSLSASVGDRVTITCRASQSISNWLNWYQQKPGKAPKLLIYATSSLQSGVPSRFSGSGSGTDFTLTISSLQPEDFATYYCQQSYSFPFTFGQGTKVEIKGGGGSGGGGSGGGGSGGGGSEVQLVESGGGLVQPGGSLRLSCAASGFTFSDYGMHWVRQAPGKGLEWVSSIYGSGGHTGYADSVKGRFTISRDNSKNTLYLQMNSLRAEDTAVYYCAKQHAYRYSYAFDVWGQGTLVTVSS

SEQ ID NO: 229 - protein, 248 aa, synthetic construct
EVQLVESGGGLVQPGGSLRLSCAASGFTFSSYAMHWVRQAPGKGLEWVSGISGSGGTTYYADSVKGRFTISRDNSKNTLYLQMNSLRAEDTAVYYCAKEVEGQSQEYFDIWGQGTLVTVSSGGGGSGGGGSGGGGSGGGGSDIQMTQSPSSLSASVGDRVTITCRASQSIANYLNWYQQKPGKAPKLLIYAASNLQSGVPSRFSGSGSGTDFTLTISSLQPEDFATYYCQQSYSFPYTFGQGTKVEIK

SEQ ID NO: 230 - protein, 248 aa, synthetic construct
DIQMTQSPSSLSASVGDRVTITCRASQSIANYLNWYQQKPGKAPKLLIYAASNLQSGVPSRFSGSGSGTDFTLTISSLQPEDFATYYCQQSYSFPYTFGQGTKVEIKGGGGSGGGGSGGGGSGGGGSEVQLVESGGGLVQPGGSLRLSCAASGFTFSSYAMHWVRQAPGKGLEWVSGISGSGGTTYYADSVKGRFTISRDNSKNTLYLQMNSLRAEDTAVYYCAKEVEGQSQEYFDIWGQGTLVTVSS

SEQ ID NO: 231 - protein, 248 aa, synthetic construct
EVQLVESGGGLVQPGGSLRLSCAASGFTFSSYAMSWVRQAPGKGLEWVSGISGSGGSTGYADSVKGRFTISRDNSKNTLYLQMNSLRAEDTAVYYCAKHGQVGGISVFDIWGQGTLVTVSSGGGGSGGGGSGGGGSGGGGSDIQMTQSPSSLSASVGDRVTITCRASQSISNWLNWYQQKPGKAPKLLIYATSRLQSGVPSRFSGSGSGTDFTLTISSLQPEDFATYYCQQSYSFPWTFGQGTKVEIK

SEQ ID NO: 232 - protein, 248 aa, synthetic construct
DIQMTQSPSSLSASVGDRVTITCRASQSISNWLNWYQQKPGKAPKLLIYATSRLQSGVPSRFSGSGSGTDFTLTISSLQPEDFATYYCQQSYSFPWTFGQGTKVEIKGGGGSGGGGSGGGGSGGGGSEVQLVESGGGLVQPGGSLRLSCAASGFTFSSYAMSWVRQAPGKGLEWVSGISGSGGSTGYADSVKGRFTISRDNSKNTLYLQMNSLRAEDTAVYYCAKHGQVGGISVFDIWGQGTLVTVSS

SEQ ID NO: 233 - protein, 459 aa, synthetic construct
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAG

SEQ ID NO: 234 - protein, 459 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAGGGGGSGGGGSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 241 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 236 - protein, 234 aa, synthetic construct
AEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKR

SEQ ID NO: 237 - protein, 248 aa, synthetic construct
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSSGGGGSGGGGSGGGGSDVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 238 - protein, 259 aa, synthetic construct
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 239 - protein, 245 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 240 - protein, 272 aa, synthetic construct
MASFPLLLTLLTHCAGSWASYELTQPPSVSVSPGQTARITCSGNALPKQYAYWYQQKPGQAPVLMIYKDSERPSGIPERFSGSSSGTTVTLTISGVQAEDEADYYCQSADSSHTYKVFGGGTKLTVLGGGGSGGGGSGGGGSGGGGSEVQLVQSGGGLVQPDRSLRLSCAASGFTFDDYAMHWVRQAPGKGLEWVSGISWNSGSIGYADSVKGRFTISRDNAKNSLYLQMNSLRAEDTALYYCAKDLGSYYGSGSGMDVWGQGTTVTVSSGG

SEQ ID NO: 241 - protein, 270 aa, synthetic construct
MASFPLLLTLLTHCAGSWASYELTQPPSVSVSPGQTARITCSGDALPKHYASWYQQKPGQAPVLLIYKDTERPSGIPERFSGSSSGTTVTLTISGVQAEDEADYYCQSADSSVTVVFGGGTKLTVLGGGGSGGGGSGGGGSGGGGSQVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKDIGSSGWYGAFDIWGQGTTVTVSSGG

SEQ ID NO: 242 - protein, 269 aa, synthetic construct
MASFPLLLTLLTHCAGSWASSELTQDPAVSVALGQTVRITCQGDSLRSYYASWYQQKPGQAPVLVIYGKNNRPSGIPDRFSGSSSGNTASLTITGAQAEDEADYYCQAWDSSTAVVFGGGTKLTVLGGGGSGGGGSGGGGSGGGGSQVQLVQSGGGLVQPGRSLRLSCAASGFTFDDYAMHWVRQAPGKGLEWVSGISWNSGIIGYADSVKGRFTISRDNAKNSLYLQMNSLRAEDTALYYCAKDLSSSWYGAFDIWGQGTMVTVSSGG

SEQ ID NO: 243 - protein, 267 aa, synthetic construct
MASFPLLLTLLTHCAGSWASYELTQPPSVSVSPGQTARITCSGDALPKQYAYWYQQKPGQAPVLVIYKDSERPSGIPERFSGSSSGTTVTLTISGVQAEDEADYYCQSADSSGTWFGGGTKLTVLGGGGSGGGGSGGGGSGGGGSEVQLVQSGGGLVQPGRSLRLSCAASGFTFDDYAMHWVRQAPGKGLEWVSGISWNSGSIVYADSVKGRFTISRDNAKNSLYLQMNSLRAEDTALYYCAKDRSSWYGGEDYWGQGTLVTVSSGG

SEQ ID NO: 244 - protein, 273 aa, synthetic construct
MASFPLLLTLLTHCAGSWASYELMQPPSVSVSPGQTARITCSGDALPKQYAYWYQQKPGQAPVLVIYKDSERPSGIPERFSGSSSGTTVTLTISGVQAEDEADYYCQSADSSGTYRDVVFGGGTKLTVLGGGGSGGGGSGGGGSGGGGSEVQLVQSGGGLVQPGRSLRLSCAASGFTFDDYAMHWVRQAPGKGLEWVSGISWNSGSIGYADSVKGRFTISRDNAKNSLYLQMNSLRAEDTALYYCAKGLGSSGYYDAFDIWGQGTTVTVSSGG

SEQ ID NO: 245 - protein, 271 aa, synthetic construct
MASFPLLLTLLTHCAGSWAQAGLTQPPSVSVSPGQTARITCSGDALPKQFTYWYQQKPGQAPVLVIYQDRERPSGIPERFSGSSSGTKVTLTISGVQAEDEADYYCQSADSSGTYRVFGGGTKLTVLGGGGSGGGGSGGGGSGGGGSEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDAGSSGYWSYEDYWGQGTLVTVSSGG

SEQ ID NO: 246 - protein, 122 aa, synthetic construct
EVQLVESGGDSVQTGGSLRLSCLASGYIGSGYCLAWFRQAPGKERKKVANIVIGVGRPYYADSVKGRFTISQDNAKNTLYLQMNSLKPEDTAMYYCAASKFGSWSCGDYNYWGQGTQVTVSS

SEQ ID NO: 247 - protein, 249 aa, synthetic construct
DIVLTQSPDSLAVSLGERATINCRASESVDNYGNTFMHWYQQKPGQPPKLLIYRASNLESGVPDRFSGSGSRTDFTLTISSLQAEDVAVYYCQQSNEDPPTFGQGTKLEIKGGGGSGGGGSGGGGSGGGGSQIQLVQSGSELKKPGASVKVSCKASGYIFTNYGMNWVRQAPGQGLEWMGWINTYTGESTYSADFKGRFVFSLDTSVSTAYLQINALKAEDTAVYYCARSGGYDPMDYWGQGTTVTVSS

SEQ ID NO: 248 - protein, 249 aa, synthetic construct
DIVLTQSPDSLAVSLGERATINCRASESVDNYGNTFMHWYQQKPGQPPKLLIYRASNLESGVPDRFSGSGSRTDFTLTISSLQAEDVAVYYCQQSNEDPPTFGQGTKLEIKGGGGSGGGGSGGGGSGGGGSQIQLVQSGAEVKKPGASVKVSCKASGYIFTNYGMNWVRQAPGQRLEWMGWINTYTGESTYSADFKGRVTITLDTSASTAYMELSSLRSEDTAVYYCARSGGYDPMDYWGQGTTVTVSS

SEQ ID NO: 249 - protein, 249 aa, synthetic construct
EIVLTQSPATLSLSPGERATLSCRASESVDNYGNTFMHWYQQKPGQAPRLLIYRASNLESGIPARFSGSGSRTDFTLTISSLEPEDVAVYYCQQSNEDPPTFGQGTKLEIKGGGGSGGGGSGGGGSGGGGSQIQLVQSGSELKKPGASVKVSCKASGYIFTNYGMNWVRQAPGQGLEWMGWINTYTGESTYSADFKGRFVFSLDTSVSTAYLQINALKAEDTAVYYCARSGGYDPMDYWGQGTTVTVSS

SEQ ID NO: 250 - protein, 249 aa, synthetic construct
EIVLTQSPATLSLSPGERATLSCRASESVDNYGNTFMHWYQQKPGQAPRLLIYRASNLESGIPARFSGSGSRTDFTLTISSLEPEDVAVYYCQQSNEDPPTFGQGTKLEIKGGGGSGGGGSGGGGSGGGGSQIQLVQSGAEVKKPGASVKVSCKASGYIFTNYGMNWVRQAPGQRLEWMGWINTYTGESTYSADFKGRVTITLDTSASTAYMELSSLRSEDTAVYYCARSGGYDPMDYWGQGTTVTVSS

SEQ ID NO: 251 - protein, 249 aa, synthetic construct
QIQLVQSGSELKKPGASVKVSCKASGYIFTNYGMNWVRQAPGQGLEWMGWINTYTGESTYSADFKGRFVFSLDTSVSTAYLQINALKAEDTAVYYCARSGGYDPMDYWGQGTTVTVSSGGGGSGGGGSGGGGSGGGGSDIVLTQSPDSLAVSLGERATINCRASESVDNYGNTFMHWYQQKPGQPPKLLIYRASNLESGVPDRFSGSGSRTDFTLTISSLQAEDVAVYYCQQSNEDPPTFGQGTKLEIK

SEQ ID NO: 252 - protein, 249 aa, synthetic construct
QIQLVQSGSELKKPGASVKVSCKASGYIFTNYGMNWVRQAPGQGLEWMGWINTYTGESTYSADFKGRFVFSLDTSVSTAYLQINALKAEDTAVYYCARSGGYDPMDYWGQGTTVTVSSGGGGSGGGGSGGGGSGGGGSEIVLTQSPATLSLSPGERATLSCRASESVDNYGNTFMHWYQQKPGQAPRLLIYRASNLESGIPARFSGSGSRTDFTLTISSLEPEDVAVYYCQQSNEDPPTFGQGTKLEIK

SEQ ID NO: 253 - protein, 249 aa, synthetic construct
QIQLVQSGAEVKKPGASVKVSCKASGYIFTNYGMNWVRQAPGQRLEWMGWINTYTGESTYSADFKGRVTITLDTSASTAYMELSSLRSEDTAVYYCARSGGYDPMDYWGQGTTVTVSSGGGGSGGGGSGGGGSGGGGSDIVLTQSPDSLAVSLGERATINCRASESVDNYGNTFMHWYQQKPGQPPKLLIYRASNLESGVPDRFSGSGSRTDFTLTISSLQAEDVAVYYCQQSNEDPPTFGQGTKLEIK

SEQ ID NO: 254 - protein, 249 aa, synthetic construct
QIQLVQSGAEVKKPGASVKVSCKASGYIFTNYGMNWVRQAPGQRLEWMGWINTYTGESTYSADFKGRVTITLDTSASTAYMELSSLRSEDTAVYYCARSGGYDPMDYWGQGTTVTVSSGGGGSGGGGSGGGGSGGGGSEIVLTQSPATLSLSPGERATLSCRASESVDNYGNTFMHWYQQKPGQAPRLLIYRASNLESGIPARFSGSGSRTDFTLTISSLEPEDVAVYYCQQSNEDPPTFGQGTKLEIK

SEQ ID NO: 255 - protein, 287 aa, synthetic construct
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWR

SEQ ID NO: 256 - protein, 243 aa, synthetic construct
EVQLQQSGAELVRPGASVRLSCTTSGFNIKDSFIHWVKQRTEQGLEWIGRIDPEDDETKYAPKFQGKATITADTSSNTAYLQLSSLTSEDTAVYYCASPIYGSREAWFAYWGQGTLVTVSAGGGGSGGGGSGGGGSDIQMTQSPASLSASLGETVTIECLASEDIYSNLAWYQQKPGKSPQLLIYDASSLQDGVPSRFSGSESGTQYSLEINSLQSEDAATYFCQQHHDYPLTFGSGTKLEIK

SEQ ID NO: 257 - protein, 243 aa, synthetic construct
EVQLQQSGAELVRPGASVRLSCTTSGFNIKDSFIHWVKQRTEQGLEWIGRIDPEDDETKYAPKFQGKATITADTSSNTAYLQLSSLTSEDTAVYYCASPIYGSREAWFAYWGQGTLVTVSAGGGGSGGGGSGGGGSDIQMTQSPASLSASLGETVTIECRASEDIYNGLAWYQQKPGKSPQLLIYNANSLHTGVPSRFSGSGSGTQYSLKINSLQSEDVASYFCQQYYNYPYTFGAGTKLELK

SEQ ID NO: 258 - protein, 237 aa, synthetic construct
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKGGGGSGGGGSGGGGSQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 259 - protein, 237 aa, synthetic construct
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 260 - protein, 246 aa, synthetic construct
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 261 - protein, 246 aa, synthetic construct
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSS

SEQ ID NO: 262 - protein, 246 aa, synthetic construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIK

SEQ ID NO: 263 - protein, 246 aa, synthetic construct
DVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 264 - protein, 246 aa, synthetic construct
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIK

SEQ ID NO: 265 - protein, 246 aa, synthetic construct
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 266 - protein, 246 aa, synthetic construct
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 267 - protein, 246 aa, synthetic construct
DVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIKGGGGSGGGGSGGGGSGGGGSEIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSS

SEQ ID NO: 268 - protein, 124 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 269 - protein, 127 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 270 - protein, 129 aa, synthetic construct
EVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSS

SEQ ID NO: 271 - protein, 241 aa, synthetic construct
EVQLLESGGGLVQPGGSLRLSCAASGFTFSSYAMSWVRQAPGKGLEWVSQIDPWGQETLYADSVKGRFTISRDNSKNTLYLQMNSLRAEDTAVYYCAKLTGRFDYWGQGTLVTVSSGGGGSGGGGSGGGGSTDIQMTQSPSSLSASVGDRVTITCRASQSISSYLNWYQQKPGKAPKLLIYSASQLQSGVPSRFSGSGSGTDFTLTISSLQPEDFATYYCQQGPGTPNTFGQGTKVEIKRA

SEQ ID NO: 272 - protein, 246 aa, synthetic construct
DIQMTQSPSTLSASVGDRVTITCRASQNINKWLAWYQQRPGKAPQLLIYKASSLESGVPSRFSGSGSGTEYTLTISSLQPDDFATYYCQQYNSYATFGQGTKVEIKRSRGGGGSGGGGSGGGGSLEMAQVQLVQSGAEVKKPGESLKISCKGSGYNFSNKWIGWVRQLPGRGLEWIAIIYPGYSDITYSPSFQGRVTISADTSINTAYLHWHSLKASDTAMYYCVRHTALAGFDYWGLGTLVTVSS

SEQ ID NO: 273 - protein, 249 aa, synthetic construct
QSVVTQPPSVSVAPGKTARITCGRNNIGSKSVHWYQQKPGQAPVLVVYDDSDRPSGIPERFSGSNSGNTATLTISRVEAGDEADYYCQVWDSSSDHVVFGGGTKLTVLGSRGGGGSGGGGSGGSLEMAEVQLVQSGGGVVRPGGSLRLSCAASGFTFDDYGMSWVRQAPGKGLEWVSGINWNGGSTGYADSVRGRFTISRDNAKNSLYLQMNSLRAEDTALYYCARERGYGYHDPHDYWGQGTLVTVSS

SEQ ID NO: 274 - protein, 250 aa, synthetic construct
QTVVTQPPSASGTPGQRVTISCSGSSSNIGSNYVYWYQQLPGTAPKLLIYRSNQRPSGVPDRFSGSKSGTSASLAISGPRSVDEADYYCAAWDDSLNGVVFGGGTKLTVLGSRGGGGSGGGGSGGGSLEMAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIPPYYGMDVWGQGTTVTVSS

SEQ ID NO: 275 - protein, 250 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIKRSRGGGGSGGGGSGGGGSLEMAQVQLQQSGPGLVKPSQTLSLTCAISGDSVSSNSAAWNWIRQSPSRGLEWLGRTYYGSKWYNDYAVSVKSRITINPDTSKNQFSLQLNSVTPEDTAVYYCARGRLGDAFDIWGQGTMVTVSS

SEQ ID NO: 276 - protein, 251 aa, synthetic construct
QSVLTQPPSVSGAPGQRVTISCTGSSSNIGAGYDVHWYQQLPGTAPKLLIYGNSNRPSGVPDRFSGSKSGTSASLAISGLQSEDEADYYCAAWDDSLNGYVFGTGTKLTVLGSRGGGGSGGGGSGGGGSLEMAEVQLVETGGGLLQPGGSLRLSCAASGFSVSGTYMGWVRQAPGKGLEWVALLYSGGGTYHPASLQGRFIVSRDSSKNMVYLQMNSLKAEDTAVYYCAKGGAGGGHFDSWGQGTLVTVSS

SEQ ID NO: 277 - protein, 253 aa, synthetic construct
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLGSRGGGGSGGGGSGGGGSLEMAQMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

(19) United States
(12) Patent Application Publication
(10) Pub. No.: US 2024/0285758 A1
(43) Pub. Date: Aug. 29, 2024

(54) SYNTHETIC RECEPTOR FOR CONDITIONAL ACTIVATION OF IMMUNE CELLS

(71) Applicant: Serotiny, Inc., San Francisco, CA (US)

(72) Inventors: Martin Jacko, Redwood City, CA (US); Aaron Goebel, San Francisco, CA (US); Justin Farlow, San Francisco, CA (US); Diego A. Vargas-Inchaustegui, San Francisco, CA (US); Yana Gofman, San Francisco, CA (US)

(21) Appl. No.: 18/534,111

(22) Filed: Dec. 8, 2023

Related U.S. Application Data
(63) Continuation of application No. PCT/US2022/032865, filed on Jun. 9, 2022.
(60) Provisional application No. 63/208,580, filed on Jun. 9, 2021.

(51) Int. Cl. A61K 39/00; C07K 14/705; C12N 5/0783

(52) U.S. Cl. CPC A61K 39/4631; A61K 39/4611; A61K 39/4613; A61K 39/464402; A61K 39/464403; A61K 39/464404; A61K 39/464412; A61K 39/464413; A61K 39/464416; A61K 39/464424; A61K 39/464468; A61K 39/46447; A61K 39/464471; A61K 39/464474; C07K 14/70503; C12N 5/0636; C12N 5/0646; C07K 2319/03; C07K 2319/30; C12N 2510/00

(57) ABSTRACT

Provided is a method of activating a recombinant immune cell expressing a synthetic receptor comprising an intracellular domain derived from killer cell immunoglobulin-like receptor 4 (KIR2DL4). Synthetic receptors described herein allow for the activation of recombinant immune cells against an antigen of interest without the deleterious effects of immune cell hyperactivation by existing CARs. The recombinant immune cells can thus be used to in the treatment of cancers or infectious diseases in subjects in need thereof, while limiting the hyperactivation of an immune response associated with traditional recombinant cell-based therapies.

### CROSS-REFERENCE TO RELATED APPLICATIONS

This application is a continuation of International (PCT) Patent Application No. PCT/US2022/032865, filed Jun. 9, 2022, which claims the benefit of and priority to U.S. Provisional Patent Application No. 63/208,580, filed Jun. 9, 2021, the disclosures of each which are hereby incorporated by reference in their entireties for all purposes.

### SEQUENCE LISTING XML

The instant application contains a Sequence Listing XML which has been submitted electronically in XML format and is hereby incorporated by reference in its entirety. Said XML copy, created on Mar. 14, 2024, is named SRT-001WOC1_SL.xml and is 298,147 bytes in size.

### BACKGROUND OF THE INVENTION

Synthetic immune receptors and recombinant cells expressing them are exciting technologies for personalized therapies for conditions such as cancer and infectious diseases. So-called “adoptive cell therapies” involve the introduction of a synthetic receptor (often a “chimeric antigen receptor” or “CAR”), that targets a selected antigen, into immune cells to generate recombinant immune cells (often referred to as “CAR-T” or “CAR-NK” cells depending on the type of immune cell chosen). Following infusion into a subject, the recombinant immune cells selectively target diseased cells or pathogens.

Although CAR-based therapies have shown promise, particularly in the treatment of B-cell malignancies, such therapies are not without risks. A particular limitation is toxicity associated with hyperactivation of an immune response (e.g., cytokine release syndrome, neurotoxicity, and anaphylaxis). As such, new technologies are needed to reduce the toxicity associated with CAR-based therapies. One approach to limiting the toxicity associated with CARs is the modulation of the intracellular signaling dynamics associated with CAR activation. Traditional CAR molecules comprise a CD3-zeta (CD3z) intracellular signaling domain, which contains immunoreceptor tyrosine-based activation motifs (ITAMs), that activates immune cells, such as T cells or NK cells, upon binding of an antigen to the CAR. However, robust activation of CD3z signaling can lead to hyperactivation of an immune response and resultant toxicity. Hyperactivation of immune cells may also lead to immune exhaustion, wherein immune cells lose activity to targets as the immune response persists.

One alternative approach is to replace the CD3z intracellular signaling domain with an alternative domain with reduced toxicity upon activation. The killer cell immunoglobulin-like receptors (KIRs) are immune receptors that are expressed by NK cells. KIRs may contain ITAMs or immunoreceptor tyrosine-based inhibitory motifs (ITIMs) to modulate the activation of immune cells. As such, KIR-based synthetic receptors may provide a promising alternative to traditional CD3z-based CARs for the treatment of disease with reduced toxicity, and a lower potential for exhaustion. Additional approaches include tuning the response by combining KIR domains or portions thereof with CD3z domains or portions thereof to generate the recombinant immune cell.

### SUMMARY OF THE INVENTION

Disclosed herein, in various embodiments, is a method of activating a recombinant immune cell expressing a synthetic receptor, comprising (a) an antigen-binding domain; a transmembrane domain; and an intracellular signaling domain comprising a sequence of 10-29 amino acids derived from killer cell immunoglobulin-like receptor 2DL4 (KIR2DL4); wherein the method comprises binding an antigen to the antigen-binding domain, thereby activating the recombinant immune cell.

In some embodiments, the intracellular signaling domain comprises or consists of an amino acid sequence of SEQ ID NO: 8 or 9. In some embodiments, the intracellular signaling domain comprises or consists of an amino acid sequence of SEQ ID NO:8. In some embodiments, the intracellular signaling domain comprises or consists of an amino acid sequence of SEQ ID NO:9. In some embodiments, the intracellular signaling domain further comprises a costimulatory domain comprising an amino acid sequence selected from any one of SEQ ID NOs: 12-16. In some embodiments, the intracellular signaling domain further comprises a costimulatory domain comprising an amino acid of SEQ ID NOs: 12. In some embodiments, the intracellular signaling domain further comprises a costimulatory domain comprising an amino acid of SEQ ID NOs: 13. In some embodiments, the intracellular signaling domain further comprises a costimulatory domain comprising an amino acid of SEQ ID NOs: 14. In some embodiments, the intracellular signaling domain further comprises a costimulatory domain comprising an amino acid of SEQ ID NOs: 15. In some embodiments, the intracellular signaling domain further comprises a costimulatory domain comprising an amino acid of SEQ ID NOs: 16.

In some embodiments, the transmembrane domain comprises or consists of an amino acid sequence of SEQ ID NO: 1, 2 or 3. In some embodiments, the transmembrane domain comprises or consists of an amino acid sequence of SEQ ID NO:1. In some embodiments, the transmembrane domain comprises or consists of an amino acid sequence of SEQ ID NO:2. In some embodiments, the transmembrane domain comprises or consists of an amino acid sequence of SEQ ID NO:3.

In some embodiments, the synthetic receptor further comprises a hinge region interposed between the antigen-binding domain and the transmembrane domain. In some embodiments, the hinge region comprises an amino acid sequence of SEQ ID NO: 4 or 5. In some embodiments, the hinge region comprises an amino acid sequence of SEQ ID NO:4. In some embodiments, the hinge region comprises an amino acid sequence of SEQ ID NO:5.

In some embodiments, the synthetic receptor comprises an amino acid sequence having at least 90% sequence identity with an amino acid sequence selected from any one of SEQ ID NOs: 17-34. In some embodiments, the synthetic receptor comprises an amino acid sequence having at least 90% sequence identity with the amino acid sequence of SEQ ID NO:17. In some embodiments, the synthetic receptor comprises an amino acid sequence having at least 90% sequence identity with the amino acid sequence of SEQ ID NO:18. In some embodiments, the synthetic receptor comprises an amino acid sequence having at least 90% sequence identity with the amino acid sequence of SEQ ID NO:19. In some embodiments, the synthetic receptor comprises an amino acid sequence having at least 90% sequence identity with the amino acid sequence of SEQ ID NO:20. In some embodiments, the synthetic receptor comprises an amino acid sequence having at least 90% sequence identity with the amino acid sequence of SEQ ID NO:21. In some embodiments, the synthetic receptor comprises an amino acid sequence having at least 90% sequence identity with the amino acid sequence of SEQ ID NO:22. In some embodiments, the synthetic receptor comprises an amino acid sequence having at least 90% sequence identity with the amino acid sequence of SEQ ID NO:23. In some embodiments, the synthetic receptor comprises an amino acid sequence having at least 90% sequence identity with the amino acid sequence of SEQ ID NO:24. In some embodiments, the synthetic receptor comprises an amino acid sequence having at least 90% sequence identity with the amino acid sequence of SEQ ID NO:25. In some embodiments, the synthetic receptor comprises an amino acid sequence having at least 90% sequence identity with the amino acid sequence of SEQ ID NO:26. In some embodiments, the synthetic receptor comprises an amino acid sequence having at least 90% sequence identity with the amino acid sequence of SEQ ID NO:27. In some embodiments, the synthetic receptor comprises an amino acid sequence having at least 90% sequence identity with the amino acid sequence of SEQ ID NO:28. In some embodiments, the synthetic receptor comprises an amino acid sequence having at least 90% sequence identity with the amino acid sequence of SEQ ID NO:29. In some embodiments, the synthetic receptor comprises an amino acid sequence having at least 90% sequence identity with the amino acid sequence of SEQ ID NO:30. In some embodiments, the synthetic receptor comprises an amino acid sequence having at least 90% sequence identity with the amino acid sequence of SEQ ID NO:31. In some embodiments, the synthetic receptor comprises an amino acid sequence having at least 90% sequence identity with the amino acid sequence of SEQ ID NO:32. In some embodiments, the synthetic receptor comprises an amino acid sequence having at least 90% sequence identity with the amino acid sequence of SEQ ID NO:33. In some embodiments, the synthetic receptor comprises an amino acid sequence having at least 90% sequence identity with the amino acid sequence of SEQ ID NO:34.

In some embodiments, the antigen-binding domain comprises an antibody or functional fragment thereof. In some embodiments, the antigen-binding domain comprises a single-chain variable fragment (scFv). In some embodiments, the antigen-binding domain comprises a minimal active antibody fragment. In some embodiments, the antigen-binding domain comprises a single domain antibody. In some embodiments, the antigen-binding domain comprises a single light chain variable domain. In some embodiments, the antigen-binding domain comprises a single heavy chain variable domain. In some embodiments, the antigen-binding domain comprises a nanobody.

In some embodiments, the recombinant immune cell is a T cell, a Natural Killer (NK) cell, or a Natural Killer-type T (NK-T) cell. In some embodiments, the recombinant immune cell is a T cell. In some embodiments, the recombinant immune cell is an NK cell. In some embodiments, the recombinant immune cell is an NK-T cell.

In some embodiments, the antigen is a tumor-associated antigen. In some embodiments, the antigen is a whole protein or a fragment thereof selected from the list consisting of CD19, mesothelin, CD123, BCMA, GD2, CD30, GPC3, CD22, HER2, CD20, EGFR, Flt3, CD33, Muc-16, CS1, and a tumor neoantigen. In some embodiments, the antigen is CD19 or a fragment thereof. In some embodiments, the antigen is mesothelin or a fragment thereof. In some embodiments, the antigen is CD123 or a fragment thereof. In some embodiments, the antigen is BCMA or a fragment thereof. In some embodiments, is GD2 or a fragment thereof. In some embodiments, the antigen is CD30 or a fragment thereof. In some embodiments, the antigen is GPC3 or a fragment thereof. In some embodiments, the antigen is CD22 or a fragment thereof. In some embodiments, the antigen is HER2 or a fragment thereof. In some embodiments, the antigen is CD20 or a fragment thereof. In some embodiments, the antigen is EGFR or a fragment thereof. In some embodiments, antigen is Flt3 or a fragment thereof. In some embodiments, the antigen is CD33 or a fragment thereof. In some embodiments, the antigen is Muc-16 or a fragment thereof. In some embodiments, the antigen is CS1 or a fragment thereof. In some embodiments, the antigen is a tumor neoantigen. In some embodiments, the antigen is derived from an infectious agent. In some embodiments, the infectious agent is a bacterium, a virus, a fungus, or a parasite.

Also disclosed herein, in various embodiments, is a recombinant immune cell, comprising: an expressed synthetic receptor, the receptor comprising: (a) an antigen-binding domain; a transmembrane domain; and an intracellular signaling domain comprising or consisting of a sequence of 10-29 amino acids derived from killer cell immunoglobulin-like receptor 2DL4 (KIR2DL4); wherein the recombinant immune cell is activated by binding a cognate antigen to the antigen-binding domain.

In some embodiments, the intracellular signaling domain comprises or consists of an amino acid sequence of SEQ ID NO: 8 or 9. In some embodiments, the intracellular signaling domain comprises or consists of an amino acid sequence of SEQ ID NO:8. In some embodiments, the intracellular signaling domain comprises or consists of an amino acid sequence of SEQ ID NO:9. In some embodiments, the intracellular signaling domain further comprises a costimulatory domain comprising an amino acid sequence selected from any one of SEQ ID NOs: 12-16. In some embodiments, the intracellular signaling domain further comprises a costimulatory domain comprising an amino acid of SEQ ID NOs: 12. In some embodiments, the intracellular signaling domain further comprises a costimulatory domain comprising an amino acid of SEQ ID NOs: 13. In some embodiments, the intracellular signaling domain further comprises a costimulatory domain comprising an amino acid of SEQ ID NOs: 14. In some embodiments, the intracellular signaling domain further comprises a costimulatory domain comprising an amino acid of SEQ ID NOs: 15. In some embodiments, the intracellular signaling domain further comprises a costimulatory domain comprising an amino acid of SEQ ID NOs: 16.

In some embodiments, the transmembrane domain comprises or consists of an amino acid sequence of SEQ ID NO: 1, 2 or 3. In some embodiments, the transmembrane domain comprises or consists of an amino acid sequence of SEQ ID NO:1. In some embodiments, the transmembrane domain comprises or consists of an amino acid sequence of SEQ ID NO:2. In some embodiments, the transmembrane domain comprises or consists of an amino acid sequence of SEQ ID NO:3.

In some embodiments, the synthetic receptor further comprises a hinge region interposed between the antigen-binding domain and the transmembrane domain. In some embodiments, the hinge region comprises an amino acid sequence of SEQ ID NO: 4 or 5. In some embodiments, the hinge region comprises an amino acid sequence of SEQ ID NO:4. In some embodiments, the hinge region comprises an amino acid sequence of SEQ ID NO:5.

In some embodiments, the synthetic receptor comprises an amino acid sequence having at least 90% sequence identity with an amino acid sequence selected from any one of SEQ ID NOs: 17-34. In some embodiments, the synthetic receptor comprises an amino acid sequence having at least 90% sequence identity with the amino acid sequence of SEQ ID NO:17. In some embodiments, the synthetic receptor comprises an amino acid sequence having at least 90% sequence identity with the amino acid sequence of SEQ ID NO:18. In some embodiments, the synthetic receptor comprises an amino acid sequence having at least 90% sequence identity with the amino acid sequence of SEQ ID NO:19. In some embodiments, the synthetic receptor comprises an amino acid sequence having at least 90% sequence identity with the amino acid sequence of SEQ ID NO:20. In some embodiments, the synthetic receptor comprises an amino acid sequence having at least 90% sequence identity with the amino acid sequence of SEQ ID NO:21. In some embodiments, the synthetic receptor comprises an amino acid sequence having at least 90% sequence identity with the amino acid sequence of SEQ ID NO:22. In some embodiments, the synthetic receptor comprises an amino acid sequence having at least 90% sequence identity with the amino acid sequence of SEQ ID NO:23. In some embodiments, the synthetic receptor comprises an amino acid sequence having at least 90% sequence identity with the amino acid sequence of SEQ ID NO:24. In some embodiments, the synthetic receptor comprises an amino acid sequence having at least 90% sequence identity with the amino acid sequence of SEQ ID NO:25. In some embodiments, the synthetic receptor comprises an amino acid sequence having at least 90% sequence identity with the amino acid sequence of SEQ ID NO:26. In some embodiments, the synthetic receptor comprises an amino acid sequence having at least 90% sequence identity with the amino acid sequence of SEQ ID NO:27. In some embodiments, the synthetic receptor comprises an amino acid sequence having at least 90% sequence identity with the amino acid sequence of SEQ ID NO:28. In some embodiments, the synthetic receptor comprises an amino acid sequence having at least 90% sequence identity with the amino acid sequence of SEQ ID NO:29. In some embodiments, the synthetic receptor comprises an amino acid sequence having at least 90% sequence identity with the amino acid sequence of SEQ ID NO:30. In some embodiments, the synthetic receptor comprises an amino acid sequence having at least 90% sequence identity with the amino acid sequence of SEQ ID NO:31. In some embodiments, the synthetic receptor comprises an amino acid sequence having at least 90% sequence identity with the amino acid sequence of SEQ ID NO:32. In some embodiments, the synthetic receptor comprises an amino acid sequence having at least 90% sequence identity with the amino acid sequence of SEQ ID NO:33. In some embodiments, the synthetic receptor comprises an amino acid sequence having at least 90% sequence identity with the amino acid sequence of SEQ ID NO:34.In some embodiments, the antigen-binding domain comprises an antibody or functional fragment thereof. In some embodiments, the antigen-binding domain comprises a single-chain variable fragment (scFv).

In some embodiments, the antigen-binding domain comprises a minimal active antibody fragment. In some embodiments, the antigen-binding domain comprises a single domain antibody. In some embodiments, the antigen-binding domain comprises a single light chain variable domain. In some embodiments, the antigen-binding domain comprises a single heavy chain variable domain. In some embodiments, the antigen-binding domain comprises a nanobody.

In some embodiments, the recombinant immune cell is a T cell, an NK cell, or an NK-T cell. In some embodiments, the recombinant immune cell is a T cell. In some embodiments, the recombinant immune cell is an NK cell. In some embodiments, the recombinant immune cell is an NK-T cell.

Also disclosed herein, in various embodiments, is a method of treating a disease in a subject in need thereof, the method comprising administering to the subject the recombinant immune cell disclosed herein. In some embodiments, the subject is a mammal. In some embodiments, the subject is a human. In some embodiments, the recombinant cell is autologous to the subject. In some embodiments, the recombinant cell is allogenic to the subject.

In some embodiments, the disease is a cancer. In some embodiments, the cancer is a hematopoietic malignancy. In some embodiments, the cancer is a solid tumor. In some embodiments, the antigen is a whole protein or a fragment thereof selected from the list consisting of CD19, mesothelin, CD123, BCMA, GD2, CD30, GPC3, CD22, HER2, CD20, EGFR, Flt3, CD33, Muc-16, CS1, and a tumor neoantigen. In some embodiments, the antigen is CD19 or a fragment thereof. In some embodiments, the antigen is mesothelin or a fragment thereof. In some embodiments, the antigen is CD123 or a fragment thereof. In some embodiments, the antigen is BCMA or a fragment thereof. In some embodiments, the antigen is GD2 or a fragment thereof. In some embodiments, the antigen is CD30 or a fragment thereof. In some embodiments, the antigen is GPC3 or a fragment thereof. In some embodiments, the antigen is CD22 or a fragment thereof. In some embodiments, the antigen is HER2 or a fragment thereof. In some embodiments, the antigen is CD20 or a fragment thereof. In some embodiments, the antigen is EGFR or a fragment thereof. In some embodiments, the antigen is Flt3 or a fragment thereof. In some embodiments, the antigen is CD33 or a fragment thereof. In some embodiments, the antigen is Muc-16 or a fragment thereof. In some embodiments, the antigen is CS1 or a fragment thereof. In some embodiments, the antigen is a tumor neoantigen.

In some embodiments, the disease is an infectious disease caused by an infectious agent. In some embodiments, the infectious agent is a bacterium, a virus, a fungus, or a parasite.

Also provided herein, in various embodiments, is a method of activating a recombinant immune cell expressing a synthetic receptor, comprising: (a) means for binding an antigen of interest; (b) a transmembrane domain; and (c) an intracellular signaling domain comprising a sequence of 10-29 amino acids derived from killer cell immunoglobulin-like receptor 2DL4 (KIR2DL4); wherein the method comprises binding the antigen of interest, thereby activating the recombinant immune cell.

Also provided herein, in various embodiments, is a recombinant immune cell, comprising: an expressed synthetic receptor, the receptor comprising: (a) means for binding an antigen of interest; (b) a transmembrane domain; and (c) an intracellular signaling domain comprising or consisting of a sequence of 10-29 amino acids derived from killer cell immunoglobulin-like receptor 2DL4 (KIR2DL4); wherein the recombinant immune cell is activated by binding a cognate antigen to the antigen-binding domain.

### BRIEF DESCRIPTION OF THE DRAWINGS

FIGS. 1A-1C show activation of Jurkat T reporter cells expressing the indicated recombinant receptors without (−Raji) or with (+Raji) exposure to cancer cells. Data are presented as scatter plots of flow cytometry analysis with mCherry, a surrogate measurement of receptor transgene expression on the X axis and mCitrine, a gene expression reporter of T cell activation on the Y axis. FIG. 1A shows cells expressing a synthetic receptor with no antigen-binding domain. FIG. 1B shows cells expressing a control anti-CD19 CAR containing a CD3z intracellular signaling domain (ICD). FIG. 1C shows cells expressing a synthetic receptor comprising a full-length KIR2DL4 ICD. The quadrants indicate gates denoting cells expressing the recombinant receptors (2 quandrants on the right) and the activated cells (2 quadrants on the top).

FIG. 2 shows a bar graph (left) showing log-2 fold changes of mCitrine reporter expression as a measure of T cell activation and diagrams of synthetic receptor construction (right) indicating N-terminal and/or C-terminal truncations of the KIR2DL4 ICD.

FIG. 3 shows a bar graph of log-2 fold-change (log2FC) of activation performance on the Y axis. The X axis shows synthetic receptors including negative control CAR with no ICD (no ICD CAR), an anti-CD19 positive control CAR with a CD3z ICD. The additional constructs expressed (1-5) comprise a full-length KIR2DL4 transmembrane domain (TM) and ICD (#1), KIR2DL4 TM with KIR2DL4 ICD truncation A (#2), KIR2DL4 TM with KIR2DL4 ICD ITIM T37A Y38F substitutions (#3), KIR2DL4 TM with no ICD (#4), and CD8a TM with KIR2DL4 ICD (#5). The error bars represent standard errors.

FIGS. 4A-4C depict transduction and functional assessment of primary T cells expressing the indicated recombinant receptors with a CD19 scFv. FIG. 4A shows transduction efficiency as measured with a FITC-conjugated CD19 protein and reported as MFI. FIG. 4B shows readouts of a short-term luciferase-based cytotoxicity assay for T Cells transduced in FIG. 4A exposed to Raji target cells. FIG. 4C shows readouts of a repetitive luciferase-based cytotoxicity assay for T Cells transduced in FIG. 4A exposed to Raji target cells. Cytotoxicity was calculated by normalizing the killing in each condition to wells containing target cells alone.

FIGS. 5A and 5B depict transduction and functional assessment of primary NK cells expressing the indicated recombinant receptors with a CD19 scFv. FIG. 5A shows transduction efficiency as measured with a FITC-conjugated CD19 protein and reported as MFI. FIG. 5B shows measurement of CD107a in NK Cells transduced in FIG. 5A activated with Raji target cells.

FIGS. 6A and 6B depict activation of Jurkat T reporter cells modified to express mCitrine as a marker of NFAT-NFkB gene activation and expressing the indicated recombinant receptors with a CD19 or MSLN scFv. FIG. 6A depicts Jurkat T cell activation after exposure to CD19-expressing Raji target cells. FIG. 6B depicts Jurkat T cell activation after exposure to MSLN-expressing K562 target cells. Percentage of cells expressing mCitrine is measured by flow cytometry.

FIGS. 7A and 7B depict transduction and functional assessment of primary T cells expressing the indicated recombinant receptors with a BCMA scFv. FIG. 7A shows transduction efficiency as measured by the expression of the mScarlet fluorescent reporter protein. FIG. 7B depicts cytotoxicity of K562-BCMA⁺ target cells induced by transduced T cells from FIG. 7A after a 24-hour incubation. The cytotoxic capacity of these CAR T cells was assessed by flow cytometry.

FIGS. 8A and 8B depict transduction and functional assessment of primary T cells expressing the indicated recombinant receptors with a MSLN scFv. FIG. 8A depicts transduction efficiency as measured by the expression of the mScarlet fluorescent reporter protein. FIG. 8B depicts cytotoxicity of K562-MSLN⁺ target cells induced by transduced T cells from FIG. 8A after a 24-hour incubation. The cytotoxic capacity of these CAR T cells was assessed by flow cytometry.

### DETAILED DESCRIPTION

### I. Definitions

Unless defined otherwise, all technical and scientific terms used herein have the meaning commonly understood by one of ordinary skill in the art to which this disclosure belongs.

The terms “a,” and “an” as used herein mean “one or more” and include the plural unless the context is appropriate.

The term “antigen” refers to a molecule (e.g., peptide or protein) or immunologically active fragment thereof that is capable of eliciting an immune response. Peptide antigens are typically presented by an antigen presenting cell (APC) to an immune cell, such as a T lymphocyte (also called a T cell).

As used herein, “antigen-specific” refers to an immune response generated in a host that is specific to a given antigen. The term includes responses to antigens that are recognized by antibodies capable of binding to the antigen of interest with high affinity, and responses to antigens by T cell receptors (TCRs) that recognize and bind to a complex comprising an WIC molecule and a short peptide that is a degradation product of the antigen of interest.

The term “fusion peptide or protein” or “fused peptide or protein” refers to a recombinant protein comprising two or more proteins or peptides expressed in the same amino acid chain in sequence. The two or more protein or peptide nucleic acid coding sequences can be expressed sequentially in a single open reading frame of a vector or expression plasmid. The resulting peptide or protein thus comprises a single amino acid chain with two or more proteins of interest connected via end-to-end fusion at the N- or C-termini. As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

An “effective amount” of an agent, e.g., cells or a pharmaceutical composition, in the context of administration, refers to an amount effective, at dosages/amounts and for periods of time necessary, to achieve a desired result, such as a therapeutic or prophylactic result.

The term percent “identity” or “sequence identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As used herein, the term “recombinant” refers to a protein, cell, or organism that does not exist in nature. The term is used interchangeably with “engineered” or “modified.”

As used herein, the terms “subject” or “patient” are used interchangeably, and refer to an organism to which a recombinant immune cell expressing a synthetic receptor is administered. A subject can be a mammalian subject, e.g., a human subject. In some embodiments, the subject has a disease, condition, or disorder. In some embodiments, the disease, condition, or disorder is characterized by the presence of a specific antigen that can be targeted by a synthetic receptor.

As used herein, to “suppress” a function or activity is to reduce the function or activity when compared to otherwise same conditions except for a condition or parameter of interest, or alternatively, as compared to another condition. For example, a recombinant immune cell which suppresses tumor growth reduces the rate of growth of the tumor compared to the rate of growth of the tumor in the absence of the recombinant immune cell.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

A “therapeutically effective amount” of an agent, e.g., a cells or a pharmaceutical composition, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

### II. Synthetic Receptors

Disclosed herein is a recombinant immune cell and method of activating the same through the activation of a synthetic receptor. A “synthetic receptor” is a fusion protein comprising portions derived from multiple, distinct proteins, that elicits a response when bound by a ligand. In some embodiments, the synthetic receptor is a chimeric antigen receptor (CAR). A “chimeric antigen receptor” is a synthetic receptor that mimics the activity of a TCR and can induce antigen-specific immune cell activation upon binding to an antigen. A CAR recognizes a cell-surface antigen independent of human leukocyte antigen (HLA) and employs one or more signaling molecules to activate genetically-modified immune cells for killing, proliferation, and cytokine production (Jena et al., 2010). CARs may be employed to impart the specificity of a monoclonal antibody onto an immune cell, thereby allowing a large number of specific immune cells to be generated, for example, for use in adoptive cell therapy. In some embodiments, the CAR comprises an intracellular signaling domain, a hinge domain, a transmembrane domain, and a targeting domain comprising a ligand-binding region. The specificity of other CAR designs may be derived from ligands of receptors (e.g., peptides) or from pattern-recognition receptors, such as Dectins. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

Exemplary antigen receptors, including CARs, and methods for engineering and introducing such receptors into cells, include those described, for example, in international patent application publication numbers WO2000/14257, WO2013/126726, WO2012/129514, WO2014/031687, WO2013/166321, WO2013/071154, WO2013/123061, U.S. patent application publication numbers US2002/131960, US2013/287748, US2013/0149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4):388-398; Davila et al. (2013) PLoS ONE 8(4):e61338; Turtle et al., Curr. Opin. Immunol., 2012 October; 24(5): 633-39; Wu et al., Cancer, 2012 March 18(2):160-75.

IIa. Intracellular Signaling Domain

The targeting domain generally is linked to one or more intracellular signaling components, such as signaling components that mimic activation through an antigen receptor complex, such as a TCR complex, in the case of a CAR, and/or signal via another cell surface receptor. Thus, in some embodiments, the targeting (e.g., antigen-binding) and transmembrane domains are linked to one or more intracellular signaling domains. In some embodiments, the transmembrane domain is fused to the targeting domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

In some embodiments, the intracellular signaling domain comprises one or more immunoreceptor tyrosine-based inhibitory motifs (ITIMs). An ITIM is a protein domain that is found on the intracellular cytoplasmic domains of many immune receptors. ITIMs can inhibit cellular activation by immunoreceptor tyrosine-based activating motifs (ITAMs). In some embodiments, the intracellular signaling domain comprising one or more ITIMs does not comprise an ITAM.

In various embodiments, the intracellular signaling domain of the synthetic receptor disclosed herein comprises an amino acid sequence of the intracellular signaling domain of KIR2DL4. Exemplary intracellular domains of Killer cell immunoglobulin-like receptor 2DL4 (KIR2DL4) or derived from KIR2DL4 are provided in Table 1. In various embodiments, the synthetic receptor comprises an intracellular signaling domain comprising an amino acid sequence provided in Table 1.

TABLE 1
KIR2DL4-based Intracellular Signaling Domains

| Fragment | Sequence |
|---|---|
| KIR2DL intracellular | HRWCSKKKDAAVMNQEPAGHRTVNR |
| signaling domain | EDSDEQDPQEVTYAQLDHCIFTQRK |
| | ITGPSQRSKRPSTDTSVCIELPNAE |
| | PRALSPAHEHHSQALMGSSRETTAL |
| | SQTQLASSNVPAAGI |
| | (SEQ ID NO: 6) |
| KIR2DL intracellular | HRWCSKKKDAAVMNQEPAGHRTVNR |
| signaling domain | EDSDEQDPQEVAFAQLDHCIFTQRK |
| T37A Y38F | ITGPSQRSKRPSTDTSVCIELPNAE |
| | PRALSPAHEHHSQALMGSSRETTAL |
| | SQTQLASSNVPAAGI |
| | (SEQ ID NO: 7) |
| KIR2DL intracellular | HRWCSKKKDAAVMNQEPAGHRTVNR |
| signaling domain | EDSD |
| Truncation A | (SEQ ID NO: 8) |
| KIR2DL intracellular | HRWCSKKKDA |
| signaling domain N- | (SEQ ID NO: 9) |
| terminus | |
| KIR2DL intracellular | HRWCSKKKDAAVMNQEPAGHRTVNR |
| signaling domain | EDSDEQDPQEVAFAQLDHCIFTQR |
| Truncation B ITIM | (SEQ ID NO: 10) |
| mut | |
| KIR2DL intracellular | HRWCSKKKDAAVMNQEPAGHRTVNR |
| signaling domain | EDSDEQDPQEVAFAQLDHCIFTQRK |
| Truncation C ITIM | ITGPSQRS (SEQ ID NO: 11) |
| mut | |

In some embodiments, the intracellular signaling domain comprises or consists of an amino acid sequence corresponding to the sequence of amino acids 263-377 of full-length KIR2DL4 (SEQ ID NO:6). In some embodiments, the intracellular signaling domain comprises amino acid substitutions T37A and/or Y38F within the amino acid sequence of SED ID NO:6. In embodiments in which the length of the intracellular signaling domain is less than the length of SEQ ID NO:6, percent identity is defined with respect to the identity of the signaling domain to SEQ ID NO:6 over the length of the intracellular signaling domain.

In some embodiments, the intracellular signaling domain comprises or consists of a segment having an amino acid sequence at least 70%, 75%, 80%, 85%, 90%, 95%, 96%, 97%, 98%, 99%, 99.5%, 99.9%, or 100% identical to SEQ ID NO:6. In some embodiments, the intracellular signaling domain comprises or consists of a segment having an amino acid sequence at least 70% identical to SEQ ID NO:6. In some embodiments, the intracellular signaling domain comprises or consists of a segment having an amino acid sequence at least 75% identical to SEQ ID NO:6. In some embodiments, the intracellular signaling domain comprises or consists of a segment having an amino acid sequence at least 80% identical to SEQ ID NO:6. In some embodiments, the intracellular signaling domain comprises or consists of a segment having an amino acid sequence at least 85% identical to SEQ ID NO:6. In some embodiments, the intracellular signaling domain comprises or consists of a segment having an amino acid sequence at least 90% identical to SEQ ID NO:6. In some embodiments, the intracellular signaling domain comprises or consists of a segment having an amino acid sequence at least 95% identical to SEQ ID NO:6. In some embodiments, the intracellular signaling domain comprises or consists of a segment having an amino acid sequence at least 96% identical to SEQ ID NO:6. In some embodiments, the intracellular signaling domain comprises or consists of a segment having an amino acid sequence at least 97% identical to SEQ ID NO:6. In some embodiments, the intracellular signaling domain comprises or consists of a segment having an amino acid sequence at least 98% identical to SEQ ID NO:6. In some embodiments, the intracellular signaling domain comprises or consists of a segment having an amino acid sequence at least 99% identical to SEQ ID NO:6. In some embodiments, the intracellular signaling domain comprises or consists of a segment having an amino acid sequence at least 99.5% identical to SEQ ID NO:6. In some embodiments, the intracellular signaling domain comprises or consists of a segment having an amino acid sequence at least 99.9% identical to SEQ ID NO:6. In some embodiments, the intracellular signaling domain comprises or consists of a segment having an amino acid sequence of SEQ ID NO:6.

In some embodiments, the intracellular signaling domain comprises or consists of a segment having an amino acid sequence at least 70%, 75%, 80%, 85%, 90%, 95%, 96%, 97%, 98%, 99%, 99.5%, 99.9%, or 100% identical to SEQ ID NO:7. In some embodiments, the intracellular signaling domain comprises or consists of a segment having an amino acid sequence at least 70% identical to SEQ ID NO:7. In some embodiments, the intracellular signaling domain comprises or consists of a segment having an amino acid sequence at least 75% identical to SEQ ID NO:7. In some embodiments, the intracellular signaling domain comprises or consists of a segment having an amino acid sequence at least 80% identical to SEQ ID NO:7. In some embodiments, the intracellular signaling domain comprises or consists of a segment having an amino acid sequence at least 85% identical to SEQ ID NO:7. In some embodiments, the intracellular signaling domain comprises or consists of a segment having an amino acid sequence at least 90% identical to SEQ ID NO:7. In some embodiments, the intracellular signaling domain comprises or consists of a segment having an amino acid sequence at least 95% identical to SEQ ID NO:7. In some embodiments, the intracellular signaling domain comprises or consists of a segment having an amino acid sequence at least 96% identical to SEQ ID NO:7. In some embodiments, the intracellular signaling domain comprises or consists of a segment having an amino acid sequence at least 97% identical to SEQ ID NO:7. In some embodiments, the intracellular signaling domain comprises or consists of a segment having an amino acid sequence at least 98% identical to SEQ ID NO:7. In some embodiments, the intracellular signaling domain comprises or consists of a segment having an amino acid sequence at least 99% identical to SEQ ID NO:7. In some embodiments, the intracellular signaling domain comprises or consists of a segment having an amino acid sequence at least 99.5% identical to SEQ ID NO:7. In some embodiments, the intracellular signaling domain comprises or consists of a segment having an amino acid sequence at least 99.9% identical to SEQ ID NO:7. In some embodiments, the intracellular signaling domain comprises or consists of a segment having an amino acid sequence of SEQ ID NO:7.

In some embodiments, the intracellular signaling domain comprises or consists of a segment having an amino acid sequence at least 70%, 75%, 80%, 85%, 90%, 95%, 96%, 97%, 98%, 99%, 99.5%, 99.9%, or 100% identical to SEQ ID NO:8. In some embodiments, the intracellular signaling domain comprises or consists of a segment having an amino acid sequence at least 70% identical to SEQ ID NO:8. In some embodiments, the intracellular signaling domain comprises or consists of a segment having an amino acid sequence at least 75% identical to SEQ ID NO:8. In some embodiments, the intracellular signaling domain comprises or consists of a segment having an amino acid sequence at least 80% identical to SEQ ID NO:8. In some embodiments, the intracellular signaling domain comprises or consists of a segment having an amino acid sequence at least 85% identical to SEQ ID NO:8. In some embodiments, the intracellular signaling domain comprises or consists of a segment having an amino acid sequence at least 90% identical to SEQ ID NO:8. In some embodiments, the intracellular signaling domain comprises or consists of a segment having an amino acid sequence at least 95% identical to SEQ ID NO:8. In some embodiments, the intracellular signaling domain comprises or consists of a segment having an amino acid sequence at least 96% identical to SEQ ID NO:8. In some embodiments, the intracellular signaling domain comprises or consists of a segment having an amino acid sequence at least 97% identical to SEQ ID NO:8. In some embodiments, the intracellular signaling domain comprises or consists of a segment having an amino acid sequence at least 98% identical to SEQ ID NO:8. In some embodiments, the intracellular signaling domain comprises or consists of a segment having an amino acid sequence at least 99% identical to SEQ ID NO:8. In some embodiments, the intracellular signaling domain comprises or consists of a segment having an amino acid sequence at least 99.5% identical to SEQ ID NO:8. In some embodiments, the intracellular signaling domain comprises or consists of a segment having an amino acid sequence at least 99.9% identical to SEQ ID NO:8. In some embodiments, the intracellular signaling domain comprises or consists of a segment having an amino acid sequence of SEQ ID NO:8.

In some embodiments, the intracellular signaling domain comprises or consists of a segment having an amino acid sequence at least 70%, 75%, 80%, 85%, 90%, 95%, 96%, 97%, 98%, 99%, 99.5%, 99.9%, or 100% identical to SEQ ID NO:9. In some embodiments, the intracellular signaling domain comprises or consists of a segment having an amino acid sequence at least 70% identical to SEQ ID NO:9. In some embodiments, the intracellular signaling domain comprises or consists of a segment having an amino acid sequence at least 75% identical to SEQ ID NO:9. In some embodiments, the intracellular signaling domain comprises or consists of a segment having an amino acid sequence at least 80% identical to SEQ ID NO:9. In some embodiments, the intracellular signaling domain comprises or consists of a segment having an amino acid sequence at least 85% identical to SEQ ID NO:9. In some embodiments, the intracellular signaling domain comprises or consists of a segment having an amino acid sequence at least 90% identical to SEQ ID NO:9. In some embodiments, the intracellular signaling domain comprises or consists of a segment having an amino acid sequence at least 95% identical to SEQ ID NO:9. In some embodiments, the intracellular signaling domain comprises or consists of a segment having an amino acid sequence at least 96% identical to SEQ ID NO:9. In some embodiments, the intracellular signaling domain comprises or consists of a segment having an amino acid sequence at least 97% identical to SEQ ID NO:9. In some embodiments, the intracellular signaling domain comprises or consists of a segment having an amino acid sequence at least 98% identical to SEQ ID NO:9. In some embodiments, the intracellular signaling domain comprises or consists of a segment having an amino acid sequence at least 99% identical to SEQ ID NO:9. In some embodiments, the intracellular signaling domain comprises or consists of a segment having an amino acid sequence at least 99.5% identical to SEQ ID NO:9. In some embodiments, the intracellular signaling domain comprises or consists of a segment having an amino acid sequence at least 99.9% identical to SEQ ID NO:9. In some embodiments, the intracellular signaling domain comprises or consists of a segment having an amino acid sequence of SEQ ID NO:9.

In some embodiments, the intracellular signaling domain comprises or consists of a segment having an amino acid sequence at least 70%, 75%, 80%, 85%, 90%, 95%, 96%, 97%, 98%, 99%, 99.5%, 99.9%, or 100% identical to SEQ ID NO:10. In some embodiments, the intracellular signaling domain comprises or consists of a segment having an amino acid sequence at least 70% identical to SEQ ID NO:10. In some embodiments, the intracellular signaling domain comprises or consists of a segment having an amino acid sequence at least 75% identical to SEQ ID NO:10. In some embodiments, the intracellular signaling domain comprises or consists of a segment having an amino acid sequence at least 80% identical to SEQ ID NO:10. In some embodiments, the intracellular signaling domain comprises or consists of a segment having an amino acid sequence at least 85% identical to SEQ ID NO:10. In some embodiments, the intracellular signaling domain comprises or consists of a segment having an amino acid sequence at least 90% identical to SEQ ID NO:10. In some embodiments, the intracellular signaling domain comprises or consists of a segment having an amino acid sequence at least 95% identical to SEQ ID NO:10. In some embodiments, the intracellular signaling domain comprises or consists of a segment having an amino acid sequence at least 96% identical to SEQ ID NO:10. In some embodiments, the intracellular signaling domain comprises or consists of a segment having an amino acid sequence at least 97% identical to SEQ ID NO:10. In some embodiments, the intracellular signaling domain comprises or consists of a segment having an amino acid sequence at least 98% identical to SEQ ID NO:10. In some embodiments, the intracellular signaling domain comprises or consists of a segment having an amino acid sequence at least 99% identical to SEQ ID NO:10. In some embodiments, the intracellular signaling domain comprises or consists of a segment having an amino acid sequence at least 99.5% identical to SEQ ID NO:10. In some embodiments, the intracellular signaling domain comprises or consists of a segment having an amino acid sequence at least 99.9% identical to SEQ ID NO:10. In some embodiments, the intracellular signaling domain comprises or consists of a segment having an amino acid sequence of SEQ ID NO:10.

In some embodiments, the intracellular signaling domain comprises or consists of a segment having an amino acid sequence at least 70%, 75%, 80%, 85%, 90%, 95%, 96%, 97%, 98%, 99%, 99.5%, 99.9%, or 100% identical to SEQ ID NO:11. In some embodiments, the intracellular signaling domain comprises or consists of a segment having an amino acid sequence at least 70% identical to SEQ ID NO:11. In some embodiments, the intracellular signaling domain comprises or consists of a segment having an amino acid sequence at least 75% identical to SEQ ID NO:11. In some embodiments, the intracellular signaling domain comprises or consists of a segment having an amino acid sequence at least 80% identical to SEQ ID NO:11. In some embodiments, the intracellular signaling domain comprises or consists of a segment having an amino acid sequence at least 85% identical to SEQ ID NO:11. In some embodiments, the intracellular signaling domain comprises or consists of a segment having an amino acid sequence at least 90% identical to SEQ ID NO:11. In some embodiments, the intracellular signaling domain comprises or consists of a segment having an amino acid sequence at least 95% identical to SEQ ID NO:11. In some embodiments, the intracellular signaling domain comprises or consists of a segment having an amino acid sequence at least 96% identical to SEQ ID NO:11. In some embodiments, the intracellular signaling domain comprises or consists of a segment having an amino acid sequence at least 97% identical to SEQ ID NO:11. In some embodiments, the intracellular signaling domain comprises or consists of a segment having an amino acid sequence at least 98% identical to SEQ ID NO:11. In some embodiments, the intracellular signaling domain comprises or consists of a segment having an amino acid sequence at least 99% identical to SEQ ID NO:11. In some embodiments, the intracellular signaling domain comprises or consists of a segment having an amino acid sequence at least 99.5% identical to SEQ ID NO:11. In some embodiments, the intracellular signaling domain comprises or consists of a segment having an amino acid sequence at least 99.9% identical to SEQ ID NO:11. In some embodiments, the intracellular signaling domain comprises or consists of a segment having an amino acid sequence of SEQ ID NO:11.

In some embodiments, the intracellular signaling domain comprises or consists of a sequence of between 10 and 29 amino acids. In some embodiments, the intracellular signaling domain comprises or consists of a sequence corresponding to the amino acid sequence of amino acids 263-272 of full-length KIR2DL4, or a sequence having a requisite percent identity to that sequence. In some embodiments, the percent identity is 90%, 95%, 96%, 97%, 97.5%, 98%, 98.5%, 99%, 99.5%. In some embodiments, the intracellular signaling domain comprises or consists of a sequence corresponding to the amino acid sequence of amino acids 263-291 of full-length KIR2DL4, or a sequence having a requisite percent identity to that sequence. In some embodiments, the percent identity is 90%, 95%, 96%, 97%, 97.5%, 98%, 98.5%, 99%, 99.5%.

### KIRs

KIRs are a large family of receptors present on certain subsets of lymphocytes, including NK cells. The nomenclature for KIRs is based upon the number of extracellular domains (KIR2D or KIR3D) and whether the cytoplasmic tail is either long (KIR2DL or KIR3DL) or short (KIR2DS or KIR3DS). Within humans, the presence or absence of a given KIR is variable from one NK cell to another within the NK population present in a single individual. Within the human population there is also a relatively high level of polymorphism of the KIR molecules, with certain KIR molecules being present in some, but not all individuals. Certain KIR gene products cause stimulation of lymphocyte activity when bound to an appropriate ligand. The confirmed stimulatory KIRs all have a short cytoplasmic tail with a charged transmembrane residue that associates with an adapter molecule having an ITAM Other KIR gene products are inhibitory in nature. All confirmed inhibitory KIRs have a long cytoplasmic tail and appear to interact with different subsets of HLA antigens depending upon the KIR subtype. Inhibitory KIRs display in their intracytoplasmic portion one or several ITIMs that recruit phosphatases. The known inhibitory KIR receptors include members of the KIR2DL and KIR3DL subfamilies. KIR receptors having two Ig domains (KIR2D) identify HLA-C allotypes: KIR2DL2 (formerly designated p58.2) or the closely related gene product KIR2DL3 recognizes an epitope shared by group 2 HLA-C allotypes (Cw1, 3, 7, and 8), whereas KIR2DL1 (p58.1) recognizes an epitope shared by the reciprocal group 1 HLA-C allotypes (Cw2, 4, 5, and 6). The recognition by KIR2DL1 is dictated by the presence of a Lys residue at position 80 of HLA-C alleles. KIR2DL2 and KIR2DL3 recognition is dictated by the presence of an Asn residue at position 80. Importantly the great majority of HLA-C alleles have either an Asn or a Lys residue at position 80. One KIR with three Ig domains, KIR3DL1 (p70), recognizes an epitope shared by HLA-Bw4 alleles. Finally, a homodimer of molecules with three Ig domains KIR3DL2 (p140) recognizes HLA-A3 and -A11.

Although inhibitory KIRs and other class-I inhibitory receptors (Moretta et al, 1997; Valiante et al, 1997a; Lanier, 1998) may be co-expressed by NK cells, in any given individual's NK repertoire there are cells that express a single KIR and thus, the corresponding NK cells are blocked only by cells expressing a specific class I allele group.

### Co-Stimulatory Domains

In various embodiments, the intracellular signaling domain further comprises a costimulatory domain. Costimulatory domains can provide additional signaling inputs that support activation of immune cells in response to activation of the synthetic receptor. Additional details on functions of costimulatory domains is provided in Weinkove et al., Clin Transl Immunology. 2019; 8(5):e1049.

In some embodiments, the costimulatory domain comprises an intracellular component of a TCR complex, such as a TCR CD3 chain that mediates T-cell activation and cytotoxicity, e.g., CD3 zeta (CD3z) chain. In some embodiments, the costimulatory domain comprises an intracellular component of an immune costimulatory molecule, such as CD28, 4-1BB, DAP10, or DAP12. Exemplary costimulatory domains are provided in Table 2. In various embodiments, the synthetic receptor comprises a costimulatory domain comprising an amino acid sequence provided in Table 2.

TABLE 2
Exemplary Costimulatory Domains

| Fragment | Sequence |
|---|---|
| CD3z costimulatory | RVKFSRSADAPAYKQGQNQL |
| domain | YNELNLGRREEYDVLDKRRG |
| | RDPEMGGKPRRKNPQEGLYN |
| | ELQKDKMAEAYSEIGMKGER |
| | RRGKGHDGLYQGLSTATKDT |
| | YDALHMQALPPR |
| | (SEQ ID NO: 12) |
| CD28 costimulatory | RSKRSRLLHSDYMNMTPRRP |
| domain | GPTRKHYQPYAPPRDFAAYR |
| | S (SEQ ID NO: 13) |
| 4-1BB costimulatory | KRGRKKLLYIFKQPFMRPVQ |
| domain | TTQEEDGCSCRFPEEEEGGC |
| | EL (SEQ ID NO: 14) |
| DAP10 costimulatory | LCARPRRSPAQEDGKVYINM |
| domain | PGRG |
| | (SEQ ID NO: 15) |
| DAP12 costimulatory | YFLGRLVPRGRGAAEAATRK |
| domain | QRITETESPYQELQGQRSDV |
| | YSDLNTQRPYYK |
| | (SEQ ID NO: 16) |

In some embodiments, the costimulatory domain comprises an amino acid sequence at least 70%, 75%, 80%, 85%, 90%, 95%, 96%, 97%, 98%, 99%, 99.5%, 99.9%, or 100% identical to SEQ ID NO:12. In some embodiments, the costimulatory domain comprises an amino acid sequence at least 70% identical to SEQ ID NO:12. In some embodiments, the costimulatory domain comprises an amino acid sequence at least 75% identical to SEQ ID NO:12. In some embodiments, the costimulatory domain comprises an amino acid sequence at least 80% identical to SEQ ID NO:12. In some embodiments, the costimulatory domain comprises an amino acid sequence at least 85% identical to SEQ ID NO:12. In some embodiments, the costimulatory domain comprises an amino acid sequence at least 90% identical to SEQ ID NO:12. In some embodiments, the costimulatory domain comprises an amino acid sequence at least 95% identical to SEQ ID NO:12. In some embodiments, the costimulatory domain comprises an amino acid sequence at least 96% identical to SEQ ID NO:12. In some embodiments, the costimulatory domain comprises an amino acid sequence at least 97% identical to SEQ ID NO:12. In some embodiments, the costimulatory domain comprises an amino acid sequence at least 98% identical to SEQ ID NO:12. In some embodiments, the costimulatory domain comprises an amino acid sequence at least 99% identical to SEQ ID NO:12. In some embodiments, the costimulatory domain comprises an amino acid sequence at least 99.5% identical to SEQ ID NO:12. In some embodiments, the costimulatory domain comprises an amino acid sequence at least 99.9% identical to SEQ ID NO:12. In some embodiments, the costimulatory domain comprises an amino acid sequence of SEQ ID NO:12.

In some embodiments, the costimulatory domain comprises an amino acid sequence at least 70%, 75%, 80%, 85%, 90%, 95%, 96%, 97%, 98%, 99%, 99.5%, 99.9%, or 100% identical to SEQ ID NO:13. In some embodiments, the costimulatory domain comprises an amino acid sequence at least 70% identical to SEQ ID NO:13. In some embodiments, the costimulatory domain comprises an amino acid sequence at least 75% identical to SEQ ID NO:13. In some embodiments, the costimulatory domain comprises an amino acid sequence at least 80% identical to SEQ ID NO:13. In some embodiments, the costimulatory domain comprises an amino acid sequence at least 85% identical to SEQ ID NO:13. In some embodiments, the costimulatory domain comprises an amino acid sequence at least 90% identical to SEQ ID NO:13. In some embodiments, the costimulatory domain comprises an amino acid sequence at least 95% identical to SEQ ID NO:13. In some embodiments, the costimulatory domain comprises an amino acid sequence at least 96% identical to SEQ ID NO:13. In some embodiments, the costimulatory domain comprises an amino acid sequence at least 97% identical to SEQ ID NO:13. In some embodiments, the costimulatory domain comprises an amino acid sequence at least 98% identical to SEQ ID NO:13. In some embodiments, the costimulatory domain comprises an amino acid sequence at least 99% identical to SEQ ID NO:13. In some embodiments, the costimulatory domain comprises an amino acid sequence at least 99.5% identical to SEQ ID NO:13. In some embodiments, the costimulatory domain comprises an amino acid sequence at least 99.9% identical to SEQ ID NO:13. In some embodiments, the costimulatory domain comprises an amino acid sequence of SEQ ID NO:13.

In some embodiments, the costimulatory domain comprises an amino acid sequence at least 70%, 75%, 80%, 85%, 90%, 95%, 96%, 97%, 98%, 99%, 99.5%, 99.9%, or 100% identical to SEQ ID NO:14. In some embodiments, the costimulatory domain comprises an amino acid sequence at least 70% identical to SEQ ID NO:14. In some embodiments, the costimulatory domain comprises an amino acid sequence at least 75% identical to SEQ ID NO:14. In some embodiments, the costimulatory domain comprises an amino acid sequence at least 80% identical to SEQ ID NO:14. In some embodiments, the costimulatory domain comprises an amino acid sequence at least 85% identical to SEQ ID NO:14. In some embodiments, the costimulatory domain comprises an amino acid sequence at least 90% identical to SEQ ID NO:14. In some embodiments, the costimulatory domain comprises an amino acid sequence at least 95% identical to SEQ ID NO:14. In some embodiments, the costimulatory domain comprises an amino acid sequence at least 96% identical to SEQ ID NO:14. In some embodiments, the costimulatory domain comprises an amino acid sequence at least 97% identical to SEQ ID NO:14. In some embodiments, the costimulatory domain comprises an amino acid sequence at least 98% identical to SEQ ID NO:14. In some embodiments, the costimulatory domain comprises an amino acid sequence at least 99% identical to SEQ ID NO:14. In some embodiments, the costimulatory domain comprises an amino acid sequence at least 99.5% identical to SEQ ID NO:14. In some embodiments, the costimulatory domain comprises an amino acid sequence at least 99.9% identical to SEQ ID NO:14. In some embodiments, the costimulatory domain comprises an amino acid sequence of SEQ ID NO:14.

In some embodiments, the costimulatory domain comprises an amino acid sequence at least 70%, 75%, 80%, 85%, 90%, 95%, 96%, 97%, 98%, 99%, 99.5%, 99.9%, or 100% identical to SEQ ID NO:15. In some embodiments, the costimulatory domain comprises an amino acid sequence at least 70% identical to SEQ ID NO:15. In some embodiments, the costimulatory domain comprises an amino acid sequence at least 75% identical to SEQ ID NO:15. In some embodiments, the costimulatory domain comprises an amino acid sequence at least 80% identical to SEQ ID NO:15. In some embodiments, the costimulatory domain comprises an amino acid sequence at least 85% identical to SEQ ID NO:15. In some embodiments, the costimulatory domain comprises an amino acid sequence at least 90% identical to SEQ ID NO:15. In some embodiments, the costimulatory domain comprises an amino acid sequence at least 95% identical to SEQ ID NO:15. In some embodiments, the costimulatory domain comprises an amino acid sequence at least 96% identical to SEQ ID NO:15. In some embodiments, the costimulatory domain comprises an amino acid sequence at least 97% identical to SEQ ID NO:15. In some embodiments, the costimulatory domain comprises an amino acid sequence at least 98% identical to SEQ ID NO:15. In some embodiments, the costimulatory domain comprises an amino acid sequence at least 99% identical to SEQ ID NO:15. In some embodiments, the costimulatory domain comprises an amino acid sequence at least 99.5% identical to SEQ ID NO:15. In some embodiments, the costimulatory domain comprises an amino acid sequence at least 99.9% identical to SEQ ID NO:15. In some embodiments, the costimulatory domain comprises an amino acid sequence of SEQ ID NO:15.

IIb. Transmembrane Domain

In various embodiments, the synthetic receptor comprises a transmembrane domain. In some embodiments, the targeting domain and/or the hinge domain are linked to one or more transmembrane domains. In some embodiments, the transmembrane domain comprises one or more positively charged amino acid residues. Exemplary transmembrane domains that can be used in the synthetic receptors disclosed herein are provided in Table 3. In various embodiments, the synthetic receptor comprises a transmembrane domain comprising an amino acid sequence provided in Table 3.

TABLE 3
Exemplary Transmembrane Domains

| Fragment | Sequence |
|---|---|
| KIR2DL4 Transmembrane | AVIRYSVAIILFTILPFFLL |
| domain | (SEQ ID NO: 1) |
| CD8a Transmembrane | YIWAPLAGTCGVLLLSLVITLYC |
| domain | (SEQ ID NO: 2) |
| CD28 Transmembrane | FWVLVVVGGVLACYSLLVTVA |
| domain | FIIFWV (SEQ ID NO: 3) |

In some embodiments, the transmembrane domain comprises or consists of a sequence similar or identical to the sequence of the transmembrane domain of KIR2DL4, or a functional fragment thereof. In some embodiments, the transmembrane domain comprises or consists of a sequence similar or identical to the sequence of the transmembrane domain of CD8a. In some embodiments, the transmembrane domain comprises or consists of a sequence similar or identical to the sequence of the transmembrane domain of CD28.

In some embodiments, the transmembrane domain comprises or consists of a segment having an amino acid sequence at least 70%, 75%, 80%, 85%, 90%, 95%, or 100% sequence identity to SEQ ID NO:1. In some embodiments, the transmembrane domain comprises a segment having an amino acid sequence at least 70% identical to SEQ ID NO:1. In some embodiments, the transmembrane domain comprises a segment having an amino acid sequence at least 75% identical to SEQ ID NO:1. In some embodiments, the transmembrane domain comprises a segment having an amino acid sequence at least 80% identical to SEQ ID NO:1. In some embodiments, the transmembrane domain comprises a segment having an amino acid sequence at least 85% identical to SEQ ID NO:1. In some embodiments, the transmembrane domain comprises a segment having an amino acid sequence at least 90% identical to SEQ ID NO:1. In some embodiments, the transmembrane domain comprises a segment having an amino acid sequence at least 95% identical to SEQ ID NO:1. In some embodiments, the transmembrane domain comprises a segment having an amino acid sequence of SEQ ID NO:1.

In some embodiments, the transmembrane domain comprises or consists of a segment having an amino acid sequence at least 70%, 75%, 80%, 85%, 90%, 95%, or 100% sequence identity to SEQ ID NO:2. In some embodiments, the transmembrane domain comprises a segment having an amino acid sequence at least 70% identical to SEQ ID NO:2. In some embodiments, the transmembrane domain comprises a segment having an amino acid sequence at least 75% identical to SEQ ID NO:2. In some embodiments, the transmembrane domain comprises a segment having an amino acid sequence at least 80% identical to SEQ ID NO:2. In some embodiments, the transmembrane domain comprises a segment having an amino acid sequence at least 85% identical to SEQ ID NO:2. In some embodiments, the transmembrane domain comprises a segment having an amino acid sequence at least 90% identical to SEQ ID NO:2. In some embodiments, the transmembrane domain comprises a segment having an amino acid sequence at least 95% identical to SEQ ID NO:2. In some embodiments, the transmembrane domain comprises a segment having an amino acid sequence of SEQ ID NO:2.

In some embodiments, the transmembrane domain comprises or consists of a segment having an amino acid sequence at least 70%, 75%, 80%, 85%, 90%, 95%, or 100% sequence identity to SEQ ID NO:3. In some embodiments, the transmembrane domain comprises a segment having an amino acid sequence at least 70% identical to SEQ ID NO:3. In some embodiments, the transmembrane domain comprises a segment having an amino acid sequence at least 75% identical to SEQ ID NO:3. In some embodiments, the transmembrane domain comprises a segment having an amino acid sequence at least 80% identical to SEQ ID NO:3. In some embodiments, the transmembrane domain comprises a segment having an amino acid sequence at least 85% identical to SEQ ID NO:3. In some embodiments, the transmembrane domain comprises a segment having an amino acid sequence at least 90% identical to SEQ ID NO:3. In some embodiments, the transmembrane domain comprises a segment having an amino acid sequence at least 95% identical to SEQ ID NO:3. In some embodiments, the transmembrane domain comprises a segment having an amino acid sequence of SEQ ID NO:3.

In some embodiments, the recombinant receptor, such as a CAR, further includes a spacer, which may be or include at least a portion of an immunoglobulin constant region or variant or modified version thereof, such as a hinge region, e.g., an IgG4 hinge region, and/or a CH1/CL and/or Fc region. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, international patent application publication number WO2014/031687, U.S. Pat. No. 8,822,647 or published app. No. US2014/0271635.

IIc. Targeting Domain

In various embodiments, the synthetic receptor comprises a targeting domain. The targeting domain can bind a ligand of interest and induce a conformational change that results in receptor activation. In some embodiments, the targeting domain is an antigen-binding domain. An antigen-binding domain can bind extracellular antigens with high specificity. In some embodiments, the antigen-binding domain comprises an antibody or functional fragment thereof. The term “antigen-binding domain” as used herein is used in the broadest sense and includes polyclonal and monoclonal antibodies, including intact antibodies and functional (antigen-binding) antibody fragments, as well as other moieties that can specifically bind a target antigen.

In various embodiments, the antigen-binding domain is derived from an immunoglobulin. In some embodiments, the antigen-binding domain comprises a heavy chain variable domain (VH) and a light chain variable domain (VL). In some embodiments the antigen binding site comprises a single domain (a single-domain antibody or sdAb), e.g., an antigen binding site derived from a camelid antibody, an antigen binding site derived from a camelid antibody (V_{NAR}), or a nanobody (V_{H}H). Antigen-binding domains can be in any suitable format, including antigen binding fragments (Fab), F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. Antigen-binding domains can also include genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv.

In various embodiments, the antigen binding site comprises a set of complementarity determining regions (CDRs). The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme).

The boundaries of a given CDR may vary depending on the scheme used for identification. For example, the Kabat scheme is based structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

In various embodiments, the antigen-binding domain is not derived from an immunoglobulin. Non-limiting examples of non-immunoglobulin antigen-binding domains include affibodies, affilins, affimers, affitins, alphabodies, anticalins, avimers, peptide aptamers, designed ankyrin repeat proteins (DARPins), monobodys, nanoCLAMPs, endogenous ligands or fragments thereof, small molecules, sugars, lipids, peptides, and any other suitable moiety that specifically and/or selectively binds a target antigen of interest.

The present invention can comprise any suitable antigen-binding domain that currently exists or may be developed in the future.

A non-limiting list of exemplary antigen-binding domains that are suitable for the synthetic receptors disclosed herein is provided as SEQ ID NOs: 35-277. Table 4 identifies the target antigens bound by each antigen-binding domain provided in SEQ ID NOs: 35-277. In various embodiments, the synthetic receptor comprises an antigen-binding domain comprising an amino acid sequence provided in Table 4.

TABLE 4
Antigens Bound by Exemplary Antigen-Binding Domains

| Sequence | Antigen |
|---|---|
| SEQ ID NOs: 53-95 | BCMA |
| SEQ ID NOs: 96-118 | CD19 |
| SEQ ID NOs: 119-156 | CD22 |
| SEQ ID NOs: 157-181 | CD33 |
| SEQ ID NOs: 182-201 | CLL1 |
| SEQ ID NOs: 202-245 | Mesothelin |
| SEQ ID NOs: 246-259 | CD123 |
| SEQ ID NOs: 260-267 | EGFRvIII |
| SEQ ID NOs: 268-270 | EGFR |
| SEQ ID NOs: 271-277 | WT1 ESK |

In some embodiments, the antigen-binding domain comprises an amino acid sequence at least 70%, at least 75%, at least 80%, at least 85%, at least 90%, at least 95%, at least 96%, at least 97%, at least 98%, at least 99%, at least 99.5%, at least 99.9%, or 100% identical to an amino acid sequence selected from any one of SEQ ID NOs: 35-277. In some embodiments, the antigen-binding domain comprises an amino acid sequence at least 70% identical to an amino acid sequence selected from any one of SEQ ID NOs: 35-277. In some embodiments, the antigen-binding domain comprises an amino acid sequence at least 75% identical to an amino acid sequence selected from any one of SEQ ID NOs: 35-277. In some embodiments, the antigen-binding domain comprises an amino acid sequence at least 80% identical to an amino acid sequence selected from any one of SEQ ID NOs: 35-277. In some embodiments, the antigen-binding domain comprises an amino acid sequence at least 85% identical to an amino acid sequence selected from any one of SEQ ID NOs: 35-277. In some embodiments, the antigen-binding domain comprises an amino acid sequence at least 90% identical to an amino acid sequence selected from any one of SEQ ID NOs: 35-277. In some embodiments, the antigen-binding domain comprises an amino acid sequence at least 95% identical to an amino acid sequence selected from any one of SEQ ID NOs: 35-277. In some embodiments, the antigen-binding domain comprises an amino acid sequence at least 99% identical to an amino acid sequence selected from any one of SEQ ID NOs: 35-277. In some embodiments, the antigen-binding domain comprises an amino acid sequence at least 99.5% identical to an amino acid sequence selected from any one of SEQ ID NOs: 35-277. In some embodiments, the antigen-binding domain comprises an amino acid sequence at least 99.9% identical to an amino acid sequence selected from any one of SEQ ID NOs: 35-277. In some embodiments, the antigen-binding domain comprises an amino acid sequence selected from any one of SEQ ID NOs: 35-277.

In some embodiments, the targeting domain binds a regulatory domain of another protein within the cell or on the cell surface. In some embodiments, the targeting domain is intracellular and binds the intracellular domain of a receptor.

IId. Hinge Domain

In various embodiments, the synthetic receptor comprises a hinge domain. A hinge domain can link the extracellular portion (e.g., the targeting domain) of the synthetic receptor to a transmembrane and intracellular portions. The hinge domain can influence activity of the synthetic receptor in addition to its structural role within the protein. Exemplary hinge domains that can be used in the synthetic receptors disclosed herein are provided in Table 5. In various embodiments, the synthetic receptor comprises a hinge domain comprising an amino acid sequence provided in Table 5.

TABLE 5
Exemplary Hinge Domain Sequences

| Fragment | Sequence |
|---|---|
| CD8a Hinge domain | TTTPAPRPPTPAPTIASQPLSLR |
| | PEACRPAAGGAVHTRGLDFACDI |
| | (SEQ ID NO: 4) |
| CD28 Hinge domain | IEVMYPPPYLDNEKSNGTIIHVK |
| | GKHLCPSPLFPGPSKP |
| | (SEQ ID NO: 5) |

In some embodiments, the hinge domain comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NOs: 4 or 5. In some embodiments, the hinge domain comprises an amino acid sequence at least 70% identical to an amino acid sequence of SEQ ID NOs: 4 or 5. In some embodiments, the hinge domain comprises an amino acid sequence at least 75% identical to an amino acid sequence of SEQ ID NOs: 4 or 5. In some embodiments, the hinge domain comprises an amino acid sequence at least 80% identical to an amino acid sequence of SEQ ID NOs: 4 or 5. In some embodiments, the hinge domain comprises an amino acid sequence at least 85% identical to an amino acid sequence of SEQ ID NOs: 4 or 5. In some embodiments, the hinge domain comprises an amino acid sequence at least 90% identical to an amino acid sequence of SEQ ID NOs: 4 or 5. In some embodiments, the hinge domain comprises an amino acid sequence at least 95% identical to an amino acid sequence of SEQ ID NOs: 4 or 5. In some embodiments, the hinge domain comprises an amino acid sequence at least 99% identical to an amino acid sequence of SEQ ID NOs: 4 or 5. In some embodiments, the hinge domain comprises an amino acid sequence of SEQ ID NOs: 4 or 5.

In some embodiments, the hinge domain comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:4. In some embodiments, the hinge domain comprises an amino acid sequence at least 70% identical to an amino acid sequence of SEQ ID NO:4. In some embodiments, the hinge domain comprises an amino acid sequence at least 75% identical to an amino acid sequence of SEQ ID NO:4. In some embodiments, the hinge domain comprises an amino acid sequence at least 80% identical to an amino acid sequence of SEQ ID NO:4. In some embodiments, the hinge domain comprises an amino acid sequence at least 85% identical to an amino acid sequence of SEQ ID NO:4. In some embodiments, the hinge domain comprises an amino acid sequence at least 90% identical to an amino acid sequence of SEQ ID NO:4. In some embodiments, the hinge domain comprises an amino acid sequence at least 95% identical to an amino acid sequence of SEQ ID NO:4. In some embodiments, the hinge domain comprises an amino acid sequence at least 99% identical to an amino acid sequence of SEQ ID NO:4. In some embodiments, the hinge domain comprises an amino acid sequence of SEQ ID NO:4.

In some embodiments, the hinge domain comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:5. In some embodiments, the hinge domain comprises an amino acid sequence at least 70% identical to an amino acid sequence of SEQ ID NO:5. In some embodiments, the hinge domain comprises an amino acid sequence at least 75% identical to an amino acid sequence of SEQ ID NO:5. In some embodiments, the hinge domain comprises an amino acid sequence at least 80% identical to an amino acid sequence of SEQ ID NO:5. In some embodiments, the hinge domain comprises an amino acid sequence at least 85% identical to an amino acid sequence of SEQ ID NO:5. In some embodiments, the hinge domain comprises an amino acid sequence at least 90% identical to an amino acid sequence of SEQ ID NO:5. In some embodiments, the hinge domain comprises an amino acid sequence at least 95% identical to an amino acid sequence of SEQ ID NO:5. In some embodiments, the hinge domain comprises an amino acid sequence at least 99% identical to an amino acid sequence of SEQ ID NO:5. In some embodiments, the hinge domain comprises an amino acid sequence of SEQ ID NO:5.

IIe. Full-Length Receptors and Receptor Fragments

Table 6 shows exemplary sequences of fragments of synthetic receptors, which can be operably linked to any targeting domain described herein or otherwise known in the art. In some embodiments, a synthetic receptor comprises, from N-terminus to C-terminus, a targeting domain, a hinge, a transmembrane domain (TM), and an intracellular signaling domain (ICD). In various embodiments, the synthetic receptor comprises an amino acid sequence provided in Table 6.

TABLE 6
Exemplary Fragments of Synthetic Receptors

| Fragment | Sequence |
|---|---|
| KIR2DL4 | AVIRYSVAIILFTILPFFLL |
| transmembrane-ICD | HRWCSKKKDAAVMNQEPAGH |
| | RTVNREDSDEQDPQEVTYAQ |
| | LDHCIFTQRKITGPSQRSKR |
| | PSTDTSVCIELPNAEPRALS |
| | PAHEHHSQALMGSSRETTAL |
| | SQTQLASSNVPAAGI |
| | (SEQ ID NO: 17) |
| KIR2DL4 TM-ICD + | AVIRYSVAIILFTILPFFLL |
| CD3z | HRWCSKKKDAAVMNQEPAGH |
| | RTVNREDSDEQDPQEVTYAQ |
| | LDHCIFTQRKITGPSQRSKR |
| | PSTDTSVCIELPNAEPRALS |
| | PAHEHHSQALMGSSRETTAL |
| | SQTQLASSNVPAAGIRVKFS |
| | RSADAPAYKQGQNQLYNELN |
| | LGRREEYDVLDKRRGRDPEM |
| | GGKPRRKNPQEGLYNELQKD |
| | KMAEAYSEIGMKGERRRGKG |
| | HDGLYQGLSTATKDTYDALH |
| | MQALPPR |
| | (SEQ ID NO: 18) |
| KIR2DL4 TM-ICD | AVIRYSVAIILFTILPFFLL |
| Truncation A | HRWCSKKKDAAVMNQEPAGH |
| | RTVNREDSD |
| | (SEQ ID NO: 19) |
| KIR2DL4 TM-ICD | AVIRYSVAIILFTILPFFLL |
| Truncation A + | HRWCSKKKDAAVMNQEPAGH |
| CD3z | RTVNREDSDRVKFSRSADAP |
| | AYKQGQNQLYNELNLGRREE |
| | YDVLDKRRGRDPEMGGKPRR |
| | KNPQEGLYNELQKDKMAEAY |
| | SEIGMKGERRRGKGHDGLYQ |
| | GLSTATKDTYDALHMQALPP |
| | R |
| | (SEQID NO: 20) |
| KIR2DL4 TM-ICD | AVIRYSVAIILFTILPFFLL |
| ITIM mut | HRWCSKKKDAAVMNQEPAGH |
| | RTVNREDSDEQDPQEVAFAQ |
| | LDHCIFTQRKITGPSQRSKR |
| | PSTDTSVCIELPNAEPRALS |
| | PAHEHHSQALMGSSRETTAL |
| | SQTQLASSNVPAAGI |
| | (SEQ ID NO: 21) |
| KIR2DL4 TM-ICD | AVIRYSVAIILFTILPFFLL |
| ITIM mut + CD3z | HRWCSKKKDAAVMNQEPAGH |
| | RTVNREDSDEQDPQEVAFAQ |
| | LDHCIFTQRKITGPSQRSKR |
| | PSTDTSVCIELPNAEPRALS |
| | PAHEHHSQALMGSSRETTAL |
| | SQTQLASSNVPAAGIRVKFS |
| | RSADAPAYKQGQNQLYNELN |
| | LGRREEYDVLDKRRGRDPEM |
| | GGKPRRKNPQEGLYNELQKD |
| | KMAEAYSEIGMKGERRRGKG |
| | HDGLYQGLSTATKDTYDALH |
| | MQALPPR |
| | (SEQ ID NO: 22) |
| KIR2DL4 TM-ICD | AVIRYSVAIILFTILPFFLL |
| Truncation B ITIM | HRWCSKKKDAAVMNQEPAGH |
| mut | RTVNREDSDEQDPQEVAFAQ |
| | LDHCIFTQR |
| | (SEQ ID NO: 23) |
| KIR2DL4 TM-ICD | AVIRYSVAIILFTILPFFLL |
| Truncation B ITIM | HRWCSKKKDAAVMNQEPAGH |
| mut +CD3z | RTVNREDSDEQDPQEVAFAQ |
| | LDHCIFTQRRVKFSRSADAP |
| | AYKQGQNQLYNELNLGRREE |
| | YDVLDKRRGRDPEMGGKPRR |
| | KNPQEGLYNELQKDKMAEAY |
| | SEIGMKGERRRGKGHDGLYQ |
| | GLSTATKDTYDALHMQALPP |
| | R |
| | (SEQ ID NO: 24) |
| KIR2DL4 TM-ICD | AVIRYSVAIILFTILPFFLL |
| Truncation C ITIM | HRWCSKKKDAAVMNQEPAGH |
| mut | RTVNREDSDEQDPQEVAFAQ |
| | LDHCIFTQRKITGPSQRS |
| | (SEQ ID NO: 25) |
| KIR2DL4 TM-ICD | AVIRYSVAIILFTILPFFLL |
| Truncation C ITIM | HRWCSKKKDAAVMNQEPAGH |
| mut + CD3z | RTVNREDSDEQDPQEVAFAQ |
| | LDHCIFTQRKITGPSQRSRV |
| | KFSRSADAPAYKQGQNQLYN |
| | ELNLGRREEYDVLDKRRGRD |
| | PEMGGKPRRKNPQEGLYNEL |
| | QKDKMAEAYSEIGMKGERRR |
| | GKGHDGLYQGLSTATKDTYD |
| | ALHMQALPPR |
| | (SEQ ID NO: 26) |
| CD8a TM-CD3z | YIWAPLAGTCGVLLLSLVIT |
| | LYCRVKFSRSADAPAYKQGQ |
| | NQLYNELNLGRREEYDVLDK |
| | RRGRDPEMGGKPRRKNPQEG |
| | LYNELQKDKMAEAYSEIGMK |
| | GERRRGKGHDGLYQGLSTAT |
| | KDTYDALHMQALPPR |
| | (SEQ ID NO: 27) |
| CD8a TM-KIR2DL4 | YIWAPLAGTCGVLLLSLVIT |
| ICD | LYCHRWCSKKKDAAVMNQEP |
| | AGHRTVNREDSDEQDPQEVA |
| | FAQLDHCIFTQRKITGPSQR |
| | SKRPSTDTSVCIELPNAEPR |
| | ALSPAHEHHSQALMGSSRET |
| | TALSQTQLASSNVPAAGI |
| | (SED ID NO: 28) |
| CD28 TM-41BB | FWVLVVVGGVLACYSLLVTV |
| ICD-CD3z | AFIIFWVKRGRKKLLYIFKQ |
| | PFMRPVQTTQEEDGCSCRFP |
| | EEEEGGCELRVKFSRSADAP |
| | AYQQGQNQLYNELNLGRREE |
| | YDVLDKRRGRDPEMGGKPRR |
| | KNPQEGLYNELQKDKMAEAY |
| | SEIGMKGERRRGKGHDGLYQ |
| | GLSTATKDTYDALHMQALPP |
| | R |
| | (SEQ ID NO: 29) |
| CD28 hinge-CD28 | IEVMYPPPYLDNEKSNGTII |
| TM-CD28 ICD- | HVKGKHLCPSPLFPGPSKPF |
| CD3z | WVLVVVGGVLACYSLLVTVA |
| | FIIFWVRSKRSRLLHSDYMN |
| | MTPRRPGPTRKHYQPYAPPR |
| | DFAAYRSRVKFSRSADAPAY |
| | QQGQNQLYNELNLGRREEYD |
| | VLDKRRGRDPEMGGKPRRKN |
| | PQEGLYNELQKDKMAEAYSE |
| | IGMKGERRRGKGHDGLYQGL |
| | STATKDTYDALHMQALPPR |
| | (SEQ ID NO: 30) |
| CD8a TM-4-1BB | YIWAPLAGTCGVLLLSLVIT |
| ICD-CD3z | LYCKRGRKKLLYIFKQPFMR |
| | PVQTTQEEDGCSCRFPEEEE |
| | GGCELRVKFSRSADAPAYQQ |
| | GQNQLYNELNLGRREEYDVL |
| | DKRRGRDPEMGGKPRRKNPQ |
| | EGLYNELQKDKMAEAYSEIG |
| | MKGERRRGKGHDGLYQGLST |
| | ATKDTYDALHMQALPPR |
| | (SEQ ID NO: 31) |
| KIR2DL4 TM- | AVIRYSVAIILFTILPFFLL |
| KIR2DL4(C8) ICD1 | HRWCSKKKDAAVMNQEPAGH |
| | RTVNREDSDEQDPQEVTYAQ |
| | LDHCIFTQR |
| | (SEQ ID NO: 32) |
| KIR2DL4 TM- | AVIRYSVAIILFTILPFFLL |
| KIR2DL4(C8) ICD1- | HRWCSKKKDAAVMNQEPAGH |
| 41BB ICD2 | RTVNREDSDEQDPQEVTYAQ |
| | LDHCIFTQRKRGRKKLLYIF |
| | KQPFMRPVQTTQEEDGCSCR |
| | FPEEEEGGCEL |
| | (SEQ ID NO: 33) |
| KIR2DL4 TM- | AVIRYSVAIILFTILPFFLL |
| KIR2DL4(C8) ICD1- | HRWCSKKKDAAVMNQEPAGH |
| DAP10 ICD2 | RTVNREDSDEQDPQEVTYAQ |
| | LDHCIFTQRLCARPRRSPAQ |
| | EDGKVYINMPGRG |
| | (SEQ ID NO: 34) |

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence selected from any one of SEQ ID NOs:17-34. In some embodiments, the synthetic receptor comprises an amino acid sequence at least 70% identical to an amino acid sequence selected from any one of SEQ ID NOs:17-34. In some embodiments, the synthetic receptor comprises an amino acid sequence at least 75% identical to an amino acid sequence selected from any one of SEQ ID NOs:17-34. In some embodiments, the synthetic receptor comprises an amino acid sequence at least 80% identical to an amino acid sequence selected from any one of SEQ ID NOs:17-34. In some embodiments, the synthetic receptor comprises an amino acid sequence at least 85% identical to an amino acid sequence selected from any one of SEQ ID NOs:17-34. In some embodiments, the synthetic receptor comprises an amino acid sequence at least 90% identical to an amino acid sequence selected from any one of SEQ ID NOs:17-34. In some embodiments, the synthetic receptor comprises an amino acid sequence at least 95% identical to an amino acid sequence selected from any one of SEQ ID NOs:17-34. In some embodiments, the synthetic receptor comprises an amino acid sequence at least 99% identical to an amino acid sequence selected from any one of SEQ ID NOs:17-34. In some embodiments, the synthetic receptor comprises an amino acid sequence selected from any one of SEQ ID NOs:17-34.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:17. In some embodiments, the synthetic receptor comprises an amino acid sequence at least 70% identical to an amino acid sequence of SEQ ID NO:17. In some embodiments, the synthetic receptor comprises an amino acid sequence at least 75% identical to an amino acid sequence of SEQ ID NO:17. In some embodiments, the synthetic receptor comprises an amino acid sequence at least 80% identical to an amino acid sequence of SEQ ID NO:17. In some embodiments, the synthetic receptor comprises an amino acid sequence at least 85% identical to an amino acid sequence of SEQ ID NO:17. In some embodiments, the synthetic receptor comprises an amino acid sequence at least 90% identical to an amino acid sequence of SEQ ID NO:17. In some embodiments, the synthetic receptor comprises an amino acid sequence at least 95% identical to an amino acid sequence of SEQ ID NO:17. In some embodiments, the synthetic receptor comprises an amino acid sequence at least 99% identical to an amino acid sequence of SEQ ID NO:17. In some embodiments, the synthetic receptor comprises an amino acid sequence of SEQ ID NO:17.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:18. In some embodiments, the synthetic receptor comprises an amino acid sequence at least 70% identical to an amino acid sequence of SEQ ID NO:18. In some embodiments, the synthetic receptor comprises an amino acid sequence at least 75% identical to an amino acid sequence of SEQ ID NO:18. In some embodiments, the synthetic receptor comprises an amino acid sequence at least 80% identical to an amino acid sequence of SEQ ID NO:18. In some embodiments, the synthetic receptor comprises an amino acid sequence at least 85% identical to an amino acid sequence of SEQ ID NO:18. In some embodiments, the synthetic receptor comprises an amino acid sequence at least 90% identical to an amino acid sequence of SEQ ID NO:18. In some embodiments, the synthetic receptor comprises an amino acid sequence at least 95% identical to an amino acid sequence of SEQ ID NO:18. In some embodiments, the synthetic receptor comprises an amino acid sequence at least 99% identical to an amino acid sequence of SEQ ID NO:18. In some embodiments, the synthetic receptor comprises an amino acid sequence of SEQ ID NO:18.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:19. In some embodiments, the synthetic receptor comprises an amino acid sequence at least 70% identical to an amino acid sequence of SEQ ID NO:19. In some embodiments, the synthetic receptor comprises an amino acid sequence at least 75% identical to an amino acid sequence of SEQ ID NO:19. In some embodiments, the synthetic receptor comprises an amino acid sequence at least 80% identical to an amino acid sequence of SEQ ID NO:19. In some embodiments, the synthetic receptor comprises an amino acid sequence at least 85% identical to an amino acid sequence of SEQ ID NO:19. In some embodiments, the synthetic receptor comprises an amino acid sequence at least 90% identical to an amino acid sequence of SEQ ID NO:19. In some embodiments, the synthetic receptor comprises an amino acid sequence at least 95% identical to an amino acid sequence of SEQ ID NO:19. In some embodiments, the synthetic receptor comprises an amino acid sequence at least 99% identical to an amino acid sequence of SEQ ID NO:19. In some embodiments, the synthetic receptor comprises an amino acid sequence of SEQ ID NO:19.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:20. In some embodiments, the synthetic receptor comprises an amino acid sequence at least 70% identical to an amino acid sequence of SEQ ID NO:20. In some embodiments, the synthetic receptor comprises an amino acid sequence at least 75% identical to an amino acid sequence of SEQ ID NO:20. In some embodiments, the synthetic receptor comprises an amino acid sequence at least 80% identical to an amino acid sequence of SEQ ID NO:20. In some embodiments, the synthetic receptor comprises an amino acid sequence at least 85% identical to an amino acid sequence of SEQ ID NO:20. In some embodiments, the synthetic receptor comprises an amino acid sequence at least 90% identical to an amino acid sequence of SEQ ID NO:20. In some embodiments, the synthetic receptor comprises an amino acid sequence at least 95% identical to an amino acid sequence of SEQ ID NO:20. In some embodiments, the synthetic receptor comprises an amino acid sequence at least 99% identical to an amino acid sequence of SEQ ID NO:20. In some embodiments, the synthetic receptor comprises an amino acid sequence of SEQ ID NO:20.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:21. In some embodiments, the synthetic receptor comprises an amino acid sequence at least 70% identical to an amino acid sequence of SEQ ID NO:21. In some embodiments, the synthetic receptor comprises an amino acid sequence at least 75% identical to an amino acid sequence of SEQ ID NO:21. In some embodiments, the synthetic receptor comprises an amino acid sequence at least 80% identical to an amino acid sequence of SEQ ID NO:21. In some embodiments, the synthetic receptor comprises an amino acid sequence at least 85% identical to an amino acid sequence of SEQ ID NO:21. In some embodiments, the synthetic receptor comprises an amino acid sequence at least 90% identical to an amino acid sequence of SEQ ID NO:21. In some embodiments, the synthetic receptor comprises an amino acid sequence at least 95% identical to an amino acid sequence of SEQ ID NO:21. In some embodiments, the synthetic receptor comprises an amino acid sequence at least 99% identical to an amino acid sequence of SEQ ID NO:21. In some embodiments, the synthetic receptor comprises an amino acid sequence of SEQ ID NO:21.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:22. In some embodiments, the synthetic receptor comprises an amino acid sequence at least 70% identical to an amino acid sequence of SEQ ID NO:22. In some embodiments, the synthetic receptor comprises an amino acid sequence at least 75% identical to an amino acid sequence of SEQ ID NO:22. In some embodiments, the synthetic receptor comprises an amino acid sequence at least 80% identical to an amino acid sequence of SEQ ID NO:22. In some embodiments, the synthetic receptor comprises an amino acid sequence at least 85% identical to an amino acid sequence of SEQ ID NO:22. In some embodiments, the synthetic receptor comprises an amino acid sequence at least 90% identical to an amino acid sequence of SEQ ID NO:22. In some embodiments, the synthetic receptor comprises an amino acid sequence at least 95% identical to an amino acid sequence of SEQ ID NO:22. In some embodiments, the synthetic receptor comprises an amino acid sequence at least 99% identical to an amino acid sequence of SEQ ID NO:22. In some embodiments, the synthetic receptor comprises an amino acid sequence of SEQ ID NO:22.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:23. In some embodiments, the synthetic receptor comprises an amino acid sequence at least 70% identical to an amino acid sequence of SEQ ID NO:23. In some embodiments, the synthetic receptor comprises an amino acid sequence at least 75% identical to an amino acid sequence of SEQ ID NO:23. In some embodiments, the synthetic receptor comprises an amino acid sequence at least 80% identical to an amino acid sequence of SEQ ID NO:23. In some embodiments, the synthetic receptor comprises an amino acid sequence at least 85% identical to an amino acid sequence of SEQ ID NO:23. In some embodiments, the synthetic receptor comprises an amino acid sequence at least 90% identical to an amino acid sequence of SEQ ID NO:23. In some embodiments, the synthetic receptor comprises an amino acid sequence at least 95% identical to an amino acid sequence of SEQ ID NO:23. In some embodiments, the synthetic receptor comprises an amino acid sequence at least 99% identical to an amino acid sequence of SEQ ID NO:23. In some embodiments, the synthetic receptor comprises an amino acid sequence of SEQ ID NO:23.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:24. In some embodiments, the synthetic receptor comprises an amino acid sequence at least 70% identical to an amino acid sequence of SEQ ID NO:24. In some embodiments, the synthetic receptor comprises an amino acid sequence at least 75% identical to an amino acid sequence of SEQ ID NO:24. In some embodiments, the synthetic receptor comprises an amino acid sequence at least 80% identical to an amino acid sequence of SEQ ID NO:24. In some embodiments, the synthetic receptor comprises an amino acid sequence at least 85% identical to an amino acid sequence of SEQ ID NO:24. In some embodiments, the synthetic receptor comprises an amino acid sequence at least 90% identical to an amino acid sequence of SEQ ID NO:24. In some embodiments, the synthetic receptor comprises an amino acid sequence at least 95% identical to an amino acid sequence of SEQ ID NO:24. In some embodiments, the synthetic receptor comprises an amino acid sequence at least 99% identical to an amino acid sequence of SEQ ID NO:24. In some embodiments, the synthetic receptor comprises an amino acid sequence of SEQ ID NO:24.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:25. In some embodiments, the synthetic receptor comprises an amino acid sequence at least 70% identical to an amino acid sequence of SEQ ID NO:25. In some embodiments, the synthetic receptor comprises an amino acid sequence at least 75% identical to an amino acid sequence of SEQ ID NO:25. In some embodiments, the synthetic receptor comprises an amino acid sequence at least 80% identical to an amino acid sequence of SEQ ID NO:25. In some embodiments, the synthetic receptor comprises an amino acid sequence at least 85% identical to an amino acid sequence of SEQ ID NO:25. In some embodiments, the synthetic receptor comprises an amino acid sequence at least 90% identical to an amino acid sequence of SEQ ID NO:25. In some embodiments, the synthetic receptor comprises an amino acid sequence at least 95% identical to an amino acid sequence of SEQ ID NO:25. In some embodiments, the synthetic receptor comprises an amino acid sequence at least 99% identical to an amino acid sequence of SEQ ID NO:25. In some embodiments, the synthetic receptor comprises an amino acid sequence of SEQ ID NO:25.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:26. In some embodiments, the synthetic receptor comprises an amino acid sequence at least 70% identical to an amino acid sequence of SEQ ID NO:26. In some embodiments, the synthetic receptor comprises an amino acid sequence at least 75% identical to an amino acid sequence of SEQ ID NO:26. In some embodiments, the synthetic receptor comprises an amino acid sequence at least 80% identical to an amino acid sequence of SEQ ID NO:26. In some embodiments, the synthetic receptor comprises an amino acid sequence at least 85% identical to an amino acid sequence of SEQ ID NO:26. In some embodiments, the synthetic receptor comprises an amino acid sequence at least 90% identical to an amino acid sequence of SEQ ID NO:26. In some embodiments, the synthetic receptor comprises an amino acid sequence at least 95% identical to an amino acid sequence of SEQ ID NO:26. In some embodiments, the synthetic receptor comprises an amino acid sequence at least 99% identical to an amino acid sequence of SEQ ID NO:26. In some embodiments, the synthetic receptor comprises an amino acid sequence of SEQ ID NO:26.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:27. In some embodiments, the synthetic receptor comprises an amino acid sequence at least 70% identical to an amino acid sequence of SEQ ID NO:27. In some embodiments, the synthetic receptor comprises an amino acid sequence at least 75% identical to an amino acid sequence of SEQ ID NO:27. In some embodiments, the synthetic receptor comprises an amino acid sequence at least 80% identical to an amino acid sequence of SEQ ID NO:27. In some embodiments, the synthetic receptor comprises an amino acid sequence at least 85% identical to an amino acid sequence of SEQ ID NO:27. In some embodiments, the synthetic receptor comprises an amino acid sequence at least 90% identical to an amino acid sequence of SEQ ID NO:27. In some embodiments, the synthetic receptor comprises an amino acid sequence at least 95% identical to an amino acid sequence of SEQ ID NO:27. In some embodiments, the synthetic receptor comprises an amino acid sequence at least 99% identical to an amino acid sequence of SEQ ID NO:27. In some embodiments, the synthetic receptor comprises an amino acid sequence of SEQ ID NO:27.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:28. In some embodiments, the synthetic receptor comprises an amino acid sequence at least 70% identical to an amino acid sequence of SEQ ID NO:28. In some embodiments, the synthetic receptor comprises an amino acid sequence at least 75% identical to an amino acid sequence of SEQ ID NO:28. In some embodiments, the synthetic receptor comprises an amino acid sequence at least 80% identical to an amino acid sequence of SEQ ID NO:28. In some embodiments, the synthetic receptor comprises an amino acid sequence at least 85% identical to an amino acid sequence of SEQ ID NO:28. In some embodiments, the synthetic receptor comprises an amino acid sequence at least 90% identical to an amino acid sequence of SEQ ID NO:28. In some embodiments, the synthetic receptor comprises an amino acid sequence at least 95% identical to an amino acid sequence of SEQ ID NO:28. In some embodiments, the synthetic receptor comprises an amino acid sequence at least 99% identical to an amino acid sequence of SEQ ID NO:28. In some embodiments, the synthetic receptor comprises an amino acid sequence of SEQ ID NO:28.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:29. In some embodiments, the synthetic receptor comprises an amino acid sequence at least 70% identical to an amino acid sequence of SEQ ID NO:29. In some embodiments, the synthetic receptor comprises an amino acid sequence at least 75% identical to an amino acid sequence of SEQ ID NO:29. In some embodiments, the synthetic receptor comprises an amino acid sequence at least 80% identical to an amino acid sequence of SEQ ID NO:29. In some embodiments, the synthetic receptor comprises an amino acid sequence at least 85% identical to an amino acid sequence of SEQ ID NO:29. In some embodiments, the synthetic receptor comprises an amino acid sequence at least 90% identical to an amino acid sequence of SEQ ID NO:29. In some embodiments, the synthetic receptor comprises an amino acid sequence at least 95% identical to an amino acid sequence of SEQ ID NO:29. In some embodiments, the synthetic receptor comprises an amino acid sequence at least 99% identical to an amino acid sequence of SEQ ID NO:29. In some embodiments, the synthetic receptor comprises an amino acid sequence of SEQ ID NO:29.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:30. In some embodiments, the synthetic receptor comprises an amino acid sequence at least 70% identical to an amino acid sequence of SEQ ID NO:30. In some embodiments, the synthetic receptor comprises an amino acid sequence at least 75% identical to an amino acid sequence of SEQ ID NO:30. In some embodiments, the synthetic receptor comprises an amino acid sequence at least 80% identical to an amino acid sequence of SEQ ID NO:30. In some embodiments, the synthetic receptor comprises an amino acid sequence at least 85% identical to an amino acid sequence of SEQ ID NO:30. In some embodiments, the synthetic receptor comprises an amino acid sequence at least 90% identical to an amino acid sequence of SEQ ID NO:30. In some embodiments, the synthetic receptor comprises an amino acid sequence at least 95% identical to an amino acid sequence of SEQ ID NO:30. In some embodiments, the synthetic receptor comprises an amino acid sequence at least 99% identical to an amino acid sequence of SEQ ID NO:30. In some embodiments, the synthetic receptor comprises an amino acid sequence of SEQ ID NO:30.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:31. In some embodiments, the synthetic receptor comprises an amino acid sequence at least 70% identical to an amino acid sequence of SEQ ID NO:31. In some embodiments, the synthetic receptor comprises an amino acid sequence at least 75% identical to an amino acid sequence of SEQ ID NO:31. In some embodiments, the synthetic receptor comprises an amino acid sequence at least 80% identical to an amino acid sequence of SEQ ID NO:31. In some embodiments, the synthetic receptor comprises an amino acid sequence at least 85% identical to an amino acid sequence of SEQ ID NO:31. In some embodiments, the synthetic receptor comprises an amino acid sequence at least 90% identical to an amino acid sequence of SEQ ID NO:31. In some embodiments, the synthetic receptor comprises an amino acid sequence at least 95% identical to an amino acid sequence of SEQ ID NO:31. In some embodiments, the synthetic receptor comprises an amino acid sequence at least 99% identical to an amino acid sequence of SEQ ID NO:31. In some embodiments, the synthetic receptor comprises an amino acid sequence of SEQ ID NO:31.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:32. In some embodiments, the synthetic receptor comprises an amino acid sequence at least 70% identical to an amino acid sequence of SEQ ID NO:32. In some embodiments, the synthetic receptor comprises an amino acid sequence at least 75% identical to an amino acid sequence of SEQ ID NO:32. In some embodiments, the synthetic receptor comprises an amino acid sequence at least 80% identical to an amino acid sequence of SEQ ID NO:32. In some embodiments, the synthetic receptor comprises an amino acid sequence at least 85% identical to an amino acid sequence of SEQ ID NO:32. In some embodiments, the synthetic receptor comprises an amino acid sequence at least 90% identical to an amino acid sequence of SEQ ID NO:32. In some embodiments, the synthetic receptor comprises an amino acid sequence at least 95% identical to an amino acid sequence of SEQ ID NO:32. In some embodiments, the synthetic receptor comprises an amino acid sequence at least 99% identical to an amino acid sequence of SEQ ID NO:32. In some embodiments, the synthetic receptor comprises an amino acid sequence of SEQ ID NO:32.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:33. In some embodiments, the synthetic receptor comprises an amino acid sequence at least 70% identical to an amino acid sequence of SEQ ID NO:33. In some embodiments, the synthetic receptor comprises an amino acid sequence at least 75% identical to an amino acid sequence of SEQ ID NO:33. In some embodiments, the synthetic receptor comprises an amino acid sequence at least 80% identical to an amino acid sequence of SEQ ID NO:33. In some embodiments, the synthetic receptor comprises an amino acid sequence at least 85% identical to an amino acid sequence of SEQ ID NO:33. In some embodiments, the synthetic receptor comprises an amino acid sequence at least 90% identical to an amino acid sequence of SEQ ID NO:33. In some embodiments, the synthetic receptor comprises an amino acid sequence at least 95% identical to an amino acid sequence of SEQ ID NO:33. In some embodiments, the synthetic receptor comprises an amino acid sequence at least 99% identical to an amino acid sequence of SEQ ID NO:33. In some embodiments, the synthetic receptor comprises an amino acid sequence of SEQ ID NO:33.

In some embodiments, the synthetic receptor comprises an amino acid sequence at least 70%, at least 75%, at least 80%, at least 85%, at least 90%, at least 95%, at least 96%, at least 97%, at least 98%, at least 99%, or 100% identical to an amino acid sequence of SEQ ID NO:34. In some embodiments, the synthetic receptor comprises an amino acid sequence at least 70% identical to an amino acid sequence of SEQ ID NO:34. In some embodiments, the synthetic receptor comprises an amino acid sequence at least 75% identical to an amino acid sequence of SEQ ID NO:34. In some embodiments, the synthetic receptor comprises an amino acid sequence at least 80% identical to an amino acid sequence of SEQ ID NO:34. In some embodiments, the synthetic receptor comprises an amino acid sequence at least 85% identical to an amino acid sequence of SEQ ID NO:34. In some embodiments, the synthetic receptor comprises an amino acid sequence at least 90% identical to an amino acid sequence of SEQ ID NO:34. In some embodiments, the synthetic receptor comprises an amino acid sequence at least 95% identical to an amino acid sequence of SEQ ID NO:34. In some embodiments, the synthetic receptor comprises an amino acid sequence at least 99% identical to an amino acid sequence of SEQ ID NO:34. In some embodiments, the synthetic receptor comprises an amino acid sequence of SEQ ID NO:34.

### III. Recombinant Immune Cells

Disclosed herein, is an active recombinant immune cell expressing a synthetic receptor and a method of activating the same. An “active immune cell” is an immune cell that has encountered an antigen presented by an “antigen-presenting cell” (or “APC”). An APC presents an antigen to an immune cell through major histocompatibility complex (MHC) proteins. The major histocompatibility complex (MHC) is a large genetic complex with multiple loci. The MHC loci encode two major classes of MHC membrane molecules, referred to as class I and class II MHCs. “Professional APCs” (e.g., dendritic cells, mononuclear phagocytes, or B cells) express MHC-class II, whereas non-professional APCs, which can be any nucleated cell that is not a “professional APC,” express MHC-class I. Upon encountering an APC, immune cells enter an “activated state,” wherein the active immune cell can directly induce cell death in target cells or can promote the function of other immune cells (e.g., by releasing cytokines into the extracellular milieu). An active immune cell is contrasted to an “inactive immune cell.” An inactive immune cell does not exert effects on target cells or other immune cells. An inactive immune cell can be a “naive immune cell,” meaning the immune cell has never encountered an antigen. An inactive immune cell can also be a previously active immune cell that was inactivated (e.g., by encountering a “regulatory” immune cell).

IIIa. Types of Immune Cells

### T Cells

In some embodiments, the recombinant immune cell can be a T cell. T cells are typically activated by productive interactions between a T cell receptor (TCR) complex and a major histocompatibility (MHC) antigen complex on the surface of a target cell. In some cases, immune cell activation against a target cell requires co-stimulation. For example, T cell activation can require stimulation through both the interaction between the TCR and MHC antigen complex (Signal 1) and interactions between one or more co-receptors and one or more target cell antigens (Signal 2). Failure to provide Signal 2 may result in T cell anergy/tolerance or apoptosis (Macian et al, Curr Opin Immunol, 16(2):209- 216, 2004; Pardigon et al, J Immunol, 164(9):4493-4499, 2000; Ward, Biochem J, 318 (Pt 2):361-377, 1996; Zhong et al, Mol Ther, 18(2):413-420, 2010).

### CD8⁺ T Cells

In some embodiments, the T cell is a CD8-positive “cytotoxic” T cell. Cytotoxic T cells recognize and kill target cells that display peptide fragments presented on the cell surface in the context of MHC-I molecules. Cytotoxic T cells store preformed cytotoxins in lytic granules which fuse with the membranes of target cells. CD8+ cytotoxic T cells additionally express Fas ligand (FasL), which induces apoptosis in Fas-expressing target cells.

CD4⁺ T cells

In some embodiments, the T cell is a CD4-positive “T helper” (T_{H}) cell. T_{H} cells function to regulate the proliferation of B cells and B cell responses. T_{H} cells play an importance role in humoral immunity and immunopathology. CD4⁺ T helper cells differentiate into either T_{H}1 and T_{H}2 cells, both of which express CD4 and recognize peptide fragments processed within intracellular vesicles and presented on the cell surface in the context of MHC-II molecules. T_{H}1 cells can directly or indirectly activate a number of other immune cells, including macrophages and B cells, thereby promoting more efficient destruction and clearance of intracellular microorganisms. T_{H}1 cells can also be involved in pathways that lead to activation of CD8 cytotoxic T cells (e.g., dendritic cell “licensing”). T_{H}2 cells stimulate the differentiation of B cells and promote the production of antibodies and other effector molecules of the humoral immune response. T_{H} cells can differentiate into T_{H}1 or T_{H}2 T cells depending upon antigen stimulation and cytokine environment. T helper cells first activated by antigen in the presence of IL-12 develop predominantly into T_{H}1 cells, whereas those activated in the presence of IL-4 develop predominantly into T_{H}2 cells. Progenitor T helper cells may require individual cellular divisions before becoming competent to synthesize the cytokines that are indicative of either the T_{H}1 or T_{H}2 pathway. T_{H}1 and T_{H}2 cell phenotypes are different from each other in early activation signal transduction pathways, especially in the role of TCR related protein tyrosine kinases. TCR and its downstream protein tyrosine kinases such as Fyn, p56(Ick), and ZAP-70 are involved in the development and differentiation of T_{H}1 and T_{H}2 cells.

T_{H}1 and T_{H}2 cells both express CD4 and recognize peptide fragments processed within intracellular vesicles and presented on the cell surface in the context of MHC-II molecules. T_{H}1 cells can activate a number of other immune cells, including macrophages and B cells, thereby promoting more efficient destruction and clearance of intracellular microorganisms. T_{H}1 cells can also be involved in pathways that lead to activation of CD8⁺ cytotoxic T cells (e.g., dendritic cell “licensing”). T_{H}2 cells stimulate the differentiation of B cells and promote the production of antibodies and other effector molecules of the humoral immune response.

T_{H}17 cells are a subset of pro-inflammatory T_{H} cells that express IL-17. T_{H}17 cells are developmentally distinct from T_{H}1 and T_{H}2 cells. The signaling pathway that induces differentiation of T_{H} cells into T_{H}17 cells inhibits T_{reg} differentiation.

T follicular helper cells (T_{FH}) are a subset of CD4⁺ cells. T follicular helper cells are essential for helping cognate B cells form and maintain the germinal center (GC) reaction, and for development of humoral immune responses. These cells are universally defined by expression of the chemokine receptor CXCR5, which directs them to the B cell follicles via gradients of the chemokine CXCL131. T_{FH} cells also express the transcription factor Bcl6 (which represses Blimp-1/Prdm1) and high levels of the costimulatory receptor ICOS, which are both critical for their differentiation and maintenance. In addition, T_{FH} cells secrete large amounts of IL-21, which aids in GC formation, isotype switching and plasma cell formation. In humans and mice functionally similar T_{FH} cells can be found in secondary lymphoid organs. CXCR5+T_{FH} cells are also present in peripheral blood and seen at elevated levels in individuals with autoantibodies.

### NK-T Cells

In In some embodiments, the T cell is a natural killer T (“NK-T”) cell. Natural killer T cells represent a subset of T lymphocytes with unique properties. NK-T cells are different from functionally differentiated conventional αβ T cells in that they share properties of both natural killer cells and T cells are can rapidly produce both T_{H}1-type and T_{H}2-type responses upon stimulation with their ligands (innate immunity). The activation of NK-T cells paradoxically can lead either to suppression or stimulation of immune responses. For example, the production of T_{H}1 cytokines is thought to promote cellular immunity with antitumor, antiviral/antibacterial, and adjuvant activities, whereas T_{H}2 cytokine production is thought to subdue autoimmune diseases and promote antibody production.

Many of these cells recognize the non-polymorphic CD1d molecule, an antigen-presenting molecule that binds self- and foreign lipids and glycolipids. The TCR of the NK-T cells are able to recognize glycolipid antigens presented (chaperoned) by a CD1 d molecule. A major response of NK-T cells is rapid secretion of cytokines, including IL-4, IFN-γ and IL-10 after stimulation and thus influence diverse immune responses and pathogenic processes. The NK-T cells may be a homogenous population or a heterogeneous population. In one exemplary implementation, the population may be “non-invariant NK-T cells”, which may comprise human and mouse bone marrow and human liver T cell populations that are, for example, CD1d-reactive noninvariant T cells which express diverse TCRs, and which can also produce a large amount of IL-4 and IFN-γ. The best known subset of CD1d-dependent NK-T cells expresses an invariant TCR-alpha (TCR-t) chain. These are referred to as type I or invariant NK-T cells (iNK-T cells). These cells are conserved between humans (Vα24i NK-T cells) and mice (Vα14i NK-T cells) and are implicated in many immunological processes.

### γδ-T Cells

The major populations, including CD4⁺and CD8⁺subsets, express a receptor composed of alpha and beta chains. A small subset express a TCR made from gamma and delta chains. In some embodiments, the T cell is a gamma delta (γδ) T cells. Gamma delta (γδ) T cells make up 3-10% circulating lymphocytes, and the Vδ2+ subset makes up 75% of γδ-T cells in blood. Vδ2+ cells recognize non-peptide epitopes and do not require antigen presentation by MHCs or HLA. The majority of Vδ2+ T cells also express a Vγ9 chain and are stimulated by exposure to 5-carbon pyrophosphate compounds that are intermediates in mevalonate and non-mevalonate sterol/isoprenoid synthesis pathways. The response to isopentenyl pyrophosphate (5-carbon) is universal among healthy human beings. Another subset of γδ-T cells, Vδ+, make up a much smaller percentage of the T cells circulating in the blood, but Vδ+1 cells are commonly found in the epithelial mucosa and the skin.

In general, γδ-T cells have several functions, including killing tumor cells and pathogen-infected cells. Stimulation through their unique TCRs composed of two glycoprotein chains, γ and δ, improves the capacity for cellular cytotoxicity, cytokine secretion and other effector functions. The TCRs of γδ-T cells have unique specificities, and the cells themselves occur in high clonal frequencies, thus allowing rapid innate-like responses to tumors and pathogens.

### NK Cells

In some embodiments, the immune cell is a natural-killer (NK) cell. NK cells are a sub-population of lymphocytes, involved in non-conventional immunity. Characteristics and biological properties of NK cells include the expression of surface antigens including CD16, CD56, and/or CD57; the absence of the alpha/beta or gamma/delta TCR complex on the cell surface; the ability to bind to and kill cells that fail to express “self” MHC/HLA antigens by the activation of specific cytolytic enzymes; the ability to kill tumor cells or other diseased cells that express a NK activating receptor-ligand; the ability to release cytokines that stimulate or inhibit the immune response; and the ability to undergo multiple rounds of cell division and produce daughter cells with similar biologic properties as the parent cell. Within the context of this invention “active” NK cells designate biologically active NK cells, more particularly NK cells having the capacity of lysing target cells. For instance, an “active” NK cell is able to kill cells that express an NK activating receptor-ligand and fail to express “self ” MHC/HLA antigens (KIR-incompatible cells).

NK cells are negatively regulated by major histocompatibility complex (MHC) class I-specific inhibitory receptors (Kärre et al., 1986; Öhlén et al, 1989). These specific receptors bind to polymorphic determinants of MHC class I molecules or HLA present on other cells and inhibit NK cell lysis.

IIIb. Isolation of Immune Cells

Immune cells can be isolated using any appropriate method known in the art. For example, T cells can be isolated from human peripheral blood mononuclear cells (PBMCs), PBMCs collected after stimulation with G-CSF, bone marrow, or umbilical cord blood. NK cells can be isolated from human PBMCs or umbilical cord blood. Immune cells can also be differentiated from stem or progenitor cells, such as embryonic stem cells (ESCs), induced pluripotent stem cells (iPSCs), or hematopoetic progenitor cells (HPCs) for example. In some embodiments, immune cells are primary cells isolated from a patient prior to manipulation. In some embodiments, immune cells are derived from established cell lines that have been propagated ex vivo. Non-limiting examples of cell lines used in the present invention include NK-70 NK cells and Jurkat T cells.

In some embodiments, isolated immune cells are autologous. “Autologous” cells are administered to the same subject from whom they were isolated. In some embodiments, isolated immune cells are allogenic. “Allogenic” (or “heterologous”) cells are administered to a different subject than the subject from whom they were isolated. Isolated immune cells can be propagated ex vivo for a period of time ranging from one or more days to weeks or longer.

IIIc. Expression of Synthetic Receptors within Immune Cells

The synthetic receptors disclosed herein can be produced using methods known to those skilled in the art. In some embodiments, isolated immune cells comprise a nucleic acid encoding the sequence of a synthetic receptor described herein and are known as “recombinant” cells. In some embodiments, the nucleic acid is an RNA molecule that is translated into a synthetic receptor protein. In some embodiments, the nucleic acid is a DNA molecule that encodes the synthetic receptor protein (i.e., the DNA is transcribed into mRNA, which is translated into a synthetic receptor protein. In some embodiments, a nucleic acid sequence will further comprise a “signal sequence” that causes the synthetic receptor to be localized to the surface of a cell. In some embodiments, the signal sequence can be the endogenous signal sequence of N-terminal component of the synthetic receptor. In some embodiments, the signal sequence can be a different signal sequence that that of the synthetic receptor. However, the signal sequence selected should be compatible with the secretory pathway of the immune cell so that the receptor is presented on the cell surface.

In some embodiments, a vector comprising the nucleic acid sequence encoding the synthetic receptor is used to deliver the nucleic acid into immune cells. Nucleic acid vectors can be designed to deliver desired genes to immune cells under the control of regulated eukaryotic promoters, for example, MNDU3 promoter, CMV promoter, EF1alpha promoter, or Ubiquitin promoter. Also, the vectors may contain a selectable marker, if for no other reason, to facilitate their manipulation in vitro.

Nucleic acids or nucleic acid vectors can be delivered into isolated immune cells using any suitable method known to those of ordinary skill in the art. In one non-limiting example, nucleic acid vectors comprising a nucleic acid sequence encoding a synthetic receptor can be designed to produce a virus. Viruses can be produced using packaging cell lines, then contacted to isolated immune cells, causing the isolated immune cells to express the synthetic receptor proteins. Non-limiting examples of suitable viruses include retroviruses, lentiviruses, and adenoviruses. In another non-limiting example, a nucleic acid or nucleic acid vector can be delivered into an isolated immune cell using a transfection reagent. The transfection reagent allows the nucleic acid to penetrate the cell membrane, where it will be transcribed and/or translated to produce a synthetic receptor. Non-limiting examples of transfection reagents include cationic polymers (e.g., polyethylenimine), cationic lipids (e.g., lipofectamine 2000), and other reagents such as FuGENE (Fugent, LLC., Madison, WI, USA) or calcium phosphate.

Immune cells used in the present invention can be expanded before administration to a subject using any appropriate method known to those of ordinary skill in the art. In some embodiments, immune cells are expanded prior to expression of the synthetic receptor. In some embodiments, immune cells are expanded after expression of the synthetic receptor. In some embodiments, expansion of recombinant immune cells occurs subsequent to their activation.

IIId. Activation of Recombinant Immune Cells

Recombinant immune cells expressing synthetic receptors described herein can be activated using any suitable method known to those of ordinary skill in the art. For example, recombinant immune cells can be contacted to antigen-presenting cells (APCs). In some embodiments, recombinant immune cells can activated ex vivo, for example, by contacting them with APCs presenting the antigen recognized by the expressed synthetic receptor. In some embodiments, naive recombinant immune cells can be administered to a subject to be activated in vivo.

Professional antigen-presenting cells, which include macrophages, B lymphocytes, and dendritic cells, are distinguished by their expression of MHC class-II proteins. By contrast, all nucleated vertebrate cells express MHC class-I molecules and can also serve as APCs. APCs internalize antigen and re-express a part of that antigen, together with the MHC molecule on their outer cell membrane. T helper lymphocytes generally recognize antigen associated with MHC class II molecules, and T cytotoxic lymphocytes recognize antigen associated with MHC class I molecules. In humans, the MHC is referred to as the HLA complex and in mice the H-2 complex.

### IV. Treatment with Recombinant Immune Cells

IVa. Subjects and Diseases

As detailed herein, CAR-immune cells of the embodiments can be used to treat a wide range of diseases and conditions in a subject. Essentially any disease that involves the specific or enhanced expression of a particular antigen can be treated by targeting CAR cells to the antigen. For example, infectious diseases and cancers can be treated with methods and/or compositions of the invention. These include cancers, such as primary, metastatic, recurrent, sensitive-to-therapy, refractory-to-therapy cancers (e.g., chemo-refractory cancer). The cancer may be of the blood, lung, brain, colon, prostate, breast, liver, kidney, stomach, cervix, ovary, testes, pituitary gland, esophagus, spleen, skin, bone, and so forth (e.g., B-cell lymphomas or a melanomas). In the case of cancer treatment CAR cells typically target a cancer cell antigen (also known as a tumor-associated antigen (TAA)).

IVb. Tumor Antigens

In some embodiments, the targeting domain of the synthetic receptor (which may be referred to as an antigen-binding domain) is designed to target a tumor-associated antigen. A TAA may be of any kind so long as it is expressed on the cell surface of tumor cells. Non-limiting exemplary embodiments of tumor associated antigens include CD19, mesothelin, CD123, BCMA, GD2, CD30, GPC3, CD22, HER2, HER3, CD20, EGFR, carcinoembryonic antigen, alpha-fetoprotein, CA-125, MUC-1, CD56, c-Met, AKT, epithelial tumor antigen, melanoma-associated antigen, Flt3, CD33, Muc-16, CS1, and a tumor-associated neoantigen. A “neoantigen” is a mutated protein that is present in some or all cells of a tumor, but not present in non-tumor cells.

In some embodiments intracellular tumor associated antigens may be targeted, such as HA-1, survivin, WT1, and p53. This can be achieved, for example, by a synthetic receptor expressed on a universal T cell that recognizes the processed peptide described from the intracellular tumor associated antigen in the context of HLA. In addition, the universal T cell can be genetically modified to express a T-cell receptor pairing that recognizes the intracellular processed tumor associated antigen in the context of HLA.

In some embodiments, the synthetic receptor can be co-expressed with a membrane-bound cytokine to improve persistence when there is a low amount of tumor-associated antigen. For example, a CAR can be co-expressed with membrane-bound IL-15.

In some embodiments the cancer is a solid tumor. In some embodiments, the cancer is a hematopoetic malignancy. In some embodiments, the cancer is selected from, without limitation, epithelial neoplasia, squamous cell neoplasia, squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, Hurthle cell adenoma, renal cell carcinoma, Grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, Warthin's tumor, thymoma, specialized gonadal neoplasms, sex cord stromal tumor, thecoma, granulosa cell tumor, arrhenoblastoma, Sertoli-Leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, malignant acral lentiginous melanoma, Askin's tumor, botryodies, chondrosarcoma, malignant hemangioendothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcomagerminoma, dysgerminoma, seminoma, nongerminomatous germ cell tumor, embryonal carcinoma, endodermal sinus tumor, choriocarcinoma, teratoma, polyembryoma, gonadoblastoma, nephroblastoma, medulloblastoma, retinoblastoma, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. Hematopoetic malignancies include, without limitation, chronic lymphocytic leukemia/small lymphocytic lymphoma, B-cell prolymphocytic leukemia, lymphoplasmacytic lymphoma (such as Waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical Hodgkin's lymphomas (nodular sclerosis, mixed cellularity, lymphocyte-rich, lymphocyte depleted or not depleted), and nodular lymphocyte-predominant Hodgkin's lymphoma.

IVc. Infectious Disease Antigens

In some embodiments, the targeting domain of the synthetic receptor is designed to target an antigen from an infectious disease-causing organism (i.e., an infectious agent). In some embodiments the infectious agent is a bacterium, a virus, a fungus, or a parasite. In some embodiments, the infections disease is a bacterial infection, a viral infection, a fungal infection, or a parasitic infection. In some embodiments, the infectious agent is a bacterium. Examples of infection-causing bacteria include, but are not limited to Bacillus anthracis, Bordetella pertussis, Borrelia burgdorferi, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani, Corynebacterium diphtheria, Enterobacter sakazakii, Enterococcus faecalis, Enterococcus faecium, Escherichia coli, Francisella tularensis, Haemophilus influenza, Helicobacter pylori, Legionella pneumophila, Leptospira interrogans, Listeria monocytogenes, Mycobacterium leprae, Mycobacterium tuberculosis, Mycobacterium ulcerans, Mycoplasma pneumonia, Neisseria gonorrhoeae, Neisseria meningitides, Pseudomonas aeruginosa, Rickettsia ricketisia, Salmonella typhi and Salmonella typhimurium, Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Vibrio cholera, Yersinia enterocolitica, and Yersinia pestis.

In some embodiments, the infections agent is a virus. Examples of infection-causing viruses include, but are not limited to the families Adenoviridae, Picornaviridae, Herpesviridae, Hepadnaviridae, Flaviviridae, Retroviridae, Coronaviridae, Papilliomaviridae, Reoveridae, Hepeveridae, Orthomyxoviridae, Paramyxoviridae, Papovaviridae, Polyomaviridae, Pneumoviridae, Filoviridae, Parvoviridae, Poxviridae, Rhabdoviridae, and Togaviridae.

In some embodiments, the infectious agent is a fungus. Examples of infection-causing fungi include, but are not limited to the genera Aspergillus, Candida, Cryptococcus, Fusarium Histoplasma, Basidiobolus, Conidiobolus. Pneumocystis, Mucor, Rhizopus, Absidia, Cunninghamella, Trichophyton, Microsporum, Epidermophyton and Stachybotrys.

In some embodiments, the infectious agent is a parasite (e.g., a protozoan, an amoeba). Examples of infection-causing parasites include, but are not limited Acanthamoeba, Balamuthia mandrillaris, Cryptosporidium canis, Cryptosporidium fells, Cryptosporidium hominis, Cryptosporidium meleagridis, Cryptosporidium muris, Cryptosporidium parvum, Dientamoeba fragilis, Endolimax nana, Entamoeba dispar, Entamoeba hartmanni, Entamoeba histolytica, Entamoeba coli, Entamoeba moshkovskii, Giardia lamblia, Iodamoeba butschlii, Leishmania aethiopica, Leishmania braziliensis, Leishmania chagasi, Leishmania donovani, Leishmania infantum, Leishmania major, Leishmania mexicana, Leishmania tropica, Naegleria fowleri, Plasmodium falciparum, Plasmodium knowlesi, Plasmodium malariae, Plasmodium ovale, Plasmodium vivax, Sappinia diploidea, Toxoplasma gondii, Trichomonas vaginalis, Trypanosoma brucei, Trypanosoma cruzi Taenia solium, Taenia saginata, Hymenolepis., Echinococcus granulosus, Echinococcus multilocularis, Multiceps multiceps, Schistosoma mansoni, Schistosoma japonicum, Fasciola hepatica, Ascaris, Enterobius, Filarioidea, Onchocerca, Rhabditis, Trichuris, Necator americanus, and Ancylostoma.

IVd. Pharmaceutical Compositions

Provided herein, in various embodiments are pharmaceutical compositions including the recombinant immune cells expressing the synthetic receptor. The pharmaceutical compositions generally include one or more optional pharmaceutically acceptable carrier or excipient. In some embodiments, the composition includes at least one additional therapeutic agent.

In various embodiments, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (e.g., octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents, in some embodiments, are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known to those skilled in the art. Exemplary methods are described in more detail in, for example, Remington: The Science and Practice of Pharmacy, Lippincott Williams & Wilkins; 21st ed. (May 1, 2005).

The formulation or composition may also contain more than one active ingredients useful for the particular indication, disease, or condition being treated with the active recombinant cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents (e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc), antibiotic agents (e.g., penicillins, cephalosporins, polymyxins, rifamycins, lipiarmycins, quinolones, sulfonamides, macrolides, lincosamides, tetracyclines, etc.), antiviral agents (e.g., abacavir, adefovir, baloxavir marboxil, cobicistat, doravirine, efavirenz, etravirine, imiquimod, methisazone, nitazoxanide, oseltamivir, peramivir, remdesivir, umifenovir, valaciclovir, valganciclovir, zanamivir, etc.), antifungal agents (e.g., polyenes, azoles, allylamines, echinocandins, etc.), or antiparsitic drugs (e.g., nitazoxanide, malarsoprol, eflornithine, metronidazole, tinidazole, miltefosine, mebendazole, pyrantel pamoate, albendazole, praziquantel, rifampin, etc.). In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition, in some embodiments, contains the active recombinant immune cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, and the like, depending on the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

IVe. Dosing and Administration

Also provided herein, in various embodiments, are methods for using and uses of the active recombinant cells expressing the synthetic receptor. Such methods and uses include therapeutic methods and uses, for example, involving administration of cells or compositions containing the cells, to a subject having a disease, condition, or disorder. In some embodiments, the cell and/or composition is administered in an effective amount to effect treatment of the disease or disorder. Uses include uses of the cells in such methods and treatments, and in the preparation of a medicament in order to carry out such therapeutic methods. In some embodiments, the methods are carried out by administering the cells or compositions comprising the cells to the subject having or suspected of having the disease or condition. In some embodiments, the methods thereby treat the disease or condition or disorder in the subject.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10):928-933; Tsukahara et al. (2013) Biochem Biophys Res Commun 438(1):84-9; Davila et al. (2013) PLoS ONE 8(4):e61338.

The recombinant cells may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo, or in vitro-derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

In some embodiments, in the context of recombinant cells expressing the synthetic receptor, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject.

In some embodiments, the recombinant cells are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

### EXAMPLES

### Example 1—Activation of T Cells with a KIR2DL4-Based Synthetic Receptor

A chimeric antigen receptor (CAR) comprising the transmembrane (TM) and intracellular signaling domains (ICD) of KIR2DL was transduced into 4 Jurkat T cells with lentiviral vectors, along with a negative control CAR without an antigen-binding domain, positive control CAR with CD3z signaling domain. Transduced cells were then cultured for 2 days before a portion of cells was harvested and stimulated by co-culturing with naturally CD19-expressing Raji cells in a 3:1 ratio (Raji:Jurkat). Both non-stimulated and stimulated cells were then cultured for additional 24 hours prior to flow cytometry analysis. The expression of the integrated lentiviral transgene encoding receptors was measured as fluorescence of mCherry protein expressed from the same vector. The immune activation was measured as fluorescence of NFAT-NFkB gene reporter that leads to expression of mCitrine-PEST short-lived fluorescent protein upon immune stimulation.

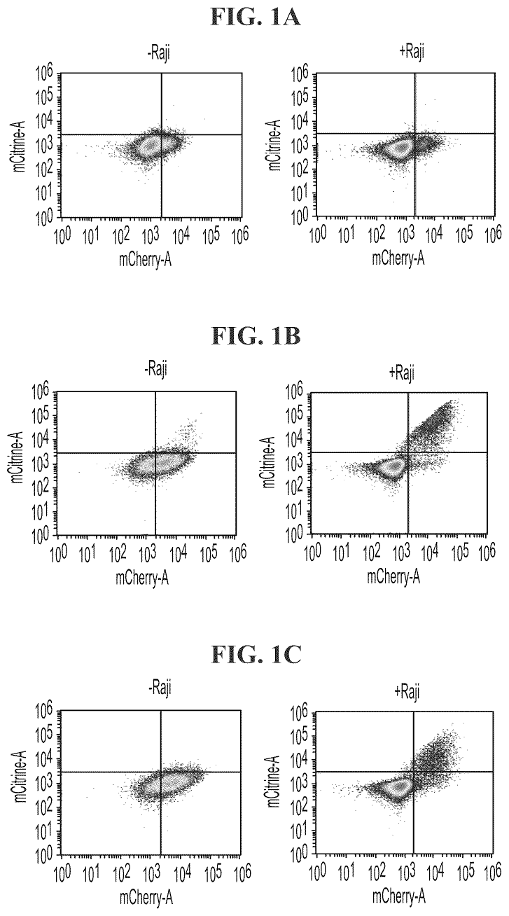

As shown in FIG. 1A, cells expressing a CAR lacking an antigen-binding domain were not activated by exposure to CD19-expressing cells. Cells expressing the positive-control, CD3z-containing CAR were strongly activated following exposure to the Raji cells (FIG. 1B). Finally, cells expressing a KIR2DL-based CAR were also strongly activated following exposure to Raji cells. These results indicate that KIR2DL4-based CARs can promote antigen-specific activation of T cells.

### Example 2—Characterization of the KIR2DL4 Intracellular Signaling Domain

To determine the portion of the KIR2DL4 ICD that is necessary and/or sufficient for immune cell activation, 9 C-terminal, 7 N-terminal, and 6 N-terminal and C-terminal KIR2DL4 ICD truncation mutants were generated based on predicted sequence features by progressively removing 6-20 amino acids. The ITIM motif in KIR2DL4 (VTYAQL) is present at positions 36-41 of the full-length ICD (SEQ ID NO:2). Further mutants comprising synthetic receptors comprising the truncation mutants were then transduced into Jurkat T cells via lentiviral vectors. These transduced cells were then cultured for 2 days before a portion of cells was harvested and mixed with naturally CD19-expressing Raji cells in 3:1 ratio (Raji:Jurkat). Both non-stimulated and stimulated cells were cultured for additional 24 hours before flow cytometry analysis.

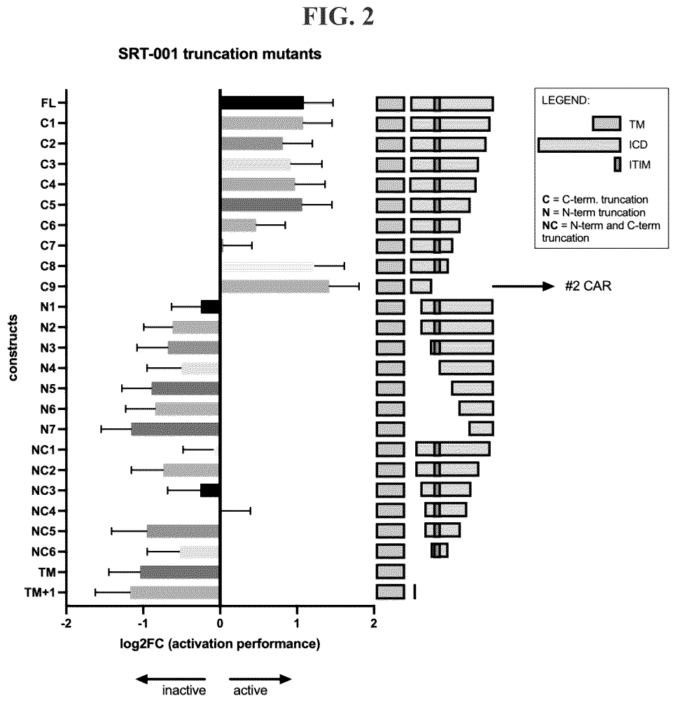

As shown in FIG. 2, a synthetic receptor comprising C-terminal truncation mutant that contained the first 29AA of the ICD cells (C9: “truncation A” or “#2 CAR”) had strong ability to activate T cells. Furthermore a CAR comprising a KIR2DL4 ICD lacking the first 10 amino acids led to a reduced ability to induce T cell activation (N1). These data show that combination of a portion of the N-terminal region of the ICD 10-29 amino acids in length is essential for the immune activation function of the synthetic receptor.

To further characterize the essential portions of the KIR2DL4-based synthetic receptors. Multiple variants of synthetic receptors, including a negative control CAR lacking the ICD (no ICD CAR), a positive control CAR with a CD3z ICD, and CARs comprising full-length KIR2DL4 TM-ICD (#1 CAR), KIR2DL4 TM with KIR2DL4 ICD truncation A (#2 CAR), KIR2DL4 TM with KIR2DL4 ICD ITIM substitution mutant (#3 CAR), KIR2DL4 TM with no ICD (#4 CAR), and CD8a TM with KIR2DL4 ICD (#5 CAR). These synthetic receptors were then transduced into Jurkat T cells via lentiviral vectors. Transduced cells were then cultured for 2 days before being stimulated by co-culturing with naturally CD19-expressing Raji cells in a 3:1 ratio (Raji:Jurkat). The stimulated cells were further cultured for additional 24 hours before FACS analysis. 100,000 of each of the highest (positive) and the lowest activated cells were isolated and the constructs contained in these cells were identified using next-generation sequencing. The ratio of the constructs in the positive and negative population of cells is expressed as log2 fold-change. The experiment was performed in three independent replicates.

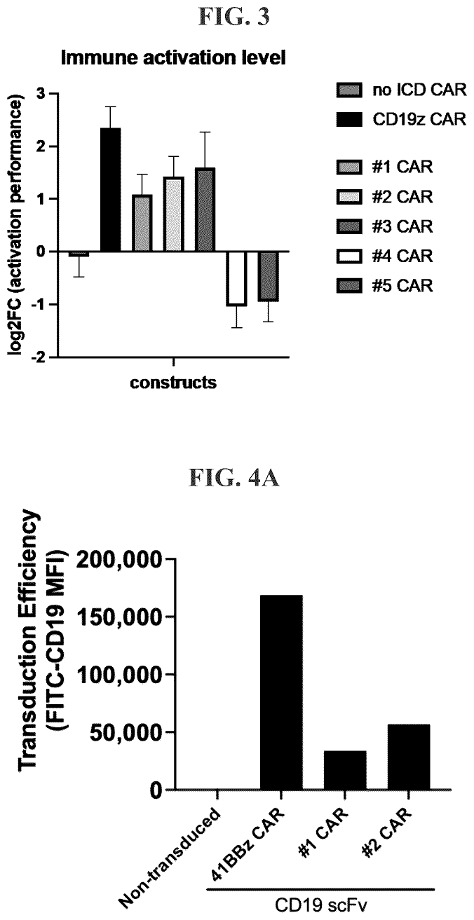

As shown in FIG. 3, synthetic receptors comprising the KIR2DL4 TM domain and either the full length KIR2DL4 ICD, the first 29 amino acids, or the full-length ICD with mutations in the ITIM have strong abilities to activate T cells. Completely removing the ICD and leaving only the KIR2DL4 TM domain (#4 CAR) is not sufficient to induce any activity. Interestingly, removing the KIR2DL4 TM and replacing it with CD8a TM (#5) completely abolishes activity even in presence of full-length KIR2DL4 ICD.

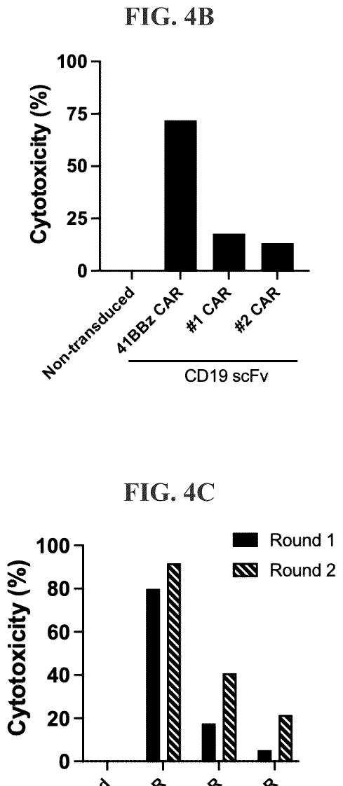

In another experiment, primary T cells were mock treated (Non-transduced) or transduced with the indicated chimeric receptors. These constructs were cloned with the CD19 scFv. At the end of manufacturing, transduction efficiencies are measured with a FITC-conjugated CD19 protein and reported as MFI. Transduced T cells are then exposed at a 1:1 effector-to-target ratio to luciferase-expressing Raji cells to measure cytotoxicity (FIG. 4A). Short-term cytotoxicity is measured after 24 hours (FIG. 4B). Repetitive cytotoxicity is measured by performing 2 consecutive rounds of Raji cell exposure every 48 hours (FIG. 4C). In both cases cytotoxicity was calculated by normalizing the killing in each condition to wells containing target cells alone.

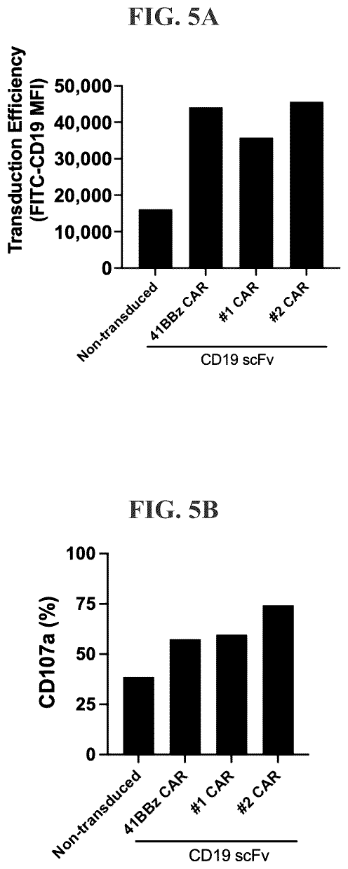

Primary NK cells were mock treated (Non-transduced) or transduced with the indicated chimeric receptors. These constructs were cloned with the CD19 scFv. At the end of manufacturing, transduction efficiencies are measured with a FITC-conjugated CD19 protein and reported as MFI. Transduced NK cells are then exposed to Raji cells at a 1:1 effector-to-target ratio for 1 hour to induce functional degranulation (FIG. 5A). Degranulation is measured by Flow Cytometry-based detection of CD107a on the cell surface (FIG. 5B).

Taken together, these results show that the KIR2DL4 transmembrane domain as well as the first 10-29 amino acids are necessary for activation of immune cells by KIR2DL4-based synthetic receptors. These results also demonstrate that synthetic receptors comprising this domain can support short-term and sustained immune cell activation.

### Example 3—Combination of KIR2DL4-Based Synthetic Receptor Activation with Costimulatory Domains

Engineered Jurkat T cells were mock treated (Non-transduced) or transduced with lentiviral vectors encoding two positive control CARs with either CD28 (28z) or 4-1BB (41BBz) co-stimulation domains; or with different chimeric receptors containing KIR2DL4 domains. Receptors included: KIR2DL4 TM with a C8-truncated [KIR2DL4(C8)] ICD domain (#6 CAR, SEQ ID NO:32); WT KIR2DL4 TM with KIR2DL4(C8) and 4-1BB ICD domains (#7 CAR; SEQ ID NO: 33); and WT KIR2DL4 TM with KIR2DL4(C8) and DAP10 ICD domains (#8 CAR; SEQ ID NO: 44). These constructs were cloned with CD19 or MSLN scFvs.

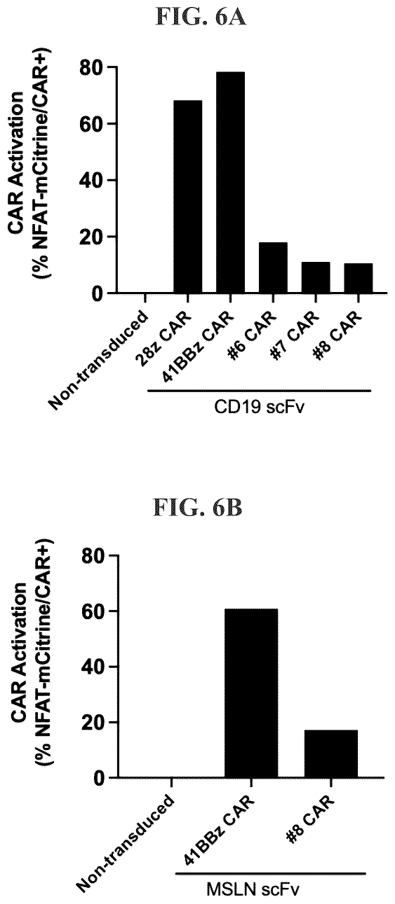

In one experiment, transduced cells were cultured for 2 days before a portion of cells was harvested and mixed with naturally CD19-expressing Raji cells or MSLN-expressing K562 cells at a 1:1 effector-to-target cell ratio for 24 hours. Immune activation was measured by Flow Cytometry as the fluorescence of NFAT-NFkB gene reporter that leads to expression of mCitrine-PEST short-lived fluorescent protein upon immune stimulation. Data is expressed as percent of cells expressing mCitrine as measured by Flow Cytometry. FIG. 6A shows activation of CD19-CAR immune cells against CD19⁺ target cells. FIG. 6B shows activation of MSLN-CAR immune cells against MSLN target cells.

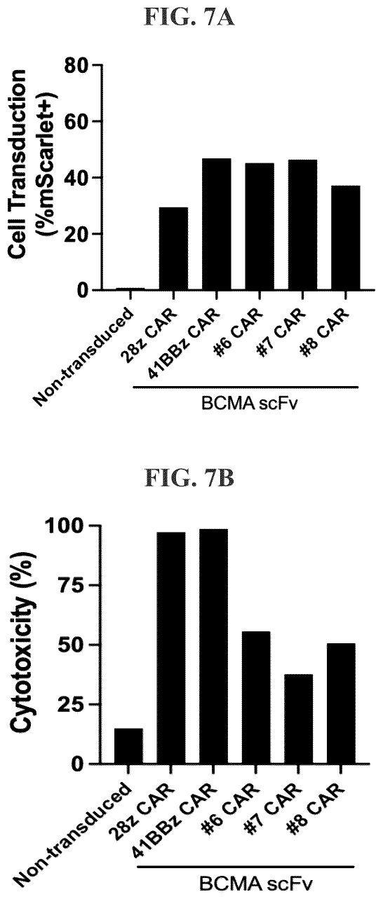

In another experiment, primary T cells were mock treated (non-transduced) or transduced with the indicated chimeric receptors. These constructs were cloned with the BCMA scFv. At the end of manufacturing, transduction efficiencies are measured by the expression of the mScarlet fluorescent reporter protein (FIG. 7A). Transduced T cells are then exposed to BCMA-expressing K562 cells at a 5:1 effector-to-target ratio for 24 hours to measure cytotoxicity by Flow Cytometry. Cytotoxicity was calculated by normalizing the killing in each condition to wells containing target cells alone (FIG. 7B).

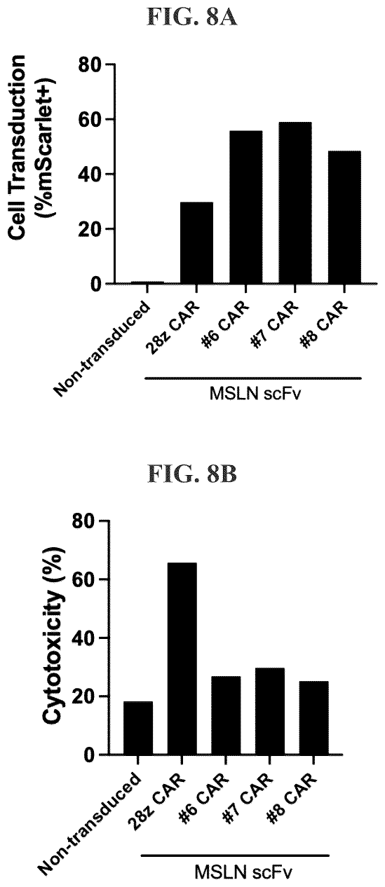

In another experiment, primary T cells were mock treated (non-transduced) or transduced with the indicated chimeric receptors. These constructs were cloned with the MSLN scFv. At the end of manufacturing, transduction efficiencies are measured by the expression of the mScarlet fluorescent reporter protein (FIG. 8A). Transduced T cells are then exposed to MSLN-expressing K562 cells at a 5:1 effector-to-target ratio for 24 hours to measure cytotoxicity by Flow Cytometry. Cytotoxicity was calculated by normalizing the killing in each condition to wells containing target cells alone (FIG. 8B).

These results indicate that costimulatory domains such as CD3z, 4-1BB, or DAP10 are compatible with KIR2DL4-based synthetic receptors and can be used to support immune cell activation.

### Incorporation by Reference

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of activating a recombinant immune cell expressing a synthetic receptor, comprising:
(a) an antigen-binding domain;
(b) a transmembrane domain; and
(c) an intracellular signaling domain comprising a sequence of 10-29 amino acids derived from killer cell immunoglobulin-like receptor 2DL4 (KIR2DL4);
wherein the method comprises binding an antigen to the antigen-binding domain, thereby activating the recombinant immune cell.

2-68. (canceled)

69. A recombinant immune cell, comprising: an expressed synthetic receptor, the receptor comprising:
(a) an antigen-binding domain;
(b) a transmembrane domain; and
(c) an intracellular signaling domain comprising a sequence of 10-29 amino acids derived from killer cell immunoglobulin-like receptor 2DL4 (KIR2DL4);
wherein the recombinant immune cell is activated by binding a cognate antigen to the antigen-binding domain.

70. The recombinant immune cell of claim 69, wherein the intracellular signaling domain comprises an amino acid sequence of SEQ ID NO: 8 or 9.

71. (canceled)

72. The recombinant immune cell of claim 69, wherein the intracellular signaling domain further comprises an amino acid sequence of any one of SEQ ID NOs: 12-16.

73-77. (canceled)

78. The recombinant immune cell of claim 69, wherein the transmembrane domain comprises an amino acid sequence of SEQ ID NO: 1, 2, or 3.

79-81. (canceled)

82. The recombinant immune cell of claim 69, wherein the synthetic receptor further comprises a hinge region interposed between the antigen-binding domain and the transmembrane domain.

83. The recombinant immune cell of claim 82, wherein the hinge region comprises an amino acid sequence of SEQ ID NO: 4 or 5.

84-85. (canceled)

86. The recombinant immune cell of claim 69, wherein the synthetic receptor comprises an amino acid sequence having at least 90% sequence identity with an amino acid sequence of any one of SEQ ID NOs: 17-34.

87-104. (canceled)

105. The recombinant immune cell of claim 69, further comprising the cognate antigen bound to the antigen-binding domain, wherein the cell is in an active state.

106. The recombinant immune cell of claim 69, wherein the antigen-binding domain comprises an antibody or functional fragment thereof.

107. The recombinant immune cell of claim 69, wherein the antigen-binding domain comprises a single-chain variable fragment (scFv), a minimal active antibody fragment, a single domain antibody, a single light chain variable domain, a single heavy chain variable domain, or a nanobody.

108-112. (canceled)

113. The recombinant immune cell of claim 69, wherein the recombinant immune cell is a T cell, an NK cell, or an NK-T cell.

114-116. (canceled)

117. A method of treating a disease in a subject in need thereof, the method comprising administering to the subject the recombinant immune cell of claim 69.

118. (canceled)

119. The method of claim 117, wherein the subject is a human.

120-121. (canceled)

122. The method of claim 117, wherein the disease is a cancer.

123. The method of claim 122, wherein the cancer is a hematopoietic malignancy or a solid tumor.

124. (canceled)

125. The method of claim 122, wherein the antigen is a whole protein or a fragment thereof of CD19, mesothelin, CD123, BCMA, GD2, CD30, GPC3, CD22, HER2, CD20, EGFR, Flt3, CD33, Muc-16, CS1, or a tumor neoantigen.

126-141. (canceled)

142. The method of claim 117, wherein the disease is an infectious disease caused by an infectious agent.

143. The method of claim 142, wherein the infectious agent is a bacterium, a virus, a fungus, or a parasite.

144. (canceled)

145. A recombinant immune cell, comprising: an expressed synthetic receptor, the receptor comprising:
(a) means for binding an antigen of interest;
(b) a transmembrane domain; and
(c) an intracellular signaling domain comprising a sequence of 10-29 amino acids derived from killer cell immunoglobulin-like receptor 2DL4 (KIR2DL4);
wherein the recombinant immune cell is activated by binding the antigen of interest to the synthetic receptor.
